SEQUENCE LISTING (38 sequences)

SEQ ID NO: 1 - DNA, 889 bp, Homo sapiens
ctcttctcattccacaagctctccccaaaagaccccattcctccccaccttcaaccatctctggcagggaggagggggaactgagaggctactttctgacccaggaccctaagcctgtgtacggagagagcatgagctgggtgagctgcttgccaaggagtggcatctgccctcatcagtggacacaaaaagccccaggggttaagtggccatggctgccctcatggctgcaccgggaggatgactgtgttcccactctcagtcctgccgaggtgcctgacagccctgcacccaggagctggggggtctaagagcttgtaaaaagtgtacaagtgccagatgctcgttgtgcacaaatctaaatgcagaaaagcactgaaagaagaatccagaaaaccacagttcccatttttatatgggagcaaacaaaggcagatcccaagctcttcctcttccctagatcaatacagacagacagacaggtggatagatagatagatagatagatagatagatagatagatagatatcattgaaagacaaaacagagatggatgatagatacatgcttacagatgcacacacaaacgctaaatggtataaaaatggaatcactctgtaggctgttttaccacctactttactaaattaatgagttattgagtataatttaattttatatactaatttgaaactgtgtcattaggtttttaagtctatggcatcactttcgcttgtatttttctattgatttcttttcttttcttttcttttttgagacagagtctcactctcacccaggctggagtaccgtggcacgatcttggctcattgcaaccaccacctcccgggctcaagtgattatcctgcctcagcctcccaaatagct

SEQ ID NO: 2 - DNA, 843 bp, Homo sapiens
atatgctaactggatgtgaacaattgtgttctaatgagcttaatatgagtttcataatttgtgcattttgctgttaaaaagccagaaaacaaaacaaaacaaaatactgaaaccagtgtgaacaagagttacacgatggaaggcatcagttttcacaccagaaggaataaaaacaggcaaaaataccataagttgatcctcaaaatatgattgattttaagccttatgagataattgtgaggtcttaaaatctgaggtatcaaaaactcagagggaatatatattcttaagaattataacgattccacatttatcctcattgacagaattgcaccaaatattggtaattaaatgtttactatagactatttagtgagattaaaaaaaactatcaatctgtctatctatctatctatctatctatctatctatctatctatctatctatctatcgttagttcgttctaaactatgacaagtgttctatcataccctttatatatattaaccttaaaataactccatagtcagcctgaccaacatggtgaaaccccgtctctaaaaaaaatacaaaaattagctggatgcagtagcacatgcctgtagtcccagctactcaggaggctggggcaggagaaccacttgacccaagaagcggaggttgcagtgagccgagatcgcaccactgcactccagcctgggtgacagagtgagactccatctcaagataaagaaataaataaaaacaaacaaacaaaaaaattccatagggggtcaggtgcggtggctcatgcctgtaatcccagcactttgggaggccgaagcaggtggatcacttgaggt

SEQ ID NO: 3 - DNA, 792 bp, Homo sapiens
aatatgaattcaatgtatacagagagagagagagagagagagagagagagagacttctacagagctctaagcataattgtgtaactccaagctcacagtgcctaagaccagtaccaggctgactcattggaaagctgccatagtaagactcttctgttcactgcattatttattgatgtattgcaagcacttagttacatttctagcatataacacatgatcaataaatattttgacatgaacaaatggtaattctgcctacagccaatgtgaatattgggatgggttgctggacatggtatcacagaagtctgggatgtggaggagagttcatttctttagtgggcatccgtgactctctggactctgacccatctaacgcctatctgtatttacaaatacattatctatctatctatctatctatctatctatctatctatctatcaatcaatcatctatctatctttctgtctgtctttttgggctgcctatggctcaacccaagttgaaggaggagatttgaccaacaattcaagctctctgaatatgttttgaaaataatgtatattaatgaatgtacaaatttccccacttgtactttcagactgttatctgtgagttaaaactcctccactctttttcctacccaaataatagcatacttttttctgagtatattttgggaagaagagttattcagttattgttatattttaaaaaattccttataccaaactctacttgatctaaggctattcattgaaactttcagcatgcttaatagcagtc

SEQ ID NO: 4 - DNA, 735 bp, Homo sapiens
ccctctgtgtagcctggctatgtgccacattgtgaggttctctccctctagctacttcttccagtttcctctttcaggatcccaattcctttctcaaagcactggtgaataactccaaatactccatcatttcattatacagagtaatataagtcttctttttctaaacctctcccatctggatagtggacctcatatttcagatgctaataggctgttgaggtagtttcctaagcaaaaaagtaattgtctctctcagaggaatgctttagtgctttttagccaagtgattccaatcatagccacagtttacaacatttgtatctttatctgtatccttatttatacctctatctatctatctatctatctatctatctatctatctatctatcttcaaaatattacataaggataccaaagaggaaaatcacccttgtcacatacttgctattaaaatatacttttattagtacagattatctgggacaccactttaattagaagctttaaaagcatatgcatgtctcagtatttaattttaaaattattacataattatatactcctttgaattagaaaattacaaatgtggctatgtattattttctcccatgtattttcaaaatgagggggtaagccagaccctctccctctcccatgcctaatagctcaaagttaaaggtaaagaaacaagaaaatatggtgaaagttaaccagcttcacttcagagga

SEQ ID NO: 5 - DNA, 949 bp, Homo sapiens
attcaacacatgaggcacggccagacctaaatgtctcagagcctgctcccactccgatgagctgctgccttgcttcagggtctgagtccagtgactgccactgcctgcacccaatcaccatagccagagacctggaggtcatccttatctcctttctcttcctcatccctgcatctcagactcttccacacaccactggccatcttcagcccattctccagcctccaggttcccacccaacccacatggtgccagactgagccttctcagatactatctcctggtgcacacttggacagcatttcctgtgtcagaccctgttctaagtacttcctatctatctatctatctatctatctatctatctatctatctatctatctatctaatctatctatcttctatctatgaaggcagttactgttaatatcttcattttacaggtaggaaaactgagacacagggtggttagcaacctgctagtccttggcagactcaggttggaacactgccctggagtgtgtgctcctgaccaccacgaagtgcctcctctgtacaatctgaccccatcactctcctctttacaatgacctcccaataggttaagatgcagttattctttctcactttaagacacctttacctccggcttctgccacctcctctgctcccctgtggccactcctcacaccactccacatcccagctgttgtcaagttctttcagtgttccaaatgatctatgttctctttgcctttgagccttgcatatgttcctccctctgccagaagcgctgttccccttcctttcccacccttctgcccggccaactcaccttcattcttcccatcccagtttagaggccaccttctcgaagcctgggttggggggactcttcagtgttcccaggacaccttgtgcttcccccataatcactgggtgatcattg

SEQ ID NO: 6 - DNA, 832 bp, Homo sapiens
cccagcacacaccttgcctctggctgggaccccctttgctgctggccctgctcaggccccacagcttgatctcctcatgttcccactgctgacttccccaagctaactgtgccacagagtgggggaccccctcccggctctcacaacccccaccttcctctgcttcacttttcaccaactgaaatatggccaaaggcaaaaacccatgttcccactggcctgtgggtccccccatagatcgtaagcccaggaggaagggctgtgtttcagggctgtgatcactagcacccagaaccgtcgactggcacagaacaggcacttagggaaccctcactgaatgaatgaatgaatgaatgaatgaatgaatgtttgggcaaataaacgctgacaaggacagaagggcctagcgggaagggaacaggagtaagaccagcgcacagcccgacttgtgttcagaagacctgggattggacctgaggagttcaattttggatgaatctcttaattaacctgtggggttcccagttcctcccctgagcgcccaggacagtagagtcaacctcacgtttgagcgttggggacgcaaacacgagagtgcttggtgtgagcacacaggaggagtcacgacagagcagtgtaagagccgccacgtgggtcccacacagggggagtcacgacacagcagtgtaagagccgccacgagggtcccacacagggggagtcgcgacacagcagtgtaagagccgccacgagggtcccacacagggggagtcacgacacagcagtgtaagagccgccacgagggtcccacacagggggagtcacgacacag

SEQ ID NO: 7 - DNA, 766 bp, Homo sapiens
ttacccttggggtgggggtgtaggatgcagctggggctgcagttccaggccacggagagcctgtgaggctgggccccggggcgccctggggaggggatgcctgatggggagcctggtgggggagggtaggggagggcgggggaggacgggggagggcgccctgtgtccctgagaaggtacctggaaatgacactgctacaactcacaccacatttcaatcaaggtccataaataaaaacccattttaaatgtgccagggagcccaaggttctgagtgcccaaggaggcaccgaagacccctcctgtgggctgaaaagctcccgattatccagcctggcccacacagtcccctgtacacagggcttccgagtgcaggtcacagggaacacagactccatggtgaatgaatgaatgaatgaatgaatgaatgagggaaataagggaggaacaggccaatgggaatcaccccagagcccagataccctttgaattttgccccctatttgcccaggaccccccaccatgagctgctgctagagcctgggaagggccttggggctgcctccccaagcaggcaggctggttggggtgctgactagggcagctggggcagagggaggcaggggcaggtgggagtagggtgggggctgggtgcagcagccggggacctctggccatcttggattttttggatggatttgtttccacattccgatcgttaagattcaagatgaaacaagacacagagacccacacgacccccgag

SEQ ID NO: 8 - DNA, 751 bp, Homo sapiens
agatgatagatacatatgttagacagagataggatagatgatagatacataggttagatagagataggatagattataaatagatacacaggttagatagattagacagacagatagatacatacatagatataggatagataactagatacaatagagatagatagatagatagatagatgatagaggatagatgataaatagatatatagcttagatagagataggatagatgatagatacataggatagatagagacaggatagatgataaatagatacataggttagatagagataggacagatgataaatacataggatggatggatagatggatagatagatagatagatagatagatagatagatagatagatagacagacagacagacagacagatagatcaatccaagtcacatactgattattcttatcatccactagggctttcacatctcagccaagtcaacttggatcctctagacctgtttcttcttctggaaggtgggaactctaccttataggatcagtctgaggagttcacaaaataataagggcaaagtgcccggcacattgtaggagactagtaatgtctataaaatgaggggcttgaagtaaatgatccctctagttctctctactgctaacattctaagacctcctttacattaattgttctcaagccacatctccctcccctacaggacttctatttatttctgatcaatttcatgagtacaaataagt

SEQ ID NO: 9 - DNA, 945 bp, Homo sapiens
actgaacatttgcttttgaaatttactatctatgtaccgttggaaaatttacttaatatctctgaattttttttcttcaactgtggagtgaggaaaataatacctacttttaggtagatgatggatataacacttttctctgcatatagtagacactcagtgcataactatcgccttccttttccctctactcagaaacaaggacatctgggaccacagccacatacttacctccagtcgtttcatatcaaccaactgagctctaacatttttctgcagaagctggatatgctgtactttttctatgactttgcgcttcaggacttcaattctgcttctcagatcctctgacactcggttgtaggtattatcacggtctgaaatcgaaaatatggttattgaagtagctgctgagtgatttgtctgtaattgccagcaaaaaagaaaggaagaaaggaaggaaggagaaagaaagaaagaaagaaagaaagaaagaaagaaagaaagaaagaaagaaagaaagagaaaaaagaaagaaagaaactagcttgtaaatatgcctaattttattttggttacagtttaatctgtgagttcaaaacctatggggcatttgacttttggataatgttatgccctgcagccttccatgaatgccagttaagatgtcctaatagcaattagtaatcccaaagaaatatagaagaagaactttctttggaattttaaaggtgtaatttggagttaaaatagttggtttgattgcatttcaattattttataacatccttaatcaagggacttgaacatattggattttcttactgatgagcttttctttttaatctatagatttgaaatggttcctaagctgttttgggtcaacaggatcactcacttgccagctagtgttgcatcactgattttaaatgtcaagtgtttgtg

SEQ ID NO: 10 - DNA, 823 bp, Homo sapiens
gttggctggggctcagagagaacaaaaaggcagaggaaaaacaaatttcccctctcacttctggagatggaacacttttcttctgcttttggacatcagaaatccaagttctctggcctttggactttgggacttgtgccagcaccctcctgggttccctggcctttggcctcaaactgaaggttacactatcagcttccgttgttctaagggcttcagacttggacagccacactgccagcttccctgattcttcagcttgtagatggtctgttatgggacttttctcagtctccataaatatgtgagtcaattccccaagtgaattgccttctatctatctatctatctgtctgtctgtctgtctgtctgtctatctatctatatctatctatctatcatctatctatccatatctatctatctatctatctatctatctatctatctatctatctatcgtctatctatccagtctatctacctcctattagtctgtctctggagaacattgactaatacaacatctttaatatatcacagtttaatttcaagttatatcataccacttcatacattatataaaaccttacagtgtttctcccttctcagtgtttatggctagtaattttttactgggtgccagacactaatttttattttgctaagtggtgaatattttttatatccttaaaaatatttttgagtgttgatctgggtaaagttaagttcaatattggaaaaatattgattcttttgaggatagttatcttctaattagtctacctgttgccccataaatggcatgattttccactctgtg

SEQ ID NO: 11 - DNA, 824 bp, Homo sapiens
tactacagcaagagcgcttgaaccagatgtaggggagatagcagctggagagcataacagaggcactgacatgtgagcagctaacgaggccttttacaagacatctgtgaccacacggccaagtagaagaaagccgttaaaagcatcaaggtagttaggtaaagctgagtctgaagtaagtaaaacattgttacaggatccttggggtgtcgcttttctggccagaaacctctgtagccagtggcgcctttgcctgagttttgctcaggcccactgggctctttctgcccacacggcctggcaacttatatgtatttttgtatttcatgtgtacattcgtatctatctgtctatctatctatctatctatctatctatctatctatctatctattccccacagtgaaaataatctacaggataggtaaataaattaaggcatattcacgcaatgggatacgatacagtgatgaaaatgaactaattatagctacgtgaaactatactcatgaacacaatttggtaaaagaaactggaaacaagaatacatacggtttttgacagctgtactattttacattcccaacaacaatgcacagggtttcagtttctccacatccttgtcaacatttgttattttctgggtttttgataatagctgtgaaaggaaaataaaaacttgggccgggcgcggtggctcacgcctgtaatcccagcactttgggaggccaaggcgggcagatctcaaggtcgggagattgagaccatcctggctaacatggtgaaaacccatctctactaaaaatacaaaaacaaaaaattag

SEQ ID NO: 12 - DNA, 927 bp, Homo sapiens
catgccactaagctgtacactgaaaaacggttaacatgataaattttatgttacatacattttaccacaatttaaaaaaattattaaaaaatactaacaataggccaagcgtgatggctcacacctgtaatcccagcactttgggaggctgagacaggtggatcaattgagctcaggagtttgagaccagcctgggtaacacagtgagacccctgtctctacaaaaaaatacaaaaattagttgggcatggtggcacgtgcctgtagtctcagctacttgcagggctgaggcaggaggagttcttgagcccagaaggttaaggctgcagtgagccatgttcatgccactgcacttcactctgagtgacaaattgagaccttgtctcagaaagaaagaaagaaagaaagaaagaaagaaagaaagaaagaaagaaagaaagaaagaaagaaagaaagaaaaagagagaggaaagaaagagaaaaagaaaagaaatagtagcaactgttattgtaagacatctccacacaccagagaagttaattttaattttaacatgttaagaacagagagaagccaacatgtccaccttaggctgacggtttgtttatttgtgttgttgctggtagtcgggtttgttatttttaaagtagcttatccaatacttcattaacaatttcagtaagttatttcatctttcaacataaatacgcacaaggatttcttctggtcaagaccaaactaatattagtccatagtaggagctaatactatcacatttactaagtattctatttgcaatttgactgtagcccatagccttttgtcggctaaagtgagcttaatgctgatcaggtaaattaaaaattatagttaattaaaagggcataaatgttacctgactcaataagtcatttcaattaggtctg

SEQ ID NO: 13 - DNA, 731 bp, Homo sapiens
ctggttttggtggaattgactccctctgtcacaaactcagcttcagcccataccctgagccatagacctatccctctaatgcattgtactagtctcagggctaataacaagggagaggtgtcaaagggccagttccacctccaccaccagtggaaaagctattcccaggtgaggactgcagctgccagggcactgctccagaatgggcatgctggccatattcacttgcccacttctgcccagggatctatttttctgtggtgtgtattccctgtgcctttgggggcatctcttatactcatgaaatcaacagaggcttgcatgtatctatctgtctatctatctatctatctatctatctatctatctatctatctatctatctatctatgagacagggtcttgctctgtcacccagattggactgcagtgggggaatcatagctcactacagcctcaaactcctgggctcaagcagtcctcctgcctcagcctcccaagtacctgggattataggcatgagccaccatgtccggctaatttttttttttaagagatggggtctcgctgtgttccccagccttgtcttaaactcctggcctcaagtgatcctcccatctcagccttccaaagtgctgagattacagcagaggcttttaagtcaaagctttccctgctaggacaagccctagttaaagtcctggagcactggccactgcagctgcacttgg

SEQ ID NO: 14 - DNA, 1026 bp, Homo sapiens
cctactcgggaggctgaggcaggagaatcgcttgaacccaggagggggcgactgcagtgagccgagatcgtgccactgcactccagcctgggtgacagagcgagactccatctcaaaaaaaaaaaaaaaaaaacagaatcataggccaggcacagtggctaattgtaccttgggaggctgagacgggaggatcgagaccatcctgggcaccatagtgagaccccatctctacaaaaaaaaaaaaaaattttttttaaatagccaggcatggtgaggctgaagtaggatcacttgagcctggaaggtcgaagctgaagtgagccatgatcacaccactacactccagcctaggtgacagagcaagacaccatctcaagaaagaaaaaaaagaaagaaaagaaaagaaaagaaaagaaaagaaaagaaaagaaaagaaaagaaaagaaaagaaaagaaaaaacgaaggggaaaaaaagagaatcataaacataaatgtaaaatttctcaaaaaaatcgttatgaccataggttaggcaaatatttcttagatatcacaaaatcatgacctattaaaaaataataataaagtaagtttcatcaaaacttaaaagttctactcttcaaaagataccttataaagaaagtaaaaagacacgccacaggctaagagaaagtacttctaatcacatatctaaaaaaggacttgtgtccagattaaagaattcttacacatcaataagacaacccaattaaaaatgggcaaaagatttgaagagatatttaaccaaagaaaacatataaatgtgtccgggcgcgatggtaatcccagcactttgagaggccgaggcaggcggatcacttgaggtcaggagtttaggaccagtctggccaacatggtgaaaccctgtctctaataaaaatacaaaaattagctgggtgtggtggcgtaagcctgtaatcccagctgctcaggaggctgaggcagaagaattgcttgaacctgggaggtggaggctgcagtaagcg

SEQ ID NO: 15 - DNA, 977 bp, Homo sapiens
cacatgtggacatttcttattttctcatattggtggtatggctcatttatgaagttaatactggacattgtggggaggctgtgtaagaagtgttaaaggggatcagggatacattcacttctcttttcctttgctagttctgtggtcttaagcaaagtagcctcaaacatcagtttcctcttttataaaatgaggaaaataatactcattaccttgcatgcatgatataatgattacataacatacatgtgtgtaaagtgcttagtatcatgattgatacatggaaagaattctcttatttgggttattaattgagaaaactccttacaattttcttttcttttcttttcttttctttgagactgagtcttgctcagtcgcccaggctggagtgcaatggcgtgatctcggctcacttcaatctccacctcctgggttcaagtgattctcctgtttcagcctccagagtagctgggattacaggtgcctaccaccacacccagctaattttttgtattttagtagagacggggtttcaccatgttgcccaggctggtcttgatctcctgagctcaggtaatacacctgcatcggcctcccaaagtgctaggattgcaggcgtgaatcaccgcacctgtccacaattttcttgttattggtaccctttcatgttggtaaaatgtattttattttctcttatcaaataattttcaatgcaatgagacgtcaactttaagcccaaagtagaccagtagtaaaactaaggctgaaaccattgattgattattaccatatattgtcctaaaatattcggcttttaaaacatttggtttcatttttcatgataaaaatatgtagcatttttgcacttttaattcactttgtagagttctcaatcatttctaacacatgcttggcaatgacaagccatttgtgaaagagttttgctggctttaaaatatatgcaaatgtaatat

SEQ ID NO: 16 - DNA, 706 bp, Homo sapiens
aggtctcctcttctatacagcacatttgttcaaactaaaaacagacctcaagtatattctgcactatatagatttttttaaagtagcttcagtctcctttaatgtgaacaattgcatactgacttaatctcttcctctctcttctcttccttcactctctcccttcctctctctttctattctcctcccctcctccctgtaaaagctaccacctcatcctgggcaccctggttatatcaacttcagctatgaggtaatttttctctttactaattttgaccattgtttgcgttaacaatgccctgggctctgtaaagaatagtgtgttgattctttatcccagatgtttctcaagtggtcctgattttacagttcctaccaccagcttcccagtttaagctctgatggttggcctcaagcctgtgtcgtcccagcagcctcccgcctggccactctgactcagtctgtcctcctaaatatggccgtaagcttacccatcatgaaccactactcagggaggctccatgatagggcaaaaagtaaactctgaccagcttggttctaacccagctagtaaaatgtaaggattaggtaagatgttatttaaaactctttccagctcaaaaaactcctgattctaagatagtcacactctatgtgtgtctcttgcttgcctctgctgaaatattagtgactaagtggtata

SEQ ID NO: 17 - DNA, 712 bp, Homo sapiens
ttattctccaatattttgaaatgtgaatattacagtaatttcccttgtccaaatgagaaaaccagggttccaaagagaggaaattatttgcccaaagttagtaattttacctaatctttacattttaccggatgggatagaaccaagctggtcagtcagagttgactttttgccctttcatggaaccttcctgagcagtggttcatgaatgaataaacttacagccatatttaggaggaaagagtcaatccgaatggtcaggcaggagggtgctggagcaacacaggcttgaggccaaccatcagagcttaaactgggaagctgatggtaggaactgtaaaattgggaccacttgagaaaccactttatttgggatgaagaatccacccactattctttacagagcccaggggactgctaatgcaaacagtgatcaaaattagtaaagagaaaaattacctcatagctgaagttgatataaccagggtgcccaggatgaggtggtagcttttatagggaggaggggaggagaagagaaagagagaggaagggagagtgtgaaggaagggaagagagagtaagagattaagtcaatatgcaattgttaacattaagagagactaaaattacttttaaaaaatctatatagtacagaatatatttgaggtctgtttttcgttaaaacaagtgtgctatgtaggagaggagactt

SEQ ID NO: 18 - DNA, 840 bp, Homo sapiens
acaaggcacggaactcacacccagcctctctccatacaacagaatatgggttcttgcggagctggactctgcaggagtctatctaatatggactctgtgtcaatgactcctgggcctcctctgatcaccccattaaagtccttcgattgctttgagcctcaaatctatgtgacatcaatacgttcatttcttcctagcacttagaactgtttcttgttgatacatttgctggcttcttccctgtctcaccccttttcctaccagaatgccagtcccagaggcccttgtcagtgttcatgcctacatccctagtacctagcatggtacctgcaggtggcccataatcatgagttattcagtaagttaaaggattgcaggagggaaggaaggacggaaggaaggaaggaaggaaggaaggaaggaaggaaggaaggaaggaaggaaggcaggcaggcaggcaggcaggcaggcaaggccaagccatttctgtttccaaatccactggctccctcccacagctggattatgggccagtaggaattgccattttcagggttttgctgtcactgtagtcaggaccatgaagtctttaggcacctccactccacacaccccctggtgagagctcccatctccctgttctgaaacagctccccaatatagtactgattccggttaaacttgaacccctgcccctgcccctgcccctgatttacatgaggacactgaggcccagaggggtaaagtgactgccaggggtcacacagctagaaagtggcggtgccagaactggaaggaggccctcattcctgagtcacggcttttccatagcacagcctt

SEQ ID NO: 19 - DNA, 780 bp, Homo sapiens
atgaaactggacacagaaaccagaccccagagcacataccgtatgagtccattggtatgaagtttaaaaacagatggcactagtccaaaggattggaagttggaatagtggttaccaggactggggggaggaagggatggtggatggtgaacaaaaggaccttggagggctcctggggttctaggaatcaatcttccttctttccttccttccttccttcctctttctctctttctttctgtttttatttcaataggtttttaaggaacaggtggtgttggttacatgaataagttctttagcagtgatttctgatattttggtgcacccattacccgaataaaaatcttctctctttcttcctctctccttccttccttccttccttccttccttccttccttccttccttccttcctaccttctttccttcaacagaatcttattctgttgcccaggctggagtgcagtggtacaattatagctttttgcagcctcaacctcctgggctcaagtgatcttcctgccccagcctcctgagtagccaggactacaggaatgtgccaacatgcctggctaattttaaaaaattttttatagagaagaggtctcactatgttgcccagactagacttgaactccttccctcaagtgatctttctgcatcagtcttccaaagtgctgggattgcaggcatgagccacctcacccagccttagaaatgttctgtttcttgacctgagagctggatatacaggattgctcactttgtgaaaattc

SEQ ID NO: 20 - DNA, 887 bp, Homo sapiens
gtacttcagagtcaggatgcctctcttgctctgggcctccttgcccacataggagtccttctgacccatgcccaccatcactccctggtgcctagggtgccccacaatggaggggaagacggcctggggagccttgcgcatgctggagcagttgtcgacgacgacgagcgcggtgatagcatcatccatggtgagctggcggcgggtgcggacgcaaggcgcagcggcaaggacaaggttctgtgctcgctgggctgacgcggtctccgcggtgtaaggaggtttatatatatttctacaacatctcccctaccgctatagtaacttgctctttctttccttcctttctttctttctttctttctttctttctttctttctttctttctttctttctttctttctttttctttctttctttctttctttctttctttctttctttctctttctttctttctctttctttctttttctttctttttcttccttccttcctttctctctctctctctttctttctttctaactctctttgtctctttctttctttcttttgacggagtttcactcttgtcgcccagattggagtgcaatggcatgacctcggctcactgtagcctccacctcccaggttcaagcgattatcctgcctcagcctccctaggagctggaattacagacgtgcaccaccaagcctggctaatttttgtattattagtagagacggggtttcaccttgttggccaggctggtctcgaactcctgacctcaggtgacccacctgccttaggctcccaaagtcctgggattataggcatgagccacagtgcccagccttcttttcatttaatactatagtagtgtgatcctctctacctattaca

SEQ ID NO: 21 - DNA, 720 bp, Homo sapiens
ttctgttttgcggtggttcctagtatggtacctggccaagggcacactagatctttgtcaaggtaatgactactttttattaaatgctttccatgtatcaagttctgtgccaagcacttgacatatatcattttattttatcccgtgaagtagttattggtatcttcatttacaaataaaaaaacaagcttagtacttaactcactgccttgaacataattattgctttaaaggtagctaggattcttaatagctattattaccaaagcatgaacaatcagtaaaaagcaaacctgagcattagccccaggaccaatctggtcacaaacatattaatgaattgaacaaatgagtgagtggaaggaaggaaggaaggaaggaaggaaggaaggaaggaaggaaggaaggaaatgaagacaatacaaccagagttgttcctttaataacaagacaagggaaaaagagaactgtcagaataagtgttaattataatatccaggggtgggatacagaggttttagcatctgctctttgccaagcactgcacttattcctgaggaatacctgagggaaaaagtatggtttctcacaggatctagttggactggaaatatgacattcatattggaatccagtgtctttttctgaaaaagagagttcgttccaagcttagctcacatgcaagctaagacaaccactagaaattactctccccagggc

SEQ ID NO: 22 - DNA, 780 bp, Homo sapiens
gtcatgcctacagtgtaacgggaattgaccaggtaggcgacttgaactccaactgcaggctatggggagacatgtgacaatgctaatcccttaggcatttattcagtgcattgcagtttaaatgtctgcctttcaggcatttcagagattatgtcacctaaagaggcaggctggaattcaaaacggcaagccaggaaagagagaaaccatgtgattccaccgcagcacaaaactcgtttagcagctgtaagcgcctggtctttgtttatttttaatttcctttctttcccaattctccttcagtcctgtgttagtcaggattcttcagagaaatagaatcactagggaaccaaatatatatacatacaattaaacacacacacacctatctatctatctatctatctatctatctatctatctatctatctatctatctatctatctatctacatcacacagttgacccttgagcaacacaggcttgaacttatatggggattttcttccatctctaccacccctgagacagcaagaccaactcctcctcctccttctcagcctactcaacatgaagataataaggatgaagacctttacaatgacccagttccacttaataaatagtaaatgtatttcctcttccctatgattttcttgataacatttcttttctctggcttatttattgtaagaatacagtatataatataaataattataaaacatgttaattggttctttacgttatcgataagacttctggtcaatggtaggcta

SEQ ID NO: 23 - DNA, 780 bp, Homo sapiens
gagcccaagtttaaacccaggccctctgtgtccccctacagggtgactgcatctccgagtcctggcttgtcatgcctgacagagggctgccgagtgagcagcttaaggcatcctgccactgtgcagctgccaaccctacagcccggcagccctgcgggaggaagctctagtgcaggcctcttaggatctggggtccaggatgctgatttcagggccgggaccttgggcaccgtccctctggtctgcataagacccactatgggcaaaccttaaacctgatcgttggaattccccaaactggccagttcctctccaccctatagaccctgtcctagccttcttatagctgctatgggggctagattttccccgatgatagtagtctcattattattattattattattattattattattattattattactattattgttataaaaatattgccaatcatacattcgcgtgatcactcacactgtgccgggcactcttgagagcactttacatatattgtctcatttaattctctcaacttgggcacaggcactgtcactatttccattctacagctgaggagactgaagcacagagagccttagggacttgcctgaggtcacacagctaagaaatggtggagccaggatcagaaaccaggccacctacagagctccctgcaaggggaacagcatccggttccagaggctgtgattttatcagctacactgtgtgactccatcttcacactctcctgcccctcaagaagacatataacct

SEQ ID NO: 24 - DNA, 780 bp, Homo sapiens
atgaagagatggtcaggcgaggtatgggggaaggggcgtggagcttccatgtcctccctgggcgccaccctccaggaacctccacgtgttcagctatacagaagcttcctgaacccagtcctcttggggtttgagggaagcttcatgacatcagcattccttcctccagggtattaatgggaccctctctgaagagattcttaagacccacggccagaaagttgggtaaagactagagtcctgccttggggcaggtgaaaggagtgcaagagaaggtaagagagattctgttcctgagccctaatgcacccaacattctaacaaaaggctgtaacaagggctacaggaatcatgagccaggaactgtggctcatctatgaaaacttctatctatctatctatctatctatctatctatctatctatctatctatatcataacaccacagccacttagctccaatttaaaagattaatcataaacatttgggaaggagagtgaagatttttgtgatgttaaataagaatgattatactaaaaaccaaaataatatgttatttatggctgggtgtggtggcttaagcctgtaatcccagaactttgggaggccaaggcttgtggatcacttgagcccagaagttcaagaccagcctgggcaacatagggagaccctgtctctacaaaaaattttaaaattagctggacatgatggcacgcacccgtagtctcagctactcaggaggctcacgccactgcattccagtctgggtaacgcacac

SEQ ID NO: 25 - DNA, 780 bp, Homo sapiens
gtcaggagttcgagaccagcctggccaacatggcgaaaccctgtctctactaaaaatacaaaaaaattagctgggcatggtggtgtgttcctgtaaccccagctactcaggaggctgaggcaagagaatcgctggaacccaggaggtggaagttgcagtgagctgagattgcaccactgcactccagtgtgggcaacagagcgagactctgtctcagaaaaaaaaaagaatacatgaaatcagagaaactcaaattgtgatagtagtttcttctggtgaaggaagaaaagagaatgatatcagggaagatgaaaaaagagactgtattagtaaggcttctccagagagaaagaatcaacaggatcaatggatgcataggtagatagatagatagatagatagatagatagatagatagatagatagacagacagacagacagacagacagacagatgagaggggatttattagaggaattagctcaagtgatatggaggctgaaaaatctcatgacagtccatctgcaagctggagacccagggacactaggagcatggctcagtccaggtctaaaagccaaaaaaccagggaaactgatggtgtaattatccatcccaggtggaaggcctgagaacctggagtgcccctggtataagtcccagagtacaaagacaggagagcctggagttctgacttccaagggcagaagaatgtgtcgcagctccaggagagagagagaaagaatttctttcctccgccttttgattctatctggggg

SEQ ID NO: 26 - DNA, 74 bp, Homo sapiens
cagcaacagcacccagcttggcgcgggccgagggctcccaggcatgacactgcagatccgcgactgagcctgtg

SEQ ID NO: 27 - DNA, 82 bp, Homo sapiens
ttaagtaatgtcaagaaggcaatgcgctgagactggagagcagaagaaagcatcactgggctaacacagcaaatgtggaacc

SEQ ID NO: 28 - DNA, 84 bp, Homo sapiens
cagcctagacgtcaagttacagcccgcgcagcagcagcaaaggggaaggggcaggagccgggcacagttggatccggaggtcgt

SEQ ID NO: 29 - DNA, 91 bp, Homo sapiens
gccttcagcaggaagtccacaaccctgcaaaagagggcgctgcgtcacgcgggcacacgtccgcagtctcggagtctgtgtgaggcacagg

SEQ ID NO: 30 - DNA, 103 bp, Homo sapiens
cttctccgaggtcgcaggtggaacgggcttgcgcgtgagaacggggcctgggcttaactcactggggcctcccccgggtggccgaggttcttttcccacgccc

SEQ ID NO: 31 - DNA, 108 bp, Homo sapiens
cagcatccatcccatggtatgggtgggaagcctgaggcttgggctggtcaagggacctgcgccaggtcatgcagatgaacagcaggggagcccaagtttaaacccagg

SEQ ID NO: 32 - DNA, 480 bp, Homo sapiens
aaacaccccagtctgaaaataaccatagtttgttgctcttacgagtgaaaatgctatttcatacacgaagctttgtccttcagcacccaagatttaaggataattatggatgaatattatggattcattttaaatcctttggcaaatctgctctgggggcttctctgtcagaaggtctctccttcccaactctaagaaacgttattcctatgcaaatgctgctgagtcaagacggggagggaagtgcagagagaagggctggtggcatggtcagtaagtcatgagggtgagattaggggtgacacactgcttgccaacgtaggagaaggctctgccctcacctagcaggtctgatggaagccccttattccgtccttcctgccgggttccaccgagatccaaaaaggaatgctgtgtaggagcacatgatatgtgataaatgagagaaaggtcaaacatttaaggaacgcccagagaaag

SEQ ID NO: 33 - DNA, 540 bp, Homo sapiens
cttgaacctgagaggcagaggttgcagtgagccgagaccatgccattgcactccagcctgggcaatagagtaaaactccatcctcccgctccaaaaaagtagacaacgtccatgaggtgatgaggaaggggttatcgtgtgttgcttgctgagaacaggacccccagactcaccgtgtcgacgccggccagcagcatctcagtcacgttggcgtagatctcctgcagcgtcagagcctggctaaggaagaggtatgtgagaagtcccccgctcaccctccggcctcggtccatttggtactgtatgtccctcaacttgttgtcaacatgaatttggcctgtttgaaaacagtatttcttttgaaaggagtttgggttgagaatcatcttttcagtctcaaagccctctgtcctcccagtagcttaactaaaccagtggcaggtgacagagggtaaggaaacccaatttatctaacgtcaacctgggagtttcactcatacacttgcttatgtaaatgaatgaaaagttaaaagacaagct

SEQ ID NO: 34 - DNA, 537 bp, Homo sapiens
agagagcccaggagacaggcagaaaggaaggcatgtgaccggatcacaatcatcagctctctgctgtcctctttgggaagggttttagtattaaaaggacatttattctcattaatgcaaaattaaggagttttaaaagcttttacaacctagactccctctgagaggttagccttgacaccctaatcgccttctgctcccgccactgctcggtgccaagcagctcccacggccccggcgggtctgatgatagccggacaggagggaggaaggggaggaggaagagcctgcatcagctcctacgattgcccagccccatcctgggagtgattaaacggtgcatcaccaaatgccagtcccactgacaggcaggtcaccgtgcacttcagggcactctaaattgccgactctccatgtagagagggatgaatccaatattgaaatcctcataactacagccccccaaagtagccgtccatcttctgcttaaaatgttgatctgtagtaaaatgttgattttgttgaagctgagtgatg

SEQ ID NO: 35 - DNA, 609 bp, Homo sapiens
ttgaacctaggagatggaggttgcagtgagctgcgatcatgccacttccctccaggctgggcaacagagcgagactccatctcaaaaaaacaaaaaagaaaaccaaccttttgaatgtaggggaaacttttcaaaggatatctagttttcaattacagtaaacttgtggaagggaggttcagagttgagattgagattatagattttgctgatgataaaccatgagttccagaggacatagtagactattctgggcagttatacaggggtggatggaatgtgggagtggggttgtatagtgccataaagaaatgagagtccggattaaaaataatgagctggactcgcgagccttttgtaactgaaataaatagaaaaataagaaatacattatttctgtgattgttgagaggaagaaatggtggaaatcttgtgagaagcacactgagctctagcaccacctcttcactcctacagatggtggaataaacggcaggcaagttcaaaatcacatatagtcattattgcaagatagttctatggatatagatactacatacaatataaatcatgctcattgaatggttcagtggaaactactctgaactt

SEQ ID NO: 36 - DNA, 732 bp, Homo sapiens
gagtttgggaagggtattttaggggggaataacttttgagttcccagcgtgcgggggaagggcgggacgggagggtgtcccaaggcctgagaagatcagtgtggggcaggggtcaggaataacctgggagggggccttgtatgggggaaataattgggaagaggagagatgggatgaagggggcctcagcgggtcgtctcctgtgtatgcagggtcgttctgcagcgtctctgggagatggcgtccctgggagccctcaggtcgcccctacccgctgcggggtgctttcctggcgtcacgccttcctggcccctggagggaaggaagtgaaactctcctcttcccccacccggctggaatgcgagtcaggaagcctggggctccagcctgctccggctgcccgggtcggggatggggaggggcgtggccggagcgcaaagccccgcccctccgcgccccccccccggaagccccgccgccggccgctaaggcgatcacgggccctgtcctaatatgggcaaccggaagcggcccgcgcgactgccctacgtcactccgtccaaatttagttgtggaagtcagcgggcgctggtggcgggaaggcgccgcgagccagtgcgggcggaaagggggcggggggcgcaccaccccttaaagggcccgcaccaggaatgaatggagccattcgaacaattctgcatcctatttttggaggaagtggaattagtattt

SEQ ID NO: 37 - DNA, 488 bp, Homo sapiens
tttatttttaaaaaagaaagaaagaagagaaaagggatgggtttattgtccttttcaacagactagagtatacggggtgaaactgcttcacttgattcaataaaatcgtttccggtaacaggccccaggaatcctagacctaagcctggcgcgaaactacatttcccacaatccttcgggggctgataaggctccgcaatggtctgaactacaattcccacaatccagggcgatttccgctttgtcgcgtttcctcaaggctccgccccatttcccatctttcttttcagtccttgcgcaccggggaacaaggtcgtgaaaaaaaaggtcttggtgaggtgccgccatttcatctgtcctcattctctgcgcctttcgcagagcttccagcagcggtatgttgggccagagcatccggaggttcacaacctctgtggtccgtaggagccactatgaggagggccctgggaaggttagtgtgtaagggg

SEQ ID NO: 38 - DNA, 248 bp, Homo sapiens
accctcatttcacatttcaccccttcctcaaaatgctcccttcatattacctcctcagaaaccaagaatatggctactaattctccctggccccatgctgcaggtgaaccggtagcccagaggtatcacataattctcccaaagtcacacagcaaatcaagatgcatccaggactagaagccatgtcagccacactgggaagccccagcgaagctgacagaaagtttcataataccaccctctcccct

(19) United States
(12) Patent Application Publication
(10) Pub. No.: US 2013/0084571 A1
(43) Pub. Date: Apr. 4, 2013

(54) METHYLATION PROFILING OF DNA SAMPLES

(71) Applicants: Adam Wasserstrom, Nes-Ziona (IL); Dan Frumkin, Tel-Aviv (IL)

(72) Inventors: Adam Wasserstrom, Nes-Ziona (IL); Dan Frumkin, Tel-Aviv (IL)

(73) Assignee: Nucleix, Herzelya (IL)

(21) Appl. No.: 13/641,915

(22) PCT Filed: Apr. 19, 2011
(86) PCT No.: PCT/IB11/00861

§ 371 Date: Dec. 14, 2012

Related U.S. Application Data
(60) Provisional application No. 61/325,977, filed on Apr. 20, 2010.

(51) Int. Cl. C12Q 1/68

(52) U.S. Cl. CPC C12Q 1/68
USPC 435/6.11

(57) ABSTRACT

The present disclosure relates to methodology for fast and cost-effective identification of the source of DNA samples. DNA samples obtained from unknown or unrecognized tissues or cell types are analyzed according to the methodology described herein, yielding an identification of the tissue and/or cell type source. Identification is based on sequential biochemical procedures including methylation sensitive/dependent restriction and polymerase chain reaction, followed by analysis of the data. All biochemical steps are performed in a single test tube. The disclosure has immediate applications in forensic science for identification of the tissue source of DNA obtained from biological stains. The disclosure also has immediate applications in cancer diagnosis for identification.

### FIELD OF THE DISCLOSURE

The present disclosure embraces methodology for fast and cost-effective methylation profiling of DNA samples. Methylation profiles from DNA samples are obtained according to the methodology described herein, yielding information on the DNA sample, such as identity, physiological, and pathological characteristics.

### INTRODUCTION

Cell cultures and cell lines are important tools for conducting research in cell, tissue and organ development, studying disease, and identifying therapeutic agents. The ATCC, for instance, holds over 3,400 cell lines from over 80 species, including 950 cancer cell lines, 1,000 hybridomas, and several special collections of cells, like stem cell lines. The DSMZ-German Collection of Microorganisms and Cell Cultures also holds numerous human and animal cell lines, especially those to do with leukemia and lymphoma.

The presently described profiling methods, such as those which utilize methylation profiling, are useful for creating cell-type and cell line-specific authenticity profiles that tell a user, among other things, the functional quality and origin of cells and cell lines, and whether cells and cell lines are cross-contaminated, contaminated by microorganisms, or misidentified.

### SUMMARY

In one aspect, there is provided a method for methylation profiling of a DNA sample obtained from a cell or cell line, comprising: (a) digesting the DNA sample with a methylation-sensitive and/or methylation-dependent restriction endonuclease; (b) amplifying the digested DNA with at least a first and a second restriction locus, thereby generating an amplification product for each restriction locus; (c) determining the intensity of the signal of each amplification product; (d) calculating at least one methylation ratio between the intensity of the signals corresponding to the two restriction loci; wherein the calculated methylation ratio(s) comprise the methylation profile of the DNA sample.

In another aspect, there is provided a method for identifying the source of a DNA sample, comprising: (a) digesting the DNA sample with a methylation-sensitive and/or methylation-dependent restriction endonuclease; (b) amplifying the digested DNA with at least a first and a second restriction locus, thereby generating an amplification product for each restriction locus; (c) determining the intensity of the signal of each amplification product; (d) calculating at least one methylation ratio between the intensity of the signals corresponding to the two restriction loci; (e) comparing the methylation ratio calculated in step (d) to a set of reference methylation ratios obtained from DNA of known tissues and/or cell types; and (f) identifying the source of the DNA sample based on determining the likelihood of each tissue and/or cell type being the source of the DNA, wherein the tissue/cell type with the largest likelihood is determined to be the source of the DNA sample.

In one embodiment, the source is a tissue or cell type. In another embodiment, the source is a specific physiological/pathological condition. In another embodiment, the source is a specific age, or range of ages. In another embodiment, the source is male. In another embodiment, the source is female.

In another embodiment, the DNA digestion and amplification are performed in a single biochemical reaction in a single test tube. In a further embodiment, the single test tube comprises DNA template, digestion and amplification enzymes, buffers, primers, and accessory ingredients. In another further embodiment, the single test tube is closed and placed in a thermal cycler, where the single reaction takes place.

In another embodiment, the methylation-sensitive restriction endonuclease is unable to cut or digest DNA if its recognition sequence is methylated. In another embodiment, the methylation-sensitive restriction endonuclease is selected from the group consisting of AatII, Acc65I, AccI, AciI, AC1I, AfeI, AgeI, ApaI, ApaLI, AscI, AsiSI, AvaI, AvaII, BaeI, BanI, BbeI, BceAI, BcgI, BfuCI, BglI, BmgBI, BsaAI, BsaBI, BsaHI, BsaI, BseYI, BsiEI, BsiWI, BslI, BsmAI, BsmBI, BsmFI, BspDI, BsrBI, BsrFI, BssHII, BssKI, BstAPI, BstBI, BstUI, BstZ17I, Cac8I, ClaI, DpnI, DrdI, EaeI, EagI, Eagl-HF, EciI, EcoRI, EcoRI-HF, FauI, Fnu4HI, FseI, FspI, HaeII, HgaI, HhaI, HincII, HincII, HinfI, HinP1I, HpaI, HpaII, Hpy166ii, Hpy188iii, Hpy99I, HpyCH4IV, KasI, MluI, MmeI, MspA1I, MwoI, NaeI, NarI, NgoNIV, Nhe-HFI, NheI, NlaIV, NotI, NotI-HF, NruI, Nt.BbvCI, Nt.BsmAI, Nt.CviPII, PaeR7I, PleI, PmeI, Pm1I, PshAI, PspOMI, PvuI, RsaI, RsrII, SacII, SalI, SalI-HF, Sau3AI, Sau96I, ScrFI, SfiI, SfoI, SgrAI, SmaI, SnaBI, TfiI, TscI, TseI, TspMI, and ZraI. In a further embodiment, the methylation-sensitive restriction endonuclease is HhaI.

In another embodiment, the methylation dependent restriction endonuclease digests only methylated DNA. In a further embodiment, the methylation dependent restriction endonuclease is McrBC, McrA, or MrrA.

In another embodiment, the likelihood is determined by matching the methylation ratio of step (d) with reference ratio(s) of the same loci amplified from known tissues/cell types.

In another embodiment, the tissue and/or cell type is blood, saliva, semen, or epidermis.

In another embodiment, the restriction loci are chosen such that they produce distinct methylation ratios for specific tissues and/or cell types.

In another embodiment, the DNA sample is mammalian DNA. In a further embodiment, the mammalian DNA is DNA from a mammal selected from human, ape, monkey, rat, mouse, rabbit, cow, pig, sheep, and horse. In another further embodiment, the mammalian DNA is human DNA. In a yet further embodiment, the human DNA is from a male. In another yet further embodiment, the human DNA is from a female.

In another embodiment, the amplifying is performed using fluorescently labeled primers. In another embodiment, the signal intensity is determined by separating said amplification products by capillary electrophoresis and then quantifying fluorescence signals. In another embodiment, the amplification and determination of signal intensity are performed by real-time PCR.

There is provided a method for distinguishing between DNA samples obtained from blood, saliva, semen, and skin epidermis, comprising: (a) digesting the DNA sample with HhaI; (b) amplifying the digested DNA with forward and reverse primers for six loci set forth in SEQ ID NOs: 26-31, thereby generating an amplification product for each restriction locus; (c) determining the intensity of the signal of each amplification product; (d) calculating methylation ratios for all loci pair combinations; (e) comparing the methylation ratios calculated in step (d) to a set of reference methylation ratios obtained from DNA from blood, saliva, semen, and skin epidermis; and (f) calculating the likelihood of each of blood, saliva, semen, and skin epidermis being the source of the DNA, wherein the tissue/cell type with the largest likelihood is determined to be the source of the DNA sample.

In one embodiment, the reference methylation ratio for locus pair SEQ ID NO: 29/SEQ ID NO: 30 in blood is about 0.29. In another embodiment, the reference methylation ratio for locus pair SEQ ID NO: 29/SEQ ID NO: 30 in semen is about 2.8. In another embodiment, the reference methylation ratio for locus pair SEQ ID NO: 29/SEQ ID NO: 30 in epidermis is about 0.78.

In another aspect, there is provided a kit for determining the source of a DNA sample, wherein said kit comprises (a) a single test tube for DNA digestion and amplification using primers for specific genomic loci; and (b) instructions for calculating at least one methylation ratio and comparing it to reference methylation ratios. In one embodiment, the primers comprise forward and reverse primers for the genetic loci set forth in SEQ ID NOs: 26-31.

In another aspect, there is provided a method for determining whether a DNA sample is from blood, comprising (a) digesting the DNA sample with a methylation-sensitive and/or methylation-dependent restriction endonuclease; (b) amplifying the digested DNA with at least a first and a second restriction locus, thereby generating an amplification product for each restriction locus; (c) determining the intensity of the signal of each amplification product; (d) calculating at least one methylation ratio between the intensity of the signals corresponding to the two restriction loci; (e) comparing the methylation ratio calculated in step (d) to a set of reference methylation ratios obtained from DNA of known tissues and/or cell types; and (f) determining whether the DNA sample derives from blood based on likelihood score of blood compared with other tissue and/or cell type likelihood scores.

In another aspect, there is provided a method for determining whether a DNA sample derives from semen, comprising (a) digesting the DNA sample with a methylation-sensitive and/or methylation-dependent restriction endonuclease; (b) amplifying the digested DNA with at least a first and a second restriction locus, thereby generating an amplification product for each restriction locus; (c) determining the intensity of the signal of each amplification product; (d) calculating at least one methylation ratio between the intensity of the signals corresponding to the two restriction loci; (e) comparing the methylation ratio calculated in step (d) to a set of reference methylation ratios obtained from DNA of known tissues and/or cell types; and (f) determining whether the DNA sample derives from semen based on likelihood score of semen compared with other tissue and/or cell type likelihood scores.

In another aspect, there is provided a method for determining whether a DNA sample derives from skin epidermis, comprising (a) digesting the DNA sample with a methylation-sensitive and/or methylation-dependent restriction endonuclease; (b) amplifying the digested DNA with at least a first and a second restriction locus, thereby generating an amplification product for each restriction locus; (c) determining the intensity of the signal of each amplification product; (d) calculating at least one methylation ratio between the intensity of the signals corresponding to the two restriction loci; (e) comparing the methylation ratio calculated in step (d) to a set of reference methylation ratios obtained from DNA of known tissues and/or cell types; and (f) determining whether the DNA sample derives from skin epidermis based on likelihood score of skin epidermis compared with other tissue and/or cell type likelihood scores.

In another aspect, there is provided a A method for determining whether a DNA sample derives from saliva, comprising (a) digesting the DNA sample with a methylation-sensitive and/or methylation-dependent restriction endonuclease; (b) amplifying the digested DNA with at least a first and a second restriction locus, thereby generating an amplification product for each restriction locus; (c) determining the intensity of the signal of each amplification product; (d) calculating at least one methylation ratio between the intensity of the signals corresponding to the two restriction loci; (e) comparing the methylation ratio calculated in step (d) to a set of reference methylation ratios obtained from DNA of known tissues and/or cell types; and (f) determining whether the DNA sample derives from saliva based on likelihood score of saliva compared with other tissue and/or cell type likelihood scores.

In another aspect, there is provided a method for determining whether a DNA sample derives from urine, comprising (a) digesting the DNA sample with a methylation-sensitive and/or methylation-dependent restriction endonuclease; (b) amplifying the digested DNA with at least a first and a second restriction locus, thereby generating an amplification product for each restriction locus; (c) determining the intensity of the signal of each amplification product; (d) calculating at least one methylation ratio between the intensity of the signals corresponding to the two restriction loci; (e) comparing the methylation ratio calculated in step (d) to a set of reference methylation ratios obtained from DNA of known tissues and/or cell types; and (f) determining whether the DNA sample derives from urine based on likelihood score of saliva compared with other tissue and/or cell type likelihood scores.

In another aspect, there is provided a method for determining whether a DNA sample derives from menstrual blood, comprising (a) digesting the DNA sample with a methylation-sensitive and/or methylation-dependent restriction endonuclease; (b) amplifying the digested DNA with at least a first and a second restriction locus, thereby generating an amplification product for each restriction locus; (c) determining the intensity of the signal of each amplification product; (d) calculating at least one methylation ratio between the intensity of the signals corresponding to the two restriction loci; (e) comparing the methylation ratio calculated in step (d) to a set of reference methylation ratios obtained from DNA of known tissues and/or cell types; and (f) determining whether the DNA sample derives from menstrual blood based on likelihood score of saliva compared with other tissue and/or cell type likelihood scores.

In another aspect, there is provided a method for determining whether a DNA sample derives from vaginal tissue, comprising (a) digesting the DNA sample with a methylation-sensitive and/or methylation-dependent restriction endonuclease; (b) amplifying the digested DNA with at least a first and a second restriction locus, thereby generating an amplification product for each restriction locus; (c) determining the intensity of the signal of each amplification product; (d) calculating at least one methylation ratio between the intensity of the signals corresponding to the two restriction loci; (e) comparing the methylation ratio calculated in step (d) to a set of reference methylation ratios obtained from DNA of known tissues and/or cell types; and (f) determining whether the DNA sample derives from vaginal tissue based on likelihood score of saliva compared with other tissue and/or cell type likelihood scores.

In another aspect, there is provided a method for identifying the composition of multiple sources of a DNA sample, comprising (a) digesting the DNA sample with a methylation-sensitive and/or methylation-dependent restriction endonuclease; (b) amplifying the digested DNA with at least a first and a second restriction locus, thereby generating an amplification product for each restriction locus; (c) determining the intensity of the signal of each amplification product; (d) calculating at least one methylation ratio between the intensity of the signals corresponding to the two restriction loci; (e) comparing the methylation ratio calculated in step (d) to a set of reference methylation ratios obtained from DNA of known tissues and/or cell types; (f) determining the likelihood of each tissue and/or cell type contributing to the source of DNA; and (g) determining the composition of the source DNA based on the likelihoods obtained in step (f). In one embodiment, the DNA sample comprises a mixture of DNA from more than one of blood, semen, saliva, skin epidermis, urine, menstrual blood, vaginal tissue.

### BRIEF DESCRIPTION OF THE DRAWINGS

FIG. 1: schematic overview for determining a single methylation ratio. The source of the DNA to be digested as indicated can be isolated from a cell or cell line whose identity, functionality, authenticity, origin, or contamination status, for instance, is being evaluated.

FIG. 2: schematic details for determining a single methylation ratio. The source of the DNA to be digested as indicated can be isolated from a cell or cell line whose identity, authenticity, origin, or contamination status, for instance, is being evaluated.

FIG. 3: Methylation ratios in semen and blood DNA samples in a specific pair of loci. In semen, the methylation ratio is about 2.5, while in blood the methylation ratio is about 0.25. Numbers next to each peak are the relative fluorescence units (rfu) level of that peak. Notice that the methylation ratio is independent of the absolute rfu levels.

FIG. 4: Normalization of methylation ratios. The top and bottom panels represent two channels of a single electropherogram. Signals in the lower channel were used for obtaining a linear fit (grey line). For the two loci in the top panel, a non-normalized methylation ratio (MR) was calculated by dividing the respective rfus. A normalized methylation ratio was also calculated for the loci in the top panel by multiplying the non-normalized methylation ratio by the reciprocal of a corresponding ratio obtained from the loci's projections on the linear fit.

FIG. 5: Combined tissue identification and DNA profiling of a DNA sample from skin epidermis. Peaks corresponding to loci used for tissue identification are found in the range of <110 bps (top and middle panels), while other peaks correspond to loci used for DNA profiling.

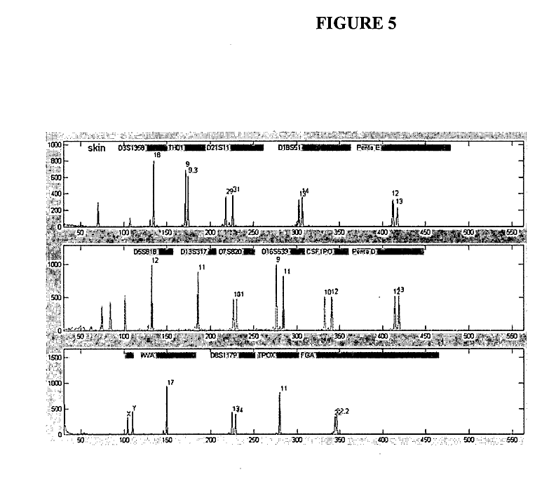

FIG. 6: Electropherograms of capillary electrophoresis of nine DNA samples extracted from semen, blood, and epidermis from three individuals. Differential methylation in semen, blood, and epidermis is evidenced by the different intensities of the analyzed loci.

FIG. 7: Electropherograms of capillary electrophoresis of eleven DNA samples extracted from blood, saliva, skin, semen, menstrual blood, vaginal tissue, and urine. Differential methylation in blood, saliva, skin, semen, menstrual blood, vaginal tissue, and urine is evidenced by the different intensities of the analyzed loci.

### DETAILED DESCRIPTION

The present disclosure relates to methylation profiling methods useful for creating cell-type and cell line-specific “functionality” profiles that tell a user, among other things, whether the functional aspects of the cell are the same or different than another cell of the same type. This particular use of the inventive methylation profiling technique is helpful because it provides information about a particular cell sample that cannot otherwise be obtained or inferred from existing and conventional cell profiling techniques.

This methylation profiling technique makes use of another inventive aspect of the technology which is the identification of loci throughout genomic regions that are methylated, unmethylated, and partially methylated. This collection of loci, whose individual methylated locus status is now known, is useful for investigating and profiling the methylation status of any cell sample. By creating corresponding methylation profiles of a cell sample, as described herein, one can determine whether cells from the sample are functioning the same way as normal, healthy cells, i.e., they exhibit a normal methylation profile, or they exhibit a different, perhaps abnormal methylation profile, compared to a known sample of the same kind of cell or cell type. Likewise, one can determine whether cells from the sample are functioning the same way as normal, healthy cells from a particular organ or tissue, i.e., they exhibit an organ- or tissue-specific methylation profile. Thus, the inventive methylation profiling techniques lend themselves to the determination of the pathogenic or physiological status of a particular cell sample.

Specifically, the inventive methylation ratios described herein are calculated from comparative analysis of the methylation status of any number of genomic loci and are useful for creating cellular methylation profiles for determining cellular origin, functional identity, age-identification, physiological profiling, and pathological status of a cell sample. Furthermore, in each instance, the methylation profiling technique can also be used to ascertain whether the obtained methylation profile reflects the presence of contaminating cells, either from, for instance, another cell line, or microbial growth, and whether a particular cell sample has been misidentified.

A methylation profiling of a cell or cell line can be readily obtained by the present invention, for example, by (a) isolating DNA from a cell sample and digesting it with a methylation-sensitive and/or methylation-dependent restriction endonuclease; (b) amplifying the digested cellular DNA with at least a first and a second restriction locus, thereby generating an amplification product for each restriction locus; (c) determining the intensity of the signal of each amplification product; and then (d) calculating at least one methylation ratio between the intensity of the signals corresponding to the two restriction loci. The calculated methylation ratio(s) is an example of the methylation profile of the DNA sample obtained from that cell sample.

By comparing the profile to a known cell of the same origin and species, or from an uncontaminated corresponding cell line, it is possible to determine the identity of the cell sample and whether or not it is, for instance, functionally similar or identical to the known cell based on its methylation profile. Accordingly, one may either commercially purchase, or create, or modify a human liver cell line and then use the present cellular methylation profiling techniques described herein to determine the functional characteristics of the cell line, in comparison to a known liver cell reference profile.

In this respect, the inventive cellular methylation profiling methods have several advantages over existing cell identification techniques, as described below. Methylation in the human genome occurs in the form of 5-methyl cytosine and is confined to cytosine residues that are part of the sequence CG (cytosine residues that are part of other sequences are not methylated). Some CG dinucleotides in the human genome are methylated, and others are not. Methylation is cell and tissue specific, such that a specific CG dinucleotide can be methylated in a certain cell and, at the same time, unmethylated in a different cell, or methylated in a certain tissue and, at the same time, unmethylated in different tissues. Since methylation at a specific locus can vary from cell to cell, when analyzing the methylation status of DNA extracted from a plurality of cells, e.g. from a forensic sample, the signal can be mixed, showing both the methylated and unmethylated signals in varying ratios. Various data sources are available for retrieving or storing DNA methylation data and making these data readily available to the public, for example “DNA Methylation Database” (MetDB) (www.methdb.net).

The inventive cellular methylation profiling methods are advantagous over existing cell profiling techniques because they minimize and effectively eliminate problems inherent with conventional profiling regimes. First, as mentioned above, the methylation profiling technique does not rely on determining levels of methylated loci but rather utilizes the inventive concept of creating methylation ratios between two genomic loci. Accordingly, unlike the prior art methods, the cellular methylation profile described herein is not limited by sample size or subject to differences in amounts or quantities of samples analyzed.

Thus, secondly, the methylation profile can be compared to the methylation profiles of reference cells to help verify the originating identity of the cell or cell line. For example, if two cell lines are obtained from the same individual, conventional DNA profiling cannot distinguish between them. But the cellular methylation profiling technique of the present invention can differentiate between the two cell types if they are obtained from different tissues or at different time points from that individual.

Thirdly, the inventive cellular methylation profiling techniques can be used to establish the functional identity of a cell line. Thus, it can be used, for example, to determine whether a certain candidate cell line is appropriate for use as a model cell line for liver because the techniques make it possible to determine whether the cellular methylation profile of the candidate cell line is consistent with the cellular methylation profile of liver.

Fourth, the cellular methylation profile is useful for determining the age of a DNA sample, because the cellular methylation profile changes with age.

Fifth, the cellular methylation profile is useful for determining the physiological state of the cell or cell line. For example, the methylation profile can indicate at what stage of the menstrual cycle cells and DNA samples were obtained from an individual.

Sixth, and as described herein, the cellular methylation profile can be used in pathological analyses, for instance to identify cellular and tissue changes that occur when a tissue is subjected to various stress factors such as inflammation, and also when inflicted by diseases such as cancer.

Thus, the uses to which the inventive methylation ratios calculated from comparisons of the methylation status of any number of genomic loci can be put are numerous, as exemplified above, such as, but not limited to, the use of a cellular methylation profile to determine cellular origin, functional identity, age-identification, physiological profiling, and pathological status. The methylation profiling technique can also be used to ascertain whether the obtained methylation profile reflects the presence of contaminating cells, either from, for instance, another cell line, or because of undesirable microbial growth.

An added advantage of the present methylation profiling methods is that, in contrast to conventional methylation analysis methods, which determine the actual methylation levels at specific genomic loci, the methodology described herein does not rely on such determination of levels which are often highly variable between different individuals. Instead, the inventive assays make it possible to use methylation ratios as indicators of the functional attributes of a cell type or cell line, and to also help identify the source, quality, and contamination status of the cell sample, even though the cells' actual methylation levels between genomic loci are variable.

An underlying aspect of the present cellular methylation profiling assay therefore is the comparison of signals from at least two loci amplified from a digested sample of DNA obtained from a cell, which ultimately yields a numerical ratio. This ratio can then be compared to reference ratio values of a pure and uncontaminated cell of the same type and species as the tested cell.

Thus, the present technology contemplates, in one embodiment, (1) obtaining DNA from one or more cells from a cell culture or cell line, (2) digesting the cellular DNA with a methylation-sensitive and/or methylation-dependent enzyme, (3) PCR amplifying the digested DNA with locus-specific primers, and (4) measuring the intensity of the signals from locus-specific amplification products; and determination of a methylation ratio. If the numerical ratio between the two amplification products matches or approximates that of a reference ratio of the same loci amplified from a known reference cell, then a conclusion can be drawn about the functional authenticity of the cell sample or, for instance, whether the sample of cells or the cell line is contaminated by some other cellular source that alters the methylation profile of the sample.

The technique may further comprise comparing the methylation profile of a cell sample with the known methylation profile of at least one cellular reference and determining whether the similarities or differences in the profiles indicates the functional, physiological, or pathological identity of the cell sample. By cellular reference is meant either the methylation profile of a known and equivalent cell type, e.g., liver, brain, lung, ovary, against which the cell sample's methylation profile can be directly compared; or a cellular reference may comprise a library of known methylation profiles from a range of different species, organs, or pathological disease states, such as cancer, and subsequently identifying to which methylation profile the cell sample most closely resembles. Thus, if a cell line is obtained and purported to be a human liver cell line, for instance, then the present technique makes it possible to compare the methylation profile of that human liver cell line against a known human liver cell line to confirm or verify the identity, or functional identity, of the obtained human liver cell line. Alternatively, one or more methylation profiles of a cell sample of unknown source can be obtained and compared against a library of known methylation profiles from different species, organs, or pathological disease states to determine its origin.

As used herein, any type of cell, such as, but not limited to, a cell from a mammal, fish, reptile, bird, bacteria, microorganism, amphibian, insect, fungi, virus, plant, of crop, can be analyzed according to the present inventive technology. The present cellular profiling techniques are therefore useful for authenticating the functional identity of, for instance, human cells, rat cells, mouse cells, monkey cells, primate cells, zebrafish cells, dog cells, cat cells, cattle cells, rabbit cells, hamster cells. The cellular profiling techniques also are useful for confirming or verifying the authenticity organ specific cell types, such as, but not limited to, the functional authenticity of liver cells, kidney cells, pancreatic cells, lung cells, cardiac cells, ovary cells, bone marrow, brain cells, breast cells, tongue cells, retinal cells, colon cells, cervical cells, embryo cells, and skin cells. The cellular profiling techniques also are useful for confirming the disease or cancer identity of particular cells, such as, but not limited to, melanoma cells, glioblastoma cells, leukemia cells, B lymphoma cells, head and neck carcinoma cells, neuroblastoma cells, adenocarcinoma cells, metastatic lymph node cells, hepatoma cells, T-cell leukemia cells, lymphoblastoid cells, breast cancer cells, cervical cancer cells, and other types of cancer cells and cell lines.

In this regard, the use of the words cell, cell culture, and cell line are interchangeable with respect to the descriptions of various profiling methods described herein. Cells that are cultured directly from an individual are primary cells, which typically stop dividing after passage of a certain number of population doublings. An established or immortalized cell line is one that can proliferate indefinitely. The inventive cellular methylation profiling techniques can be used to confirm the functional identity, physiological or pathogenic status, authenticity, tissue origin, and contamination status of any of such isolated cells and cell lines. Accordingly, it should be understood that reference in this disclosure to a cell or to a cell line is not limiting and is not meant to exclude the use of the described technique on other cells or cell lines.

Examples of common cell lines include but are not limited to human DU145 (Prostate cancer), human Lncap (Prostate cancer), human MCF-7 (breast cancer), human MDA-MB-438 (breast cancer), human PC3 (Prostate cancer), human T47D (breast cancer), human THP-1 (acute myeloid leukemia), human U87 (glioblastoma), human SHSY5Y Human neuroblastoma cells, human Saos-2 cells (bone cancer); primate Vero (African green monkey Chlorocebus kidney epithelial cell line initiated 1962); rat tumor cell lines, such as GH3 (pituitary tumor) and PC12 (pheochromocytoma); mouse cell lines, such as MC3T3 (embryonic calvarial); plant cell lines, such as Tobacco BY-2 cells; and other cells, such as zebrafish ZF4 and AB9 cells, Madin-Darby Canine Kidney (MDCK) epithelial cell line, and Xenopus A6 kidney epithelial cells. Examples of the types of tumor cell lines that can be profiled according to the present methylation profiling techniques can be found, for instance, at the ATCC's website at atcc.org/Portals/1/TumorLines.pdf, the DSMZ website at dsmz.de/human_and_animal_cell_lines/cell_line_index.php, and at the EMBL-ESTDAB database at ebi.ac.uk/ipd/estdab/directory.html.

Another problem with these, and other, cell lines is that they can become contaminated, such as by the growth of unrelated cells, cross-contaminated by other cell lines, or contaminated by microbes. See Drexler et al., Leukemia, 13, pp. 1601-1607 (1999), Drexler et al., Blood, 98(12), pp. 3495-3496 (2001), and Cabrera et al., Cytotechnology, 51(2), pp. 45-50 (2006). Furthermore, another problem is that sometimes cell lines can be falsely or incorrectly identified, which can lead to issues in interpreting results from experiments and data. The present methylation profiling methods can be used, as described herein, also to ascertain the contamination status of a cell sample.

The assays described herein are therefore powerful, multiplex, accurate, and inexpensive techniques applicable in any setting that calls for the identification and functional characterization of cells and cell lines, as well the verification of a source of a cellular or DNA sample. Thus, the assays can be used for a large number of purposes, including but not limited to the police in a forensics capacity; the health care industry for diagnostic and therapeutic purposes; in the insurance industry to verify claims pursuant to anti-discrimination genetic laws, such as the Genetic Information Nondiscrimination Act (H.R. 493); by prosecutors and defense counsel for evidentiary purposes in criminal trials and civil proceedings and appeals; and the food and agriculture industry to verify the integrity of meats, crops, and plants such as grapevines and sources of coffee. The present technology is not limited to these non-exclusive, but representative, applications.

A significant aspect of the present disclosure is that it can readily complement and expand the usefulness of existing commercial DNA profiling kits to do more than profile a particular subject's DNA. The combination of the assays disclosed herein, such as the methylation ratio assay described in detail below, with Promega Corporation's PowerPlex® 16 kit, for example, enables one to not only profile an individual's DNA composition but also to determine the source of that individual's DNA. For example, and in no way limiting, the present technology enables one to determine if a DNA sample derives from a particular tissue and/or cell type, such as blood, saliva, or semen.

Specific compositions, methods, and/or embodiments discussed herein are merely illustrative of the present technology. Variations on these compositions, methods, or embodiments are readily apparent to a person of ordinary skill in the art, based upon the teachings of this specification, and are therefore included as part of the disclosure.

The present technology uses many conventional techniques in molecular biology and recombinant DNA. These techniques are explained in, e.g., Current Protocols in Molecular Biology, Vols. I-III, Ausubel, Ed. (1997); Sambrook et al., Molecular Cloning: A Laboratory Manual, Second Ed. (Cold Spring Harbor Laboratory Press, Cold Spring Harbor, N.Y., 1989); DNA Cloning: A Practical Approach, Vols. I and II, Glover, Ed. (1985); Oligonucleotide Synthesis, Gait, Ed. (1984); Nucleic Acid Hybridization, Hames & Higgins, Eds. (1985); Transcription and Translation, Hames & Higgins, Eds. (1984); Perbal, A Practical Guide to Molecular Cloning; the series, Meth. Enzymol., (Academic Press, Inc., 1984); Gene Transfer Vectors for Mammalian Cells, Miller & Calos, Eds. (Cold Spring Harbor Laboratory, NY, 1987); and Meth. Enzymol., Vols. 154 and 155, Wu & Grossman, and Wu, Eds., respectively.

### Definitions

In describing the present technology, numerous technical terms are used. Unless defined otherwise, all technical and scientific terms used herein generally have the same meaning as commonly understood by one of ordinary skill in the art to which this technology belongs. As used herein, unless otherwise stated, the singular forms “a,” “an,” and “the” include plural reference. Thus, for example, a reference to “a nucleic acid” is a reference to one or more nucleic acids.

As used herein, the term “allele” is intended to be a genetic variation associated with a segment of DNA, i.e., one of two or more alternate forms of a DNA sequence occupying the same locus.

The term “biological sample” or “test sample” as used herein, refers to, but is not limited to, any biological sample derived from a subject. The sample suitably contains nucleic acids. In some embodiments, samples are not directly retrieved from the subject, but are collected from the environment, e.g. a crime scene or a rape victim. Examples of such samples include fluids, tissues, cell samples, organs, biopsies, etc. Suitable samples are blood, plasma, saliva, urine, sperm, hair, etc. The biological sample can also be blood drops, dried blood stains, dried saliva stains, dried underwear stains (e.g. stains on underwear, pads, tampons, diapers), clothing, dental floss, ear wax, electric razor clippings, gum, hair, licked envelope, nails, paraffin embedded tissue, post mortem tissue, razors, teeth, toothbrush, toothpick, dried umbilical cord. Genomic DNA can be extracted from such samples according to methods known in the art.

The terms “capillary electrophoresis histogram” or “electropherogram” as used herein refer to a histogram obtained from capillary electrophoresis of PCR products wherein the products were amplified from genomic loci with fluorescent primers.

The term “methylated” as used herein means methylated at a level of at least 80% (i.e. at least 80% of the DNA molecules methylated) in DNA of cells of tissues including blood, saliva, semen, epidermis, nasal discharge, buccal cells, hair, nail clippings, menstrual excretion, vaginal cells, urine, and feces.

The term “partially-methylated” as used herein means methylated at a level between 20-80% (i.e. between 20-80% of the DNA molecules methylated) in DNA of cells of tissues including blood, saliva, semen, epidermis, nasal discharge, buccal cells, hair, nail clippings, menstrual excretion, vaginal cells, urine, and feces.

The term “unmethylated” as used herein means methylated at a level less than 20% (i.e. less than 20% of the DNA molecules methylated) in DNA of cells of tissues including blood, saliva, semen, epidermis, nasal discharge, buccal cells, hair, nail clippings, menstrual excretion, vaginal cells, urine, bone, and feces. The methods provided herein have been demonstrated to distinguish methylated and unmethylated forms of nucleic acid loci in various tissues and cell types including blood, saliva, semen, epidermis, nasal discharge, buccal cells, hair, nail clippings, menstrual excretion, vaginal cells, urine, bone, and feces.

The terms “determining,” “measuring,” “assessing,” “assaying”, and “evaluating” are used interchangeably to refer to any form of quantitative or qualitative measurement, and include determining if a characteristic, trait, or feature is present or not. Assessing may be relative or absolute. “Assessing the presence of” includes determining the amount of something present, as well as determining whether it is present or absent.

The term “forensics” or “forensic science” as used herein refers to the application of a broad spectrum of methods aimed to answer questions of identity being of interest to the legal system. For example, the identification of potential suspects whose DNA may match evidence left at crime scenes, the exoneration of persons wrongly accused of crimes, identification of crime and catastrophe victims, or establishment of paternity and other family relationships.

The term “locus” (plural—loci) refers to a position on a chromosome of a gene or other genetic element. Locus may also mean the DNA at that position. A variant of the DNA sequence at a given locus is called an allele. Alleles of a locus are located at identical sites on homologous chromosomes. A control locus is a locus that is not part of the profile. A control locus can simultaneously be a restriction locus as can the profile locus. A restriction locus is a locus that comprises the restriction enzyme recognition sequence that is amplified and subsequently part of the locus amplicon. The term “natural DNA” or “natural nucleic acid” as used herein refers to, but is not limited to, nucleic acid which originates directly from the cells of a subject without modification or amplification.

The term “nucleic acid” as used herein refers to, but is not limited to, genomic DNA, cDNA, hnRNA, mRNA, rRNA, tRNA, fragmented nucleic acid, and nucleic acid obtained from subcellular organelles such as mitochondria. In addition, nucleic acids include, but are not limited to, synthetic nucleic acids or in vitro transcription products.

The term “nucleic-acid based analysis procedures” as used herein refers to any identification procedure which is based on the analysis of nucleic acids, e.g. DNA profiling.

The term “STR primers” as used herein refers to any commercially available or made-in-the-lab nucleotide primers that can be used to amplify a target nucleic acid sequence from a biological sample by PCR. There are approximately 1.5 million non-CODIS STR loci. Non-limiting examples of the above are presented in the following website www.cstl.nist.gov/biotech/strbase/str_ref.htm that currently contains 3156 references for STRs employed in science, forensics and beyond. In addition to published primer sequences, STR primers may be obtained from commercial kits for amplification of hundreds of STR loci (for example, ABI Prism Linkage Mapping Set-MD10 -Applied Biosystems), and for amplification of thousands of SNP loci (for example, Illumina BeadArray linkage mapping panel). The term “CODIS STR primers” as used herein refers to STR primers that are designed to amplify any of the thirteen core STR loci designated by the FBI's “Combined DNA Index System”, specifically, the repeated sequences of TH01, TPDX, CSF1PO, VWA, FGA, D3S1358, D5S818, D7S820, D13S317, D16S539, D8S1179, D18S51, and D21S11, and the Amelogenin locus.

“Intensity of signal” refers to the intensity and/or amount of signal corresponding to amplification products of a genomic locus. For example, in capillary electrophoresis the intensity of signal of a specific locus is the number of relative fluorescence units (rfus) of its corresponding peak.

Methylation Ratio (also called “Observed Methylation Ratio”) refers to relative signal intensities between a pair of loci. A methylation ratio is calculated by dividing the intensity of signal of the first locus in the locus pair by the intensity of signal of the second locus in the pair. In case that the intensity of signal of the second locus in the pair is zero, it is assigned an arbitrary small intensity signal (in order to avoid division by zero). Unless indicated otherwise, methylation ratios are calculated from DNA samples of unknown origin.

Reference Methylation Ratios (also called “Empirical Methylation Ratios”) are methylation ratios obtained from samples of DNA of known sources, also called reference DNAs. Similar to methylation ratios, reference methylation ratios can be determined, for example, by dividing the intensity of signal of the first locus in the locus pair by the intensity of signal of the second locus in the pair. Because reference methylation ratios are determined from DNA of known source, one can create a library of known ratios between various pairs of genomic loci.

Probability Scores are calculated by comparing observed methylation ratios to reference methylation ratios. The probability score of a certain DNA sample at a certain methylation ratio and for a certain category (e.g. blood), provide a measure of the likelihood that the DNA sample originated from that category, based on the relative position of the observed methylation ratio to the distribution of reference methylation ratios of that category.

Combined Probability Scores (CPS) of each tissue/cell type can be calculated from the single probability scores, for example by calculating the nth root of the product of the single probability scores (where n is the number of methylation ratios).

Likelihood: For each tissue/cell type, a Likelihood Score (LS) represents the likelihood that the DNA sample originated from that tissue/cell type. Likelihood scores for each tissue/cell type can be calculated for example as follows:

LS(tissue)=CPS(tissue)/[sum of CPSs of all tissues].

A. Selection and Isolation of DNA Sample

In one aspect, the present disclosure provides methodology for determining the tissue/cell type source of a DNA sample. For example, a DNA sample of unknown origin undergoes a procedure including one or more biochemical steps followed by signal detection. Following signal detection, the signal is analyzed to determine the source of the DNA sample. These methods are employed on any DNA sample in question, including but not limited to DNA from a body fluid stain found at a crime scene, or DNA from cancerous lesions of unknown origin.

The isolation of nucleic acids (e.g. DNA) from a biological sample may be achieved by various methods known in the art (e.g. see Sambrook et al, (1989) Molecular Cloning: A Laboratory Manual, 2nd ed. Cold Spring Harbor, N.Y.). Determining the source of the DNA sample may be accomplished using various strategies, including those described in the following sections.

The present inventors discovered that methylation ratio profiles can be used to determine the source of a DNA sample.

B. Methodology for Determining Methylation Levels of Genomic Loci

There are several different methods for determining the methylation level of genomic loci. Examples of methods that are commonly used are bisulfite sequencing, methylation-specific PCR, and methylation-sensitive endonuclease digestion.

Bisulfite sequencing. Bisulfite sequencing is the sequencing of bisulfite treated-DNA to determine its pattern of methylation. The method is based on the fact that treatment of DNA with sodium bisulfite results in conversion of non-methylated cytosine residues to uracil, while leaving the methylated cytosine residues unaffected. Following conversion by sodium bisulfite, specific regions of the DNA are amplified by PCR, and the PCR products are sequenced. Since in the polymerase chain reaction uracil residues are amplified as if they were thymine residues, unmethylated cytosine residues in the original DNA appear as thymine residues in the sequenced PCR product, whereas methylated cytosine residues in the original DNA appear as cytosine residues in the sequenced PCR product.

Methylation-specific PCR: Methylation specific PCR is a method of methylation analysis that, like bisulfite sequencing, is also performed on bisulfite-treated DNA, but avoids the need to sequence the genomic region of interest. Instead, the selected region in the bisulfite-treated DNA is amplified by PCR using two sets of primers that are designed to anneal to the same genomic targets. The primer pairs are designed to be “methylated-specific” by including sequences complementing only unconverted 5-methylcytosines, or conversely “unmethylated-specific”, complementing thymines converted from unmethylated cytosines. Methylation is determined by the relative efficiency of the different primer pairs in achieving amplification.

It should be understood in the context of the present disclosure that methylation-specific PCR determines the methylation level of CG dinucleotides in the primer sequences only, and not in the entire genomic region that is amplified by PCR. Therefore, CG dinucleotides that are found in the amplified sequence but are not in the primer sequences are not included in the CG locus.

Methylation-sensitive endonuclease digestion: Digestion of DNA with methylation-sensitive endonucleases represents a method for methylation analysis that can be applied directly to genomic DNA without the need to perform bisulfite conversion. The method is based on the fact that methylation-sensitive endonucleases digest only unmethylated DNA, while leaving methylated DNA intact. Following digestion, the DNA can be analyzed for methylation level by a variety of methods, including gel electrophoresis, and PCR amplification of specific loci.

In methylation-sensitive endonuclease digestion, each CG locus is comprised of one or more CG dinucleotides that are part of recognition sequence(s) of the methylation-sensitive restriction endonuclease(s) that are used in the procedure. CG dinucleotides that are found in the amplified genomic region, but are not in the recognition sequence(s) of the endonuclease(s) are not included in the CG locus.

In one embodiment, the one or more CG loci that are detected are partially methylated in natural DNA, but would be unmethylated in artificial DNA. Partial methylation would be expected to result in a mixture of T and C at the position being interrogated. Hybridization would be observed to both the T specific probes/primers and the C specific probes/primers, similar to detection of a heterozygous SNP. Relative amounts of hybridization may be used to determine the relative amount of methylation. Alternatively, both C and T would be observed upon bisulfite sequencing. Alternatively, fluorescent signals corresponding to amplification products of methylated or partially methylated CG loci can be detected.

C. Methylation Ratio Assay

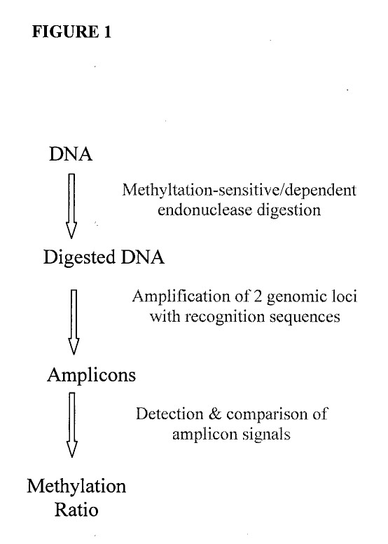

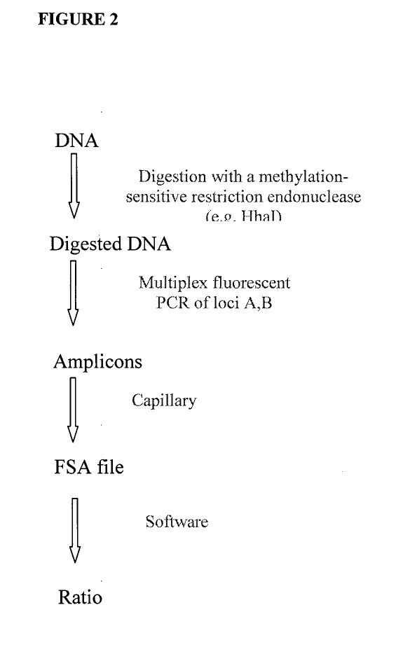

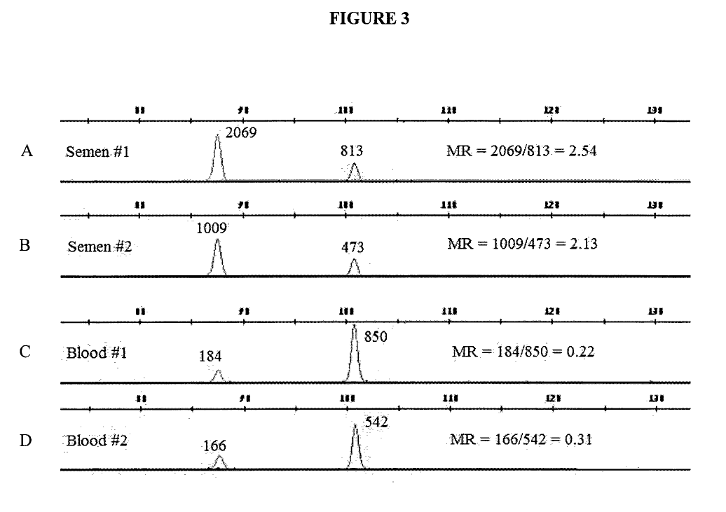

As mentioned above, one particular assay of the present disclosure involves the quantitative comparison of intensity of the signals from a pair of locus-specific amplification products produced by performing a Polymerase Chain Reaction on restriction-digested DNA. See, e.g., FIGS. 1 and 2. The numerical ratio of intensities allows one to identify the tissue/cell type source of the DNA sample. For example, in one embodiment, locus 1 and locus 2 can be amplified using fluorescently labeled primers, separated by electrophoresis, and the intensity of the signals is the relative fluorescence units (rfu) of peaks corresponding to the loci. See, e.g., FIG. 3. The intensity of the signals will correspond to the successfulness of amplification of locus 1 and locus 2 from the source DNA template. By comparing rfu between the two amplification products one can calculate a ratio that reflects whether there is more or less of one amplification product than another.

In addition, however, one aspect of this assay includes the predetermination of the expected methylation ratios from various types of tissues/cell types. Thus, the template DNA that is subject to analysis is first digested with a methylation-sensitive restriction endonuclease before it is cycled through the PCR amplification protocol. It is not necessary for both primer pairs to have a similar amplification efficiency, nor is it necessary to have knowledge of the absolute methylation levels. In order to be able to correlate an observed methylation ratio with a specific tissue/cell type, one of ordinary skill in the art may compare the observed ratio with ratios obtained empirically from DNA samples of known origin.

With this premise, the present assays comprise digesting a DNA sample with a methylation-sensitive and/or methylation-dependent enzyme, performing a PCR amplification reaction on the digested DNA, and determining the intensity of the signals from locus-specific amplification products. As mentioned, the intensity of signals can be quantified or measured by using fluorescent PCR. If the numerical ratio between the two amplification products matches or approximates that of the reference ratio of the same loci amplified from a known tissue/cell type, then the test DNA sample is determined to be of that tissue/cell type.

This particular methylation ratio assay does not depend upon identifying or obtaining measurements of the absolute methylation fraction or level of selected loci. In addition, this particular methylation ratio assay does not depend upon the efficiencies of the primer pairs used, does not necessitate that both primer pairs have similar efficiencies, is not reliant upon amount of input template DNA, is not reliant upon specific thermocycler machine and reaction conditions. Rather, the assay determines the ratio between two signals which correspond to the ratio of methylation levels in the different loci. By this manner, the quantity or concentration of starting DNA material in the sample is irrelevant to the analysis and does not skew the output results. That is, the ratio of signal levels between a first locus and a second locus will remain constant regardless of how much DNA is used as a template for PCR and regardless of the number of amplification cycles that are run on the PCR thermocycler. For example, a methylation ratio of 10 between loci 1 and 2 will remain the same whether the input DNA represents methylation levels of 0.9 and 0.09 (90% methylation in locus 1 and 9% in 2), or 0.5 and 0.05 (50% methylation in locus 1 and 5% in 2), etc.

The methylation ratio assay of the present disclosure has several advantages over other approaches for analyzing methylation. For instance, this assay is insensitive to various “noise” factors inherent when relying on the absolute quantification of methylation level, since such quantification is sensitive to noise and fluctuates as a consequence of changes in template DNA concentration, thermocycler manufacturer, PCR conditions, and presence of inhibitors. Instead, the presently-calculated methylation ratios are insensitive to such factors, since the analyzed loci are co-amplified in the same reaction and are therefore subject to the effects of such disparities. Thus, the present methodology does not require absolute quantification of genomic targets or amplicons; and the assay is a single stand-alone reaction with no need for a standard curve or any external controls.

The methylation ratio assay can be performed on very small quantities of DNA in a single biochemical reaction and is therefore an inexpensive, rapid, and powerful method for establishing, for example, the tissue/cell type source of a DNA sample. An important feature of the design of the present methods is that it can be combined with other PCR-based procedures, such as DNA profiling, in a single biochemical reaction.

In addition, the assay can detect useful biological information and can perform the task of identifying the source of DNA when simple determination of actual methylation levels fails. The assay relies on methylation ratios between samples, which are relatively constant between different individuals, and does not rely on actual methylation levels of any specific locus, which vary very significantly between different individuals.

This assay therefore provides a useful biochemical marker in the form of, in one example, a numerical ratio, that can be used to differentiate between different sources of DNA. More particular details of this exemplary assay follow.

(1). Primers For Locus-Specific Amplification

Accordingly, an aspect of the present disclosure concerns obtaining a “methylation ratio” (MR) in which the intensities of signals of amplification products of DNA loci produced from fluorescent PCR are compared to one another in order to calculate ratios between pairs of loci, e.g., Loci #1 vs. Loci #2; Loci #1 vs. Loci #3; Loci #1 vs. Loci #4; Loci #2 vs. Loci #3, Loci #2 vs. Loci #4, and so on. When this technique is used to determine the source of a DNA sample, the primers that are used in the methylation ratio amplification reactions are chosen so as to amplify a pair of loci that are differentially methylated in various tissues/cell types.

One consideration for selecting which two pairs of primers (a first pair and a second pair) to use to amplify two loci (1) and (2) is the degree to which the two loci are differentially methylated in various tissues/cell types. Thus, for example, a pair of loci whose methylation ratio is greater than 1 in one tissue/cell type, and less than 1 is all other tissues/cell types can be used to design primers for the methylation ratio amplification assay.

(2) Selection of Loci For Amplification

The only requirements for a pair of genomic loci to be used in the present methodology are that each should contain at least one recognition sequence for the methylation sensitive/dependent enzyme (e.g. GCGC in the case of HhaI), and that the methylation ratio should not be uniform across all tissues/cell types.

There are no other requirements for the loci. Specifically, loci do not need to be positioned on any specific chromosome or genomic position, they do not need to be of any specific length, do not necessarily need to be single-copy in the genome, etc.

In order to find recognition sequences for specific endonucleases, a person ordinarily skilled in the art can download any desired genome, and find the locations of any specific endonuclease, which are the locations of the substring of the recognition sequence (e.g. GCGC for HhaI) in the entire string of the genome.

In order to identify candidate pairs of genomic loci whose methylation ratios is not expected to be uniform in different tissues/cell types, and therefore “informative”, a person ordinarily skilled in the art can randomly choose genomic loci and empirically test their usefulness for the assay, or search published data regarding differential methylation of specific genomic regions in different tissues/cell types. See Eckhardt et al, “DNA methylation profiling of human chromosomes 6, 20 and 22” (2006), Nature Genetics 38, 1378-1385 and Straussman et el., “Developmental programming of CpG island methylation profiles in the human genome” (2009), Nature Structural and Molecular Biology 16, 564-571.

There is published data regarding methylation levels in various genomic regions. However, methylation levels per se are meaningless in the context of the assay described here, and there is no published data regarding methylation ratios. Methylation ratios can theoretically be deduced from data regarding methylation levels, however, in reality, in the context of the present assay, this is not feasible because: (1) published methylation levels are in qualitative rather than quantitative (i.e. methylated vs. unmethylated), and for purposes of ratios a numerical value is required; (2) methylation levels between tissues relates to methylation of regions (containing several CGs) rather than specific CGs. For example, in

Straussman et el., island #2, which contains many CGs, is reported to be more methylated in blood than in semen. However this does not mean that any specific CG within that island is more methylated in blood vs. semen, and therefore for any specific CG, the methylation ratio must be checked empirically. (3) existing data is either on a small set of samples or from pooled DNA, and in either case this is insufficient for drawing statistical conclusions on the entire human population. Methylation ratios should be obtained from a number of individuals large enough for reaching statistical significance.

Although the chosen genomic loci can be of any length, it may be advantageous to use relatively short amplicons (less than ˜100bp), since shorter amplicons are more likely to be intact in degraded DNA. In addition, if the assay is intended for use together with DNA profiling, such short amplicons can be useful since their size does not overlap with the size of the fragments commonly used for DNA profiling.

(3) Methylation-Sensitive Restriction Endonucleases

A second consideration of the present methodology is the selection of loci that are or are not cut or digested by a methylation-sensitive and/or methylation-dependent restriction endonuclease. The endonuclease is selected if, for instance, it is unable to cut the DNA strand if its recognition sequence in that locus is methylated. Thus, in the context of locus (1), which is methylated, and locus (2), which is not methylated, an endonuclease like HhaI or HpaII will not digest locus (1) but will digest locus (2). Accordingly, the selection of loci for amplification in the methylation ratio assay may also take into account the presence of methylation-sensitive restriction endonuclease recognition sequences within each locus.

In light of the foregoing, therefore, exemplary characteristics of a suitable pair of loci includes (A) their comparative methylation ratios in different tissue/cell types, and (B) that both loci contain at least one recognition sequence recognized by the same methylation-sensitive restriction endonuclease. In another embodiment, each locus further comprises a short tandem repeat sequence (STR).

Forward and reverse primers can then be designed to anneal to a region of DNA that flanks the recognition sequence of the loci.

Accordingly, in the case of a methylation-sensitive enzyme, if a locus is methylated it will (A) not be digested but (B) it will be amplified. Conversely, if a locus is unmethylated, it will (A) be digested but (B) not amplified. In the case of a methylation-dependent enzyme, the situation is vice versa.

(4) Creation of Reference Distributions

Reference distributions are distributions of methylation ratios obtained from samples of DNA of known sources. For example, a reference distribution for saliva for SEQ26/SEQ31 may consist of 50 methylation ratios of SEQ26/SEQ31 observed and calculated from saliva samples obtained from 50 different individuals.

Thus, to devise reference ratios for different tissues/cell types, the person of ordinary skill in the art can, for example, (1) identify a pair of loci that each contain a recognition sequence for the endonuclease (either methylation-sensitive or methylation-dependent) and which are known to be non-uniform methylation ratios across the different tissues/cell types; (2) digest a sample of DNA from a known tissue/cell type; (3) perform a PCR amplification reaction with PCR primers that are designed to amplify the first and second loci; and (4) determine the intensity of the amplification signals.

The methylation ratio is then calculated by dividing the intensity of the first locus amplification product by the second locus amplification product, or vice versa. If the amplification is performed by fluorescence PCR, then the intensity signal of each amplification product can be readily measured and reported in terms of its relative fluorescent units (rfu). In such a case, the methylation ratio can be obtained by dividing the numerical value of the rfu of the first locus amplification product by the rfu of the second locus amplification product to yield a single number that reflects the methylation ratio between the two known and selected loci from the reference DNA sample. The measurement of fluorescence signals can be performed automatically and the calculation of intensity signal ratios performed by computer software. In order to avoid the problem of division by 0, in case the signal of the denominator is 0, it may arbitrarily be assigned a small positive value.

The foregoing is an example of how the person of skill in the art may systematically determine methylation ratios between two loci selected from DNA of a known tissue/cell type. In so doing, the ordinarily skilled person can create a library of known ratios between various known pairs of genomic loci.

(5) Determining the Tissue/Cell Type Source of DNA

The ordinarily skilled person can determine the most likely source tissue/cell type from the list of methylation ratios, for example, as follows:
1. For each observed methylation ratio, calculate probability scores (between 0-1), one for each tissue/cell type. One way to calculate the probability score for a specific tissue is as follows: one minus two times the absolute difference between 0.5 and the value of the cumulative distribution function of the corresponding reference distribution (of that tissue/cell type) at the observed methylation ratio. This measures how close the observed methylation ratio is to the mean of the specific reference distribution.
2. For each tissue/cell type, calculate a Combined Probability Score (CPS) based on all probability scores of that tissue/cell type as follows: CPS=n-th root of the product of all probability scores, where n is the number of probability scores
3. For each tissue/cell type, calculate a Likelihood Score (LS) as follows:

LS(tissue)=CPS(tissue)/[sum of CPSs of all tissues]
4. The most likely tissue is the tissue with the highest likelihood score.

(6) Capillary Electrophoresis

The rapidity of the analysis is evident in consideration of the use of, for instance, capillary electrophoresis to separate numerous amplification products produced from the amplification of multiple pairs of target loci. As described above the present methylation ratio assay can be performed on multiple loci, and in each case a methylation ratio is calculated for each pair of loci separately. For example, if four loci (A,B,C,D) are co-amplified in the reaction, six different methylation ratios can be calculated, i.e.: A/B, A/C, A/D, B/C, B/D, C/D.

Accordingly, if “n” loci are co-amplified, then (n²−n)/2 different ratios can be calculated. Therefore, the amount of information that is provided by the present methylation assay rises exponentially with the number of analyzed loci. Capillary electrophoresis, as opposed to real-time PCR amplification methods, can distinguish between a large number of loci in a single run. For example, for DNA profiling, 17 genomic loci are routinely co-amplified from a particular DNA sample, and analyzed together. As a consequence, the performance of the present methylation ratio assay on all 17 loci yields 136 independent methylation ratios. Real-time PCR cannot simultaneously distinguish in a single reaction those numbers of discrete amplification products necessary to produce 136 ratios. About four loci can by distinguished by real time PCR, which corresponds to the calculation of only six ratios.

By contrast, capillary electrophoresis can readily separate out amplification products from all paired permutations of 17 loci and can therefore readily produce data to simultaneously calculate all 136 methylation ratios in a single reaction. Theoretically, hundreds of loci can be run together and separated in a single capillary electrophoresis run.

(7) Loci, Primers, and Commercially Available Profiling Kits

Any pair of loci can be used according to the present disclosure to calculate methylation ratios. As discussed elsewhere herein exemplary characteristics of a suitable pair of loci includes (A) they exhibit non uniform methylation ratios in different tissues, (B) that both loci contain at least one recognition sequence recognized by the same methylation-sensitive and/or methylation dependent restriction endonuclease, and, optionally, that (C) each locus contains a short tandem repeat (STR) sequence.

One collection of loci that is used for DNA profiling and which can be used in the present methods, is the U.S. Federal Bureau of Investigation's (FBI) Combined DNA Index System (CODIS). See www.fbi.gov/hq/lab/html/codis1.htm, which is incorporated herein by reference. The CODIS is a collection of thirteen loci identified from the human genome that contain short (or simple) tandem repeat (STR) core sequences. An STR may comprise dimeric, trimeric, tetrameric, pentameric and hexameric tandem repeats of nucleotides. See U.S. Pat. No. 5,843,647 (Simple Tandem Repeats).

The CODIS loci are known as D16S539 (SEQ ID NO. 1), D7S820 (SEQ ID NO. 2), D13S317 (SEQ ID NO. 3), D5S818 (SEQ ID NO. 4), CSF1PO (SEQ ID NO. 5), TPOX (SEQ ID NO. 6), TH01 (SEQ ID NO. 7), vWA (SEQ ID NO. 8), FGA (SEQ ID NO. 9), D21S11 (SEQ ID NO. 10), D8S1179 (SEQ ID NO. 11), D18S51 (SEQ ID NO. 12), and D3S1358 (SEQ ID NO. 13). SEQ ID NOs 1-13 are provided herein.

Other loci that are not included in the CODIS collection but which can be used according to the present disclosure include but are not limited to Penta D (SEQ ID NO. 14), Penta E (SEQ ID NO. 15), and Amelogenin (SEQ ID NOs. 16 and 17); and D2S1338 (SEQ ID NO. 18), D19S433 (SEQ ID NO. 19), ACTBP2SE33 (SEQ ID NO. 20), D10S1248 (SEQ ID NO. 21), D1S1656 (SEQ ID NO. 22), D22S1045 (SEQ ID NO. 23), D2S441 (SEQ ID NO. 24), and D12S391 (SEQ ID NO. 25).

Commercially available kits that are sold for DNA profiling analyses provide PCR amplification primers that are designed to amplify all CODIS and some non-CODIS loci. Promega Corporation's PowerPlex® 16 DNA profiling series is an example of a commercially available collection of primers for amplifying sixteen loci identified as Penta E, D18S51, D21S11, TH01, D3S1358, FGA, TPOX, D8S1179, vWA, Amelogenin, Penta D, CSF1PO, D16S539, D7S820, D13S317 and D5S818. See www.promega.com/applications/hmnid/productprofiles/pp16/ which is incorporated herein by reference. The PowerPlex® 16 kit is particularly useful because it has been approved for forensic DNA profiling use by the European police network, INTERPOL, the European Network of Forensic Science Institutes (ENFSI), GITAD (Grupo Iberoamericano de Trabajo en Análisis de DNA) and the United States Federal Bureau of Investigation (FBI).

As explained in more detail below, the present disclosure encompasses the use of a kit, such as the PowerPlex® 16 profiling kit, in conjunction with one or more primers for amplifying additional loci that are not contained within the kit. As a non-limiting example, these additional locus may be selected because they are known to be differentially methylated in various tissues/cell types. Examples of such additional loci include but are not limited to SEQ ID NOs. 26-31. Thus, in accordance with the methylation ratio assay described herein, the ordinarily skilled person will expect a methylation-sensitive enzyme, such as HhaI, to properly bind and cut the unmethylated HhaI restriction site in these loci.

In another aspect of the present disclosure, prior knowledge of the sequence or methylation characteristics of a particular locus or pair of loci is not a prerequisite to performing an assay described herein. That is, an assay of the present disclosure encompasses the random selection of loci and the subsequent comparison of paired random loci amplified from a restriction-digested DNA sample to yield ratios that can be compared against control or threshold ratio values indicative of, for instance, the tissue/cell type source of the DNA sample.

D. Combination of CODIS, Kits, and Methylation Assay

Accordingly, the combination of a CODIS or PowerPlex® 16 kit and the additional loci enables to simultaneously profile a DNA sample and determine the tissue/cell type source of the sample. For instance, the present methodology contemplates digesting a DNA sample with HhaI, and amplifying the DNA with the PowerPlex® 16's kit, to which primers for loci from SEQ ID NOs: 26-31 are added.

Analysis of loci SEQ ID NOs: 26-31, as described above, will yield the determination of the tissue/cell type source of the DNA sample, whereas the analysis of the profiling loci (e.g. PowerPlex16 loci) will yield the determination of the DNA profile.

Thus, a powerful aspect of the present inventive technology is its ability to transform and expand the usefulness of existing commercial DNA profiling kits to do more than profile a particular subject's DNA. The combination of the inventive assays disclosed herein, such as the methylation ratio assay, with, for instance, the PowerPlex® 16 kit, enables the user to test the profiled DNA and determine the tissue/cell types source of the DNA.

(1) DNA Profiling Kits

Other examples of DNA profiling kits whose usefulness can be enhanced to determine also the tissue/cell type source of the DNA sample include but are not limited to SGM, SGM+, AmpFlSTR Identifiler, AmpFlSTR Profiler, AmpFlSTR ProfilerPlus, AmpFlSTR ProfilerPlusID, AmpFlSTR SEfiler, AmpFlSTR SEfiler Plus, AmpFlSTR Cofiler, AmpFlSTR Identifiler Direct, AmpFlSTR Identifiler Plus, AmpFlSTR NGM, AmpFlSTR Y-filer, AmpFlSTR Minifiler, PowerPlex1.1, PowerPlex2.1, PowerPlex16, PowerPlexES, PowerPlexESX16, PowerPlexESI16, PowerPlexESX17, and PowerPlexESI17.

(2) Sequences

The sequences provided herein for the various CODIS, PowerPlex® 16, and other loci commonly used for profiling, i.e., SEQ ID NOs. 1-25, have been analyzed herein to determine (1) the position of every cytosine-guanine (CG) dinucleotide, (2) the methylation-sensitive and methylation-dependent restriction enzyme profile for that particular locus. The sequence listing included within the text of this application therefore provides guidance to the ordinarily skilled person in the identification of particular methylation-sensitive and methylation-dependent restriction endonucleases that can be used in accordance with ratio-generating assay methods.

The sequence information provided herein also permits the ordinarily skilled artisan to design forward and reverse amplification primers that anneal to regions of a selected locus that flank the CG and restriction site. Thus, the present disclosure is not limited to the amplification of, for instance, CODIS loci, using only those commercially available primers, although the use and availability of commercially available primers can be a more convenient and cost-effective option for performing the present authentication assays.

(3) Correction for “Ski-Slope” Effect

A common problem with some electropherogram trace outputs is an artifact known as a “ski slope.” A “ski slope” is the name given to an artifact that is sometimes observed in electropherograms and which manifests in an inverse relationship between amplicon size and signal intensity. In such electropherograms, the signals resemble a “ski-slope” tail, the trace of which runs down and to the right. This artifact can be caused by several factors, for example by sample overload (too much DNA template in PCR) or from degraded DNA.

The present assays correct for this artifact in the calculation of methylation ratios by performing a normalization step. Typically, the normalization process entails (1) obtaining a linear fit for the sample from a subset of loci; (2) normalizing all peak values to the linear fit obtained in (1); and (3) calculating methylation ratios based on normalized peak values. Specific loci used for calculation of linear fit in PowerPlex® 16 were determined herein as D3S1358, TH01, D21S11, Penta_E.

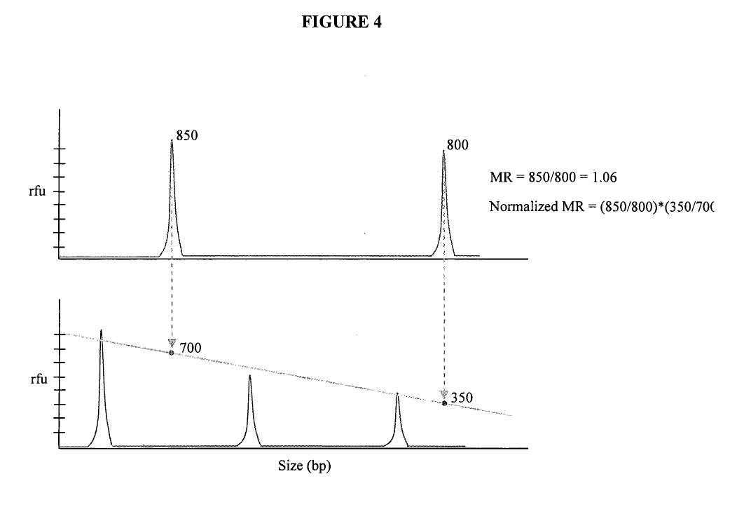

A criterion for deciding which subset of loci are useful for calculating the linear fit is whether the loci are uninformative in relation with the tested character. Specifically, they should not contain the recognition sequence of the restriction enzyme used in the assay, or else should have similar methylation ratios in all relevant tissues. For example, for the PowerPlex16 kit it was found herein that this subset consists of the loci D3S1358, TH01, D21S11, Penta_E. Once the subset of loci is determined, the linear fit can be calculated, for example, by performing the least squares method on the relative fluorescent unit (rfu) signals of this particular subset of loci. Subsequent normalizing of a peak value can be achieved, for example, by dividing the rfu of the peak by the value of the linear fit at the same X-axis coordinate (size in bp). See, e.g., FIG. 4.

(4) Algorithm and Software

In one embodiment, calculation of methylation ratios is performed based on analysis of the intensities of signals of amplification products of fluorescent PCR that are run on a capillary electrophoresis machine. The output of the capillary electrophoresis machine is a binary computer file (for example, an FSA file in the case of capillary electrophoresis machines of Applied Biosystems). This file includes information regarding the capillary electrophoresis run, including the channel data, which is the relative fluorescent units (rfus) of each fluorophore as a function of each sampling time point (called datapoint).

The present disclosure also describes a software program that accepts as input a file that is the output a capillary electrophoresis machine run, and calculates the fluorescence intensities of a set of loci whose amplification products were run on the capillary electrophoresis machine. Based on these intensities, the software calculates methylation ratios, based on a set of predefined pairs of loci for which the ratios are defined to be calculated. Finally, the software outputs the tissues/cell type that is most likely the source of the DNA sample

Following is a scheme of this analysis performed by the software program:

1. Read the channel data of each fluorophore. This requires knowledge of the specific format in which the channel data is encoded in the capillary electrophoresis output file. In the case of FSA files, the format is explained in detail in a document written by Applied Biosystems (which is available online at www.appliedbiosystems.com/support/software_community/ABIF_File_Format.pdf), enabling a person skilled in the art to write a computer program to obtain the channel data (and other information regarding the run) from this file.

2. Perform baseline reduction for the channel data of each fluorophore. Each fluorophore has a basal fluorescent intensity level, meaning that even when no amplification products labeled by that fluorophore are detected at a certain datapoint, the rfu level of that fluorophore will be non-zero at that datapoint. In order to perform correct analysis, the baseline level of each fluorophore needs to be removed by reducing the baseline level from the rfu level at all time-points. The baseline level of each fluorophore can be obtained, for example, by averaging the rfu level of that fluorophore in parts of the run in which there were no amplification products for that fluorophore. Because normally most of the capillary electrophoresis run is devoid of amplification products, finding such regions is not a difficult task for a person skilled in the art.

3. Remove spectral overlap between fluorophores. The fluorescent dyes used in capillary electrophoresis have distinct maximum emission lengths, but nevertheless they have overlapping emission spectra. This means that certain dyes “pull-up” other dyes, creating artifact rfu levels in the other dyes. In order to perform correct analysis, these pull-ups need to be removed. This can be performed by knowing the n*n matrix of pull-ups (where n is the number of dyes), in which the (i,j) element is the fraction by which dye i pulls-up dye j. This matrix can be obtained by running on the dye set the spectral calibration procedure on the capillary electrophoresis machine

4. Detect peaks. Certain parts of the channel data are peaks signals, each corresponding to a specific amplification product. An amplification product can correspond for example to an allele of a profiling locus, a control locus, or a peak in the standard curve. Peaks in capillary electrophoresis data have distinct patterns that enable to detect them, and a person skilled in the art knows this distinct pattern. Based on this, an algorithm for peak detection can be designed. One example for such a peak detection algorithm is as follows: detect all local maxima (i.e. datapoints at which the rfu level is greater than the rfu level of both two neighboring datapoints) and define each such local maxima as peaks with a height equal to the rfu level at the local maxima point. Because not all local maxima correspond to peaks, excessive peaks need to be removed. One way to remove excessive peaks is, for example, based on the idea that a peak must have the highest rfu level in its close vicinity (within its X neighboring datapoints). Based on this, excessive peaks are removed by going over all peaks, and removing any peak that is close (within X datapoints, where X is some pre-defined parameter) to another higher peak.

5. Assign sizes in basepairs to peaks. Channel data for each fluorophore is obtained as a set of rfu levels as a function of datapoints. Datapoints correlate to basepairs, but the exact function correlating between the two needs to be determined. For this purpose, a standard curve—a set of amplification products with known lengths in basepairs—is run together with the sample amplification products (whose lengths are unknown). Based on the standard curve peaks, a fit correlating datapoints and basepairs is obtained. This fit can be obtained using one of several methods known in the art, for example using the Least Squares method. Once a fit is obtained, all detect peaks are assigned their sizes in basepairs.

6. Obtain the signal intensities of the loci used for analysis. The expected size of each analyzed locus is known a priori. Loci can be polymorphic (e.g. as used for profiling), and in this case their expected size is within a certain range based on the set of possible alleles of that locus. Other loci are non-polymorphic (e.g. control loci), in which case their expected size is within a smaller range. The signal intensity of each locus is the sum of rfus of non-artifact peaks within the range of the locus (e.g. the two peaks corresponding to the two alleles of a profiling locus).

7. Obtain the methylation ratios. Once signal intensities are calculated for all loci, a methylation ratio between a pair of loci is the division of the signal intensity of the first locus in the pair by the signal intensity of the second locus in the pair.

8. Calculate probability and combined probability scores. Probability scores can be calculated based by comparing methylation ratios to reference distributions of methylation ratios obtained from different tissues/cell types. Combined Probability Scores (CPS) of each tissue/cell type can then be calculated from the single probability scores, for example by calculating the n-th root of the product of the single probability scores (where n is the number of methylation ratios).

9. Calculate likelihood scores. For each tissue/cell type, calculate a Likelihood Score (LS), that represents the likelihood that the DNA sample originated from that tissue/cell type. Likelihood scores for each tissue/cell type can be calculated for example as follows:

LS(tissue)=CPS(tissue)/[sum of CPSs of all tissues]

10. Output the tissue/cell type with the highest LS.

### (5) Determining the Source of a Mixed DNA Sample

In some cases, the DNA sample is not of pure source, but rather is a mixture of two or more source (e.g. 50% blood and 50% semen). The present invention can also determine the makeup of source of such a sample by performing the following analysis:

(a) digesting the DNA sample with a methylation-sensitive and/or methylation-dependent restriction endonuclease;

(b) amplifying the digested DNA with at least a first and a second restriction locus, thereby generating an amplification product for each restriction locus;

(c) determining the intensity of the signal of each amplification product;

(d) calculating at least one methylation ratio between the intensity of the signals corresponding to the two restriction loci;

(e) comparing the methylation ratio calculated in step (d) to a set of reference methylation ratios obtained from DNA of known tissues and/or cell types;

(f) determining the likelihood of each tissue and/or cell type contributing to the source of DNA; and (g) determining the composition of the source DNA based on the likelihoods obtained in step (f)

### EXAMPLES

### Example 1

### Tissue Identifier Assay Based on Genomic Loci

In this example a tissue identifier assay was developed that is capable of distinguishing between DNA samples obtained from blood, semen, and skin epidermis. The assay is based on the analysis of six specific genomic loci, each set forth in SEQ ID NOs:. 26-31. Each locus is a fragment sized 70-105 bp containing a HhaI restriction site (GCGC). The enzyme HhaI cleaves its recognition sequence only if it is unmethylated, therefore the assay is based on differences in methylation in the recognition sequences only. The six genomic loci each contain additional CGs whose methylation status is of no consequence to the assay—only the methylation of the recognition sequence is relevant. The sequences of the six genomic loci are:

SEQ ID NO: 26 (Chr. 3):

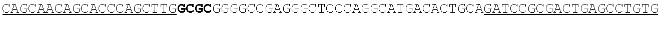

SEQ ID NO: 27 (Chr. 10):

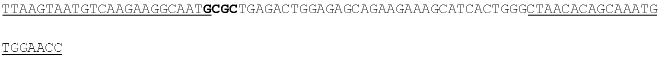

SEQ ID NO: 28 (Chr. 1):

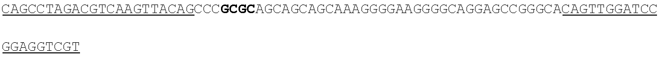

SEQ ID NO: 29 (Chr. 5):

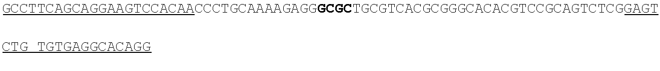

SEQ ID NO: 30 (Chr. 3):

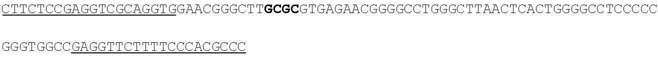

SEQ ID NO: 31 (Chr. 22):

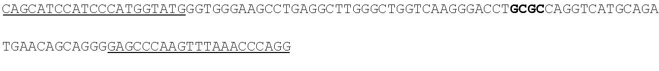

Primer sequences are underlined and shaded, HhaI recognition sequences are bolded.

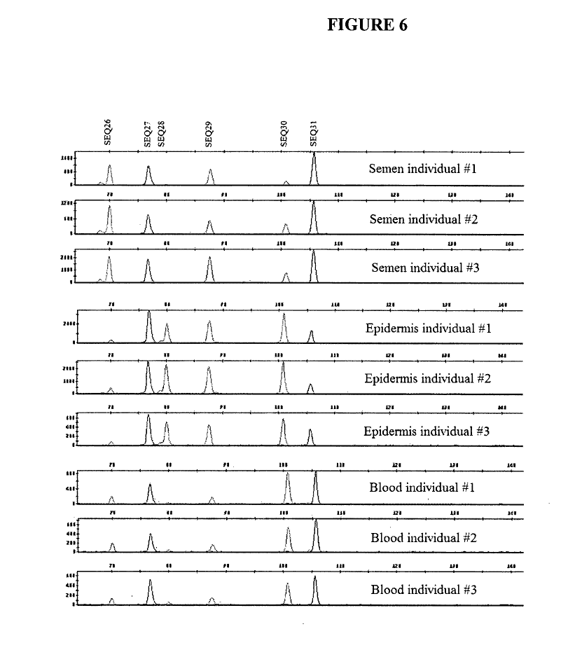

The assay was performed on DNA samples extracted from semen, epidermis and blood of three different individuals (total of nine samples). One nanogram of each DNA sample was mixed with HhaI, Taq Polymerase, forward (fluorescently-labeled) and reverse primers for the six loci SEQ ID NOs: 26-31, dNTPs, and reaction buffer in a single microcentrifuge tube. The tube was then placed in a thermocycler and subject to a single program that contains an initial digestion step (37° C.), followed by PCR amplification of digestion products. Following the restriction-amplification reaction, an aliquot of the products was run on a capillary electrophoresis machine. FIG. 6 shows the electropherograms of capillary electrophoresis of the nine samples. In each electropherograms, there are six peaks, each corresponding to one locus. The data from the electropherograms of the nine samples was then analyzed as follows: for each sample, the intensity of the signal (rfu) in each locus was quantified, and methylation ratios (e.g. rfu of locus 1 divided by rfu of locus 2) were calculated for all 15 loci pair combinations (e.g. SEQ ID NO: 26/SEQ ID NO: 28).

Table 1 shows values of two of the fifteen such methylation ratios (SEQ ID NO: 29/SEQ ID NO: 30 and SEQ ID NO: 28/SEQ ID NO: 26) for all samples. For each sample, each methylation ratio was compared to the cumulative distribution functions of its reference distributions in blood, semen and epidermis (obtained empirically from a large set of DNA samples from blood, semen, and epidermis)

TABLE 1
Methylation ratios for two pairs of loci in the nine analyzed samples.

| | SEQ29/SEQ30 | SEQ28/SEQ26 |
|---|---|---|
| Semen individual #1 | 4.01 | 0.04 |
| Semen individual #2 | 1.27 | 0.02 |
| Semen individual #3 | 2.54 | 0 |
| Epidermis individual #1 | 0.76 | 6.68 |
| Epidermis individual #2 | 0.81 | 5.38 |
| Epidermis individual #3 | 0.76 | 6.41 |
| Blood individual #1 | 0.21 | 0.18 |
| Blood individual #2 | 0.30 | 0.25 |
| Blood individual #3 | 0.33 | 0.42 |

Table 2 shows means and standard deviations of reference distributions for two methylation ratios (obtained empirically from a large set of DNA samples from blood, semen, and epidermis).

TABLE 2
Reference methylation ratio values for two pairs of loci (mean ± std)

| | SEQ29/SEQ30 | SEQ28/SEQ26 |
|---|---|---|
| Semen | 2.8 ± 1.1 | 0.02 ± 0.04 |
| Epidermis | 0.78 ± 0.06 | 6.21 ± 0.7 |
| Blood | 0.29 ± 0.04 | 0.28 ± 0.08 |

For each tissue/cell type, each comparison between the observed methylation ratio and its corresponding value in the cumulative distribution function yielded a Probability Score, calculated as follows:

PS(Blood, SEQ26/28)=1−[2*abs(f(OMR)−0.5)], where f is the cumulative distribution function of the reference distribution of SEQ26/28 in blood, and OMR is the observed methylation ratio of SEQ26/28 in the sample.

PS(Semen, SEQ26/28) and PS(Epidermis, SEQ26/28) were calculated in a similar manner.

Next, Combined Probability Scores (CPS) were calculated for each tissue type based on all methylation ratios as follows:

CPS(Blood)=nth root of [LS(Blood, methylation ratio #1)*LS(Blood, methylation ratio #2) * . . . * LS(Blood, methylation ratio #n)], where n is the number of methylation ratios

CPS(Semen) and CPS(Epidermis) were calculated in a similar manner.

Finally, Likelihood Scores (LS) were calculated from the combined probability scores as follows:

LS(Blood)=CPS(Blood)/[CPS(Blood)+CPS(Semen)+CPS(Epidermis)]

LS(Semen) and LS(Epidermis) were calculated in a similar manner.

The likelihood score of each tissue/cell type represents the likelihood that the DNA sample originated from that specific tissue/cell type.

Table 3 shows likelihood scores for the three tissues based on all methylation ratios for all 9 DNA samples.

TABLE 3
Likelihood scores based on all methylation ratios

| | Combined likelihood scores | | |
|---|---|---|---|
| | based on all pairs of loci | | |
| | Semen | Epidermis | Blood |
| Semen individual #1 | >0.9999 | <0.0001 | <0.0001 |
| Semen individual #2 | >0.9998 | <0.0001 | <0.0001 |
| Semen individual #3 | >0.9999 | <0.0001 | <0.0001 |
| Epidermis individual #1 | <0.0001 | >0.9999 | <0.0001 |
| Epidermis individual #2 | <0.0001 | >0.9998 | <0.0001 |
| Epidermis individual #3 | <0.0001 | >0.9998 | <0.0001 |
| Blood individual #1 | <0.0001 | <0.0001 | >0.9999 |
| Blood individual #2 | <0.0001 | <0.0001 | >0.9999 |
| Blood individual #3 | <0.0001 | <0.0001 | >0.9999 |

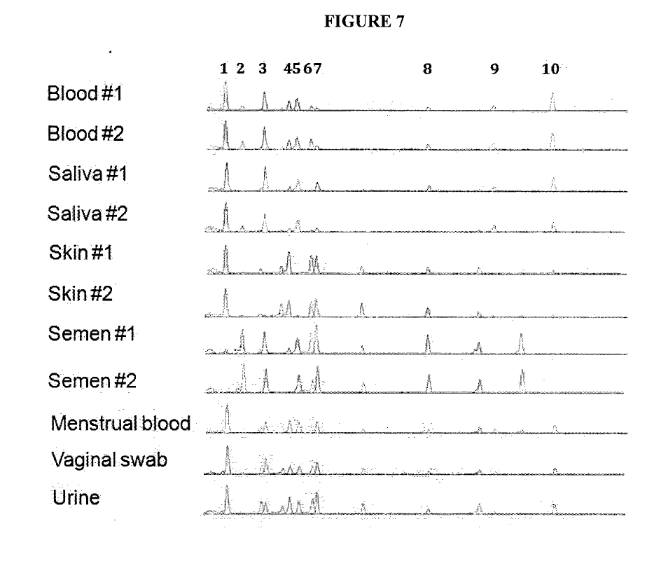

Similarly, and as shown in FIG. 7, a tissue identification assay was performed using a 10-loci multiplex on 11 different DNA samples from blood, saliva, skin, semen, menstrual blood, vaginal swab, and urine. Analysis was based on 45 methylation ratios (e.g. locus1/locus 2, locus1/ locus 3, etc.). Differential methylation across blood, saliva, skin, semen, menstrual blood, vaginal tissue, and urine is evidenced by the different intensities of the analyzed loci. The assay correctly identified the source tissue of all samples. For example, and as shown in FIG. 7, DNA derived from menstrual blood can be differentiated from DNA derived from saliva.

SEQUENCES, METHYLATION PROFILE, CG SITES, & RESTRICTION
SITES

Sequence 1: D16S539
Amplicon length = 889 bps

| | |
|---|---|
| 1 | CTCTTCTCAT TCCACAAGCT CTCCCCAAAA GACCCCATTC CTCCCCACCT TCAACCATCT |
| 61 | CTGGCAGGGA GGAGGGGGAA CTGAGAGGCT ACTTTCTGAC CCAGGACCCT AAGCCTGTGT |
| 121 | ACGGAGAGAG CATGAGCTGG GTGAGCTGCT TGCCAAGGAG TGGCATCTGC CCTCATCAGT |
| 181 | GGACACAAAA AGCCCCAGGG GTTAAGTGGC CATGGCTGCC CTCATGGCTG CACCGGGAGG |
| 241 | ATGACTGTGT TCCCACTCTC AGTCCTGCCG AGGTGCCTGA CAGCCCTGCA CCCAGGAGCT |
| 301 | GGGGGGTCTA AGAGCTTGTA AAAAGTGTAC AAGTGCCAGA TGCTCGTTGT GCACAAATCT |
| 361 | AAATGCAGAA AAGCACTGAA AGAAGAATCC AGAAAACCAC AGTTCCCATT TTTATATGGG |
| 421 | AGCAAACAAA GGCAGATCCC AAGCTCTTCC TCTTCCCTAG ATCAATACAG ACAGACAGAC |
| 481 | AGGTGGATAG ATAGATAGAT AGATAGATAG ATAGATAGAT AGATAGATAT CATTGAAAGA |
| 541 | CAAAACAGAG ATGGATGATA GATACATGCT TACAGATGCA CACACAAACG CTAAATGGTA |
| 601 | TAAAAATGGA ATCACTCTGT AGGCTGTTTT ACCACCTACT TTACTAAATT AATGAGTTAT |
| 661 | TGAGTATAAT TTAATTTTAT ATACTAATTT GAAACTGTGT CATTAGGTTT TTAAGTCTAT |
| 721 | GGCATCACTT TCGCTTGTAT TTTTCTATTG ATTTCTTTTC TTTTCTTTTC TTTTTTGAGA |
| 781 | CAGAGTCTCA CTCTCACCCA GGCTGGAGTA CCGTGGCACG ATCTTGGCTC ATTGCAACCA |
| 841 | CCACCTCCCG GGCTCAAGTG ATTATCCTGC CTCAGCCTCC CAAATAGCT |

CG locations, methylation status and restricting enzymes:

| | |
|---|---|
| 122: | BslI Hpy166ii RsaI McrBC (half site) |
| 234: | BssKI HpaII Nt.CviPII ScrFI |
| 269: | Nt.CviPII |
| 345: | MwoI |
| 589: | McrBC (half site) |
| 732: | |
| 812: | Nt.CviPII |
| 819: | McrBC (half site) |
| 849: | AvaI BslI BssKI HpaII Nt.CviPII ScrFI SmaI TspMI |

-----------------------------------------------------------------------------------
Sequence 2: D7S820
Amplicon length = 843 bps

| | |
|---|---|
| 1 | ATATGCTAAC TGGATGTGAA CAATTGTGTT CTAATGAGCT TAATATGAGT TTCATAATTT |
| 61 | GTGCATTTTG CTGTTAAAAA GCCAGAAAAC AAAACAAAAC AAAATACTGA AACCAGTGTG |
| 121 | AACAAGAGTT ACACGATGGA AGGCATCAGT TTTCACACCA GAAGGAATAA AAACAGGCAA |
| 181 | AAATACCATA AGTTGATCCT CAAAATATGA TTGATTTTAA GCCTTATGAG ATAATTGTGA |
| 241 | GGTCTTAAAA TCTGAGGTAT CAAAAACTCA GAGGGAATAT ATATTCTTAA GAATTATAAC |
| 301 | GATTCCACAT TTATCCTCAT TGACAGAATT GCACCAAATA TTGGTAATTA AATGTTTACT |
| 361 | ATAGACTATT TAGTGAGATT AAAAAAAACT ATCAATCTGT CTATCTATCT ATCTATCTAT |
| 421 | CTATCTATCT ATCTATCTAT CTATCTATCT ATCGTTAGTT CGTTCTAAAC TATGACAAGT |
| 481 | GTTCTATCAT ACCCTTTATA TATATTAACC TTAAAATAAC TCCATAGTCA GCCTGACCAA |
| 541 | CATGGTGAAA CCCCGTCTCT AAAAAAAATA CAAAAATTAG CTGGATGCAG TAGCACATGC |
| 601 | CTGTAGTCCC AGCTACTCAG GAGGCTGGGG CAGGAGAACC ACTTGACCCA AGAAGCGGAG |
| 661 | GTTGCAGTGA GCCGAGATCG CACCACTGCA CTCCAGCCTG GGTGACAGAG TGAGACTCCA |
| 721 | TCTCAAGATA AAGAAATAAA TAAAAACAAA CAAACAAAAA AATTCCATAG GGGGTCAGGT |
| 781 | GCGGTGGCTC ATGCCTGTAA TCCCAGCACT TTGGGAGGCC GAAGCAGGTG GATCACTTGA |
| 841 | GGT |

CG locations, methylation status and restricting enzymes:

| | |
|---|---|
| 134: | McrBC (half site) |
| 300: | McrBC (half site) |
| 453: | |
| 461: | |
| 554: | BsmBI Nt.CviPII |
| 656: | BslI MwoI McrBC (half site) |
| 673: | MwoI Nt.CviPII |
| 679: | BfuCI DpnI MwoI Sau3AI |
| 782: | McrBC (half site) |
| 820: | Nt.CviPII |

-----------------------------------------------------------------------------------
Sequence 3: D135317
Amplicon length = 792 bps

| | |
|---|---|
| 1 | AATATGAATT CAATGTATAC AGAGAGAGAG AGAGAGAGAG AGAGAGAGAG AGACTTCTAC |
| 61 | AGAGCTCTAA GCATAATTGT GTAACTCCAA GCTCACAGTG CCTAAGACCA GTACCAGGCT |
| 121 | GACTCATTGG AAAGCTGCCA TAGTAAGACT CTTCTGTTCA CTGCATTATT TATTGATGTA |
| 181 | TTGCAAGCAC TTAGTTACAT TTCTAGCATA TAACACATGA TCAATAAATA TTTTGACATG |
| 241 | AACAAATGGT AATTCTGCCT ACAGCCAATG TGAATATTGG GATGGGTTGC TGGACATGGT |
| 301 | ATCACAGAAG TCTGGGATGT GGAGGAGAGT TCATTTCTTT AGTGGGCATC CGTGACTCTC |
| 361 | TGGACTCTGA CCCATCTAAC GCCTATCTGT ATTTACAAAT ACATTATCTA TCTATCTATC |
| 421 | TATCTATCTA TCTATCTATC TATCTATCAA TCAATCATCT ATCTATCTTT CTGTCTGTCT |
| 481 | TTTTGGGCTG CCTATGGCTC AACCCAAGTT GAAGGAGGAG ATTTGACCAA CAATTCAAGC |
| 541 | TCTCTGAATA TGTTTTGAAA ATAATGTATA TTAATGAATG TACAAATTTC CCCACTTGTA |
| 601 | CTTTCAGACT GTTATCTGTG AGTTAAAACT CCTCCACTCT TTTTCCTACC CAAATAATAG |
| 661 | CATACTTTTT TCTGAGTATA TTTTGGGAAG AAGAGTTATT CAGTTATTGT TATATTTTAA |
| 721 | AAAATTCCTT ATACCAAACT CTACTTGATC TAAGGCTATT CATTGAAACT TTCAGCATGC |
| 781 | TTAATAGCAG TC |

CG locations, methylation status and restricting enzymes:

| | |
|---|---|
| 351: | Nt.CviPII |
| 380: | McrBC (half site) |

-----------------------------------------------------------------------------------
Sequence 4: D5S818
Amplicon length = 735 bps

| | |
|---|---|
| 1 | CCCTCTGTGT AGCCTGGCTA TGTGCCACAT TGTGAGGTTC TCTCCCTCTA GCTACTTCTT |
| 61 | CCAGTTTCCT CTTTCAGGAT CCCAATTCCT TTCTCAAAGC ACTGGTGAAT AACTCCAAAT |
| 121 | ACTCCATCAT TTCATTATAC AGAGTAATAT AAGTCTTCTT TTTCTAAACC TCTCCCATCT |
| 181 | GGATAGTGGA CCTCATATTT CAGATGCTAA TAGGCTGTTG AGGTAGTTTC CTAAGCAAAA |
| 241 | AAGTAATTGT CTCTCTCAGA GGAATGCTTT AGTGCTTTTT AGCCAAGTGA TTCCAATCAT |
| 301 | AGCCACAGTT TACAACATTT GTATCTTTAT CTGTATCCTT ATTTATACCT CTATCTATCT |
| 361 | ATCTATCTAT CTATCTATCT ATCTATCTAT CTATCTTCAA AATATTACAT AAGGATACCA |
| 421 | AAGAGGAAAA TCACCCTTGT CACATACTTG CTATTAAAAT ATACTTTTAT TAGTACAGAT |
| 481 | TATCTGGGAC ACCACTTTAA TTAGAAGCTT TAAAAGCATA TGCATGTCTC AGTATTTAAT |
| 541 | TTTAAAATTA TTACATAATT ATATACTCCT TTGAATTAGA AAATTACAAA TGTGGCTATG |
| 601 | TATTATTTTC TCCCATGTAT TTTCAAAATG AGGGGGTAAG CCAGACCCTC TCCCTCTCCC |
| 661 | ATGCCTAATA GCTCAAAGTT AAAGGTAAAG AAACAAGAAA ATATGGTGAA AGTTAACCAG |
| 721 | CTTCACTTCA GAGGA |

CG locations, methylation status and restricting enzymes:
-----------------------------------------------------------------------------------
Sequence 5: CSF1PO
Amplicon length = 949 bps

| | |
|---|---|
| 1 | ATTCAACACA TGAGGCACGG CCAGACCTAA ATGTCTCAGA GCCTGCTCCC ACTCCGATGA |
| 61 | GCTGCTGCCT TGCTTCAGGG TCTGAGTCCA GTGACTGCCA CTGCCTGCAC CCAATCACCA |
| 121 | TAGCCAGAGA CCTGGAGGTC ATCCTTATCT CCTTTCTCTT CCTCATCCCT GCATCTCAGA |
| 181 | CTCTTCCACA CACCACTGGC CATCTTCAGC CCATTCTCCA GCCTCCAGGT TCCCACCCAA |
| 241 | CCCACATGGT GCCAGACTGA GCCTTCTCAG ATACTATCTC CTGGTGCACA CTTGGACAGC |
| 301 | ATTTCCTGTG TCAGACCCTG TTCTAAGTAC TTCCTATCTA TCTATCTATC TATCTATCTA |
| 361 | TCTATCTATC TATCTATCTA TCTATCTAAT CTATCTATCT TCTATCTATG AAGGCAGTTA |
| 421 | CTGTTAATAT CTTCATTTTA CAGGTAGGAA AACTGAGACA CAGGGTGGTT AGCAACCTGC |
| 481 | TAGTCCTTGG CAGACTCAGG TTGGAACACT GCCCTGGAGT GTGTGCTCCT GACCACCACG |
| 541 | AAGTGCCTCC TCTGTACAAT CTGACCCCAT CACTCTCCTC TTTACAATGA CCTCCCAATA |
| 601 | GGTTAAGATG CAGTTATTCT TTCTCACTTT AAGACACCTT TACCTCCGGC TTCTGCCACC |
| 661 | TCCTCTGCTC CCCTGTGGCC ACTCCTCACA CCACTCCACA TCCCAGCTGT TGTCAAGTTC |
| 721 | TTTCAGTGTT CCAAATGATC TATGTTCTCT TTGCCTTTGA GCCTTGCATA TGTTCCTCCC |
| 781 | TCTGCCAGAA GCGCTGTTCC CCTTCCTTTC CCACCCTTCT GCCCGGCCAA CTCACCTTCA |
| 841 | TTCTTCCCAT CCCAGTTTAG AGGCCACCTT CTCGAAGCCT GGGTTGGGGG GACTCTTCAG |
| 901 | TGTTCCCAGG ACACCTTGTG CTTCCCCCAT AATCACTGGG TGATCATTG |

CG locations, methylation status and restricting enzymes:

| | |
|---|---|
| 18: | BceAI EaeI McrBC (half site) |
| 55: | Nt.CviPII |
| 539: | McrBC (half site) |
| 647: | HpaII Nt.CviPII |
| 792: | AfeI HaeII HhaI HinPlI MwoI McrBC (half site) |
| 824: | BssKI EaeI HpaII Nt.CviPII ScrFI |
| 873: | Hpy188iii |

-----------------------------------------------------------------------------------
Sequence 6: TPOX
Amplicon length = 832 bps

| | |
|---|---|
| 1 | CCCAGCACAC ACCTTGCCTC TGGCTGGGAC CCCCTTTGCT GCTGGCCCTG CTCAGGCCCC |
| 61 | ACAGCTTGAT CTCCTCATGT TCCCACTGCT GACTTCCCCA AGCTAACTGT GCCACAGAGT |
| 121 | GGGGGACCCC CTCCCGGCTC TCACAACCCC CACCTTCCTC TGCTTCACTT TTCACCAACT |
| 181 | GAAATATGGC CAAAGGCAAA AACCCATGTT CCCACTGGCC TGTGGGTCCC CCCATAGATC |
| 241 | GTAAGCCCAG GAGGAAGGGC TGTGTTTCAG GGCTGTGATC ACTAGCACCC AGAACCGTCG |
| 301 | ACTGGCACAG AACAGGCACT TAGGGAACCC TCACTGAATG AATGAATGAA TGAATGAATG |
| 361 | AATGAATGTT TGGGCAAATA AACGCTGACA AGGACAGAAG GGCCTAGCGG GAAGGGAACA |
| 421 | GGAGTAAGAC CAGCGCACAG CCCGACTTGT GTTCAGAAGA CCTGGGATTG GACCTGAGGA |
| 481 | GTTCAATTTT GGATGAATCT CTTAATTAAC CTGTGGGGTT CCCAGTTCCT CCCCTGAGCG |
| 541 | CCCAGGACAG TAGAGTCAAC CTCACGTTTG AGCGTTGGGG ACGCAAACAC GAGAGTGCTT |
| 601 | GGTGTGAGCA CACAGGAGGA GTCACGACAG AGCAGTGTAA GAGCCGCCAC GTGGGTCCCA |
| 661 | CACAGGGGGA GTCACGACAC AGCAGTGTAA GAGCCGCCAC GAGGGTCCCA CACAGGGGGA |
| 721 | GTCGCGACAC AGCAGTGTAA GAGCCGCCAC GAGGGTCCCA CACAGGGGGA GTCACGACAC |
| 781 | AGCAGTGTAA GAGCCGCCAC GAGGGTCCCA CACAGGGGGA GTCACGACAC AG |

CG locations, methylation status and restricting enzymes:

| | |
|---|---|
| 135: | BslI BssKI HpaII Nt.CviPII ScrFI |
| 240: | BfuCI DpnI Sau3AI |
| 296: | BslI Hpy99I Nt.CviPII |
| 299: | AccI BslI HincII HincII Hpy166ii Hpy99I SalI SalI-HF |
| 383: | McrBC (half site) |
| 408: | McrBC (half site) |
| 434: | HhaI HinPlI McrBC (half site) |
| 443: | Nt.CviPII |
| 539: | HaeII HhaI HinPlI McrBC (half site) |
| 565: | HpyCH4IV TscI McrBC (half site) |
| 573: | McrBC (half site) |
| 582: | BsmFI HgaI McrBC (half site) |
| 590: | McrBC (half site) |
| 625: | Hpy188iii McrBC (half site) |
| 645: | AciI BslI Fnu4HI Nt.CviPII |
| 650: | BsaAI BslI HpyCH4IV PmlI TscI McrBC (half site) |
| 675: | Hpy188iii McrBC (half site) |
| 695: | AciI BslI Fnu4HI Nt.CviPII |
| 700: | BslI McrBC (half site) |
| 723: | BstUI HinfI Hpy188iii NruI PleI |
| 725: | BstUI Hpy188iii NruI McrBC (half site) |
| 745: | AciI BslI Fnu4HI Nt.CviPII |
| 750: | BslI McrBC (half site) |
| 775: | Hpy188iii McrBC (half site) |
| 795: | AciI BslI Fnu4HI Nt.CviPII |
| 800: | BslI McrBC (half site) |
| 825: | Hpy188iii McrBC (half site) |

-----------------------------------------------------------------------------------
Sequence 7: TH01
Amplicon length = 766 bps

| | |
|---|---|
| 1 | TTACCCTTGG GGTGGGGGTG TAGGATGCAG CTGGGGCTGC AGTTCCAGGC CACGGAGAGC |
| 61 | CTGTGAGGCT GGGCCCCGGG GCGCCCTGGG GAGGGGATGC CTGATGGGGA GCCTGGTGGG |
| 121 | GGAGGGTAGG GGAGGGCGGG GGAGGACGGG GGAGGGCGCC CTGTGTCCCT GAGAAGGTAC |
| 181 | CTGGAAATGA CACTGCTACA ACTCACACCA CATTTCAATC AAGGTCCATA AATAAAAACC |
| 241 | CATTTTAAAT GTGCCAGGGA GCCCAAGGTT CTGAGTGCCC AAGGAGGCAC CGAAGACCCC |
| 301 | TCCTGTGGGC TGAAAAGCTC CCGATTATCC AGCCTGGCCC ACACAGTCCC CTGTACACAG |
| 361 | GGCTTCCGAG TGCAGGTCAC AGGGAACACA GACTCCATGG TGAATGAATG AATGAATGAA |
| 421 | TGAATGAATG AGGGAAATAA GGGAGGAACA GGCCAATGGG AATCACCCCA GAGCCCAGAT |
| 481 | ACCCTTTGAA TTTTGCCCCC TATTTGCCCA GGACCCCCCA CCATGAGCTG CTGCTAGAGC |
| 541 | CTGGGAAGGG CCTTGGGGCT GCCTCCCCAA GCAGGCAGGC TGGTTGGGGT GCTGACTAGG |
| 601 | GCAGCTGGGG CAGAGGGAGG CAGGGGCAGG TGGGAGTAGG GTGGGGGCTG GGTGCAGCAG |
| 661 | CCGGGGACCT CTGGCCATCT TGGATTTTTT GGATGGATTT GTTTCCACAT TCCGATCGTT |
| 721 | AAGATTCAAG ATGAAACAAG ACACAGAGAC CCACACGACC CCCGAG |

CG locations, methylation status and restricting enzymes:

| | |
|---|---|
| 53: | BslI McrBC (half site) |
| 77: | AvaI BssKI HpaII Nt.CviPII ScrFI SmaI TspMI |
| 82: | BanI BbeI BsaHI HaeII HhaI HinPlI KasI NarI NlaIV SfoI |
| | McrBC (half site) |
| 137: | McrBC (half site) |
| 147: | McrBC (half site) |
| 157: | BanI BbeI BsaHI HaeII HhaI HinPlI KasI NarI NlaIV SfoI |
| | McrBC (half site) |
| 291: | BanI NlaIV Nt.CviPII |
| 322: | Hpy188iii Nt.CviPII |
| 367: | BslI Nt.CviPII |
| 662: | BssKI HpaII Nt.CviPII ScrFI |
| 713: | BsiEI Nt.CviPII PvuI |
| 717: | BfuCI BsiEI DpnI PvuI Sau3AI |
| 756: | McrBC (half site) |
| 763: | AvaI Nt.CviPII |

-----------------------------------------------------------------------------------
Sequence 8: vWA
Amplicon length = 751 bps

| | |
|---|---|
| 1 | AGATGATAGA TACATATGTT AGACAGAGAT AGGATAGATGATAGATACAT AGGTTAGATA |
| 61 | GAGATAGGAT AGATTATAAA TAGATACACA GGTTAGATAGATTAGACAGA CAGATAGATA |
| 121 | CATACATAGA TATAGGATAG ATAACTAGAT ACAATAGAGATAGATAGATA GATAGATAGA |
| 181 | TGATAGAGGA TAGATGATAA ATAGATATAT AGCTTAGATAGAGATAGGAT AGATGATAGA |
| 241 | TACATAGGAT AGATAGAGAC AGGATAGATG ATAAATAGATACATAGGTTA GATAGAGATA |
| 301 | GGACAGATGA TAAATACATA GGATGGATGG ATAGATGGATAGATAGATAG ATAGATAGAT |
| 361 | AGATAGATAG ATAGATAGAT AGACAGACAG ACAGACAGACAGATAGATCA ATCCAAGTCA |
| 421 | CATACTGATT ATTCTTATCA TCCACTAGGG CTTTCACATCTCAGCCAAGT CAACTTGGAT |
| 481 | CCTCTAGACC TGTTTCTTCT TCTGGAAGGT GGGAACTCTACCTTATAGGA TCAGTCTGAG |
| 541 | GAGTTCACAA AATAATAAGG GCAAAGTGCC CGGCACATTGTAGGAGACTA GTAATGTCTA |
| 601 | TAAAATGAGG GGCTTGAAGT AAATGATCCC TCTAGTTCTCTCTACTGCTA ACATTCTAAG |
| 661 | ACCTCCTTTA CATTAATTGT TCTCAAGCCA CATCTCCCTCCCCTACAGGA CTTCTATTTA |
| 721 | TTTCTGATCA ATTTCATGAG TACAAATAAG T |

CG locations, methylation status and restricting enzymes:

| | |
|---|---|
| 571: | BssKI HpaII Nt.CviPII ScrFI |

-----------------------------------------------------------------------------------
Sequence 9: FGA
Amplicon length = 945 bps

| | |
|---|---|
| 1 | ACTGAACATT TGCTTTTGAA ATTTACTATC TATGTACCGT TGGAAAATTT ACTTAATATC |
| 61 | TCTGAATTTT TTTTCTTCAA CTGTGGAGTG AGGAAAATAA TACCTACTTT TAGGTAGATG |
| 121 | ATGGATATAA CACTTTTCTC TGCATATAGT AGACACTCAG TGCATAACTA TCGCCTTCCT |
| 181 | TTTCCCTCTA CTCAGAAACA AGGACATCTG GGACCACAGC CACATACTTA CCTCCAGTCG |
| 241 | TTTCATATCA ACCAACTGAG CTCTAACATT TTTCTGCAGA AGCTGGATAT GCTGTACTTT |
| 301 | TTCTATGACT TTGCGCTTCA GGACTTCAAT TCTGCTTCTC AGATCCTCTG ACACTCGGTT |
| 361 | GTAGGTATTA TCACGGTCTG AAATCGAAAA TATGGTTATT GAAGTAGCTG CTGAGTGATT |
| 421 | TGTCTGTAAT TGCCAGCAAA AAAGAAAGGA AGAAAGGAAG GAAGGAGAAA GAAAGAAAGA |
| 481 | AAGAAAGAAA GAAAGAAAGA AAGAAAGAAA GAAAGAAAGA AAGAGAAAAA AGAAAGAAAG |
| 541 | AAACTAGCTT GTAAATATGC CTAATTTTAT TTTGGTTACA GTTTAATCTG TGAGTTCAAA |
| 601 | ACCTATGGGG CATTTGACTT TTGGATAATG TTATGCCCTG CAGCCTTCCA TGAATGCCAG |
| 661 | TTAAGATGTC CTAATAGCAA TTAGTAATCC CAAAGAAATA TAGAAGAAGA ACTTTCTTTG |
| 721 | GAATTTTAAA GGTGTAATTT GGAGTTAAAA TAGTTGGTTT GATTGCATTT CAATTATTTT |
| 781 | ATAACATCCT TAATCAAGGG ACTTGAACAT ATTGGATTTT CTTACTGATG AGCTTTTCTT |
| 841 | TTTAATCTAT AGATTTGAAA TGGTTCCTAA GCTGTTTTGG GTCAACAGGA TCACTCACTT |
| 901 | GCCAGCTAGT GTTGCATCAC TGATTTTAAA TGTCAAGTGT TTGTG |

CG locations, methylation status and restricting enzymes:

| | |
|---|---|
| 38: | Nt.CviPII |
| 172: | |
| 239: | |
| 314: | HhaI HinPlI McrBC (half site) |
| 356: | |
| 374: | McrBC (half site) |
| 385: | |

-----------------------------------------------------------------------------------
Sequence 10: D21S11
Amplicon length = 823 bps

| | |
|---|---|
| 1 | GTTGGCTGGG GCTCAGAGAG AACAAAAAGG CAGAGGAAAA ACAAATTTCC CCTCTCACTT |
| 61 | CTGGAGATGG AACACTTTTC TTCTGCTTTT GGACATCAGA AATCCAAGTT CTCTGGCCTT |
| 121 | TGGACTTTGG GACTTGTGCC AGCACCCTCC TGGGTTCCCT GGCCTTTGGC CTCAAACTGA |
| 181 | AGGTTACACT ATCAGCTTCC GTTGTTCTAA GGGCTTCAGA CTTGGACAGC CACACTGCCA |
| 241 | GCTTCCCTGA TTCTTCAGCT TGTAGATGGT CTGTTATGGG ACTTTTCTCA GTCTCCATAA |
| 301 | ATATGTGAGT CAATTCCCCA AGTGAATTGC CTTCTATCTA TCTATCTATC TGTCTGTCTG |
| 361 | TCTGTCTGTC TGTCTATCTA TCTATATCTA TCTATCTATC ATCTATCTAT CCATATCTAT |
| 421 | CTATCTATCT ATCTATCTAT CTATCTATCT ATCTATCTAT CGTCTATCTA TCCAGTCTAT |
| 481 | CTACCTCCTA TTAGTCTGTC TCTGGAGAAC ATTGACTAAT ACAACATCTT TAATATATCA |
| 541 | CAGTTTAATT TCAAGTTATA TCATACCACT TCATACATTA TATAAAACCT TACAGTGTTT |
| 601 | CTCCCTTCTC AGTGTTTATG GCTAGTAATT TTTTACTGGG TGCCAGACAC TAATTTTTAT |
| 661 | TTTGCTAAGT GGTGAATATT TTTTATATCC TTAAAAATAT TTTTGAGTGT TGATCTGGGT |
| 721 | AAAGTTAAGT TCAATATTGG AAAAATATTG ATTCTTTTGA GGATAGTTAT CTTCTAATTA |
| 781 | GTCTACCTGT TGCCCCATAA ATGGCATGAT TTTCCACTCT GTG |

CG locations, methylation status and restricting enzymes:

| | |
|---|---|
| 200: | Nt.CviPII |
| 461: | |

-----------------------------------------------------------------------------------
Sequence 11: D8S1179
Amplicon length = 824 bps

| | |
|---|---|
| 1 | TACTACAGCA AGAGCGCTTG AACCAGATGT AGGGGAGATA GCAGCTGGAG AGCATAACAG |
| 61 | AGGCACTGAC ATGTGAGCAG CTAACGAGGC CTTTTACAAG ACATCTGTGA CCACACGGCC |
| 121 | AAGTAGAAGA AAGCCGTTAA AAGCATCAAG GTAGTTAGGT AAAGCTGAGT CTGAAGTAAG |
| 181 | TAAAACATTG TTACAGGATC CTTGGGGTGT CGCTTTTCTG GCCAGAAACC TCTGTAGCCA |
| 241 | GTGGCGCCTT TGCCTGAGTT TTGCTCAGGC CCACTGGGCT CTTTCTGCCC ACACGGCCTG |
| 301 | GCAACTTATA TGTATTTTTG TATTTCATGT GTACATTCGT ATCTATCTGT CTATCTATCT |
| 361 | ATCTATCTAT CTATCTATCT ATCTATCTAT CTATTCCCCA CAGTGAAAAT AATCTACAGG |
| 421 | ATAGGTAAAT AAATTAAGGC ATATTCACGC AATGGGATAC GATACAGTGA TGAAAATGAA |
| 481 | CTAATTATAG CTACGTGAAA CTATACTCAT GAACACAATT TGGTAAAAGA AACTGGAAAC |
| 541 | AAGAATACAT ACGGTTTTTG ACAGCTGTAC TATTTTACAT TCCCAACAAC AATGCACAGG |
| 601 | GTTTCAGTTT CTCCACATCC TTGTCAACAT TTGTTATTTT CTGGGTTTTT GATAATAGCT |
| 661 | GTGAAAGGAA AATAAAAACT TGGGCCGGGC GCGGTGGCTC ACGCCTGTAA TCCCAGCACT |
| 721 | TTGGGAGGCC AAGGCGGGCA GATCTCAAGG TCGGGAGATT GAGACCATCC TGGCTAACAT |
| 781 | GGTGAAAACC CATCTCTACT AAAAATACAA AAACAAAAAA TTAG |

CG locations, methylation status and restricting enzymes:

| | |
|---|---|
| 15: | AfeI HaeII HhaI HinPlI McrBC (half site) |
| 85: | MwoI McrBC (half site) |
| 116: | BceAI EaeI McrBC (half site) |
| 135: | Nt.CviPII |
| 211: | |
| 245: | BanI BbeI BsaHI HaeII HhaI HinPlI KasI MwoI MarI NlaIV SfoI |
| | McrBC (half site) |
| 294: | BceAI BglI MwoI McrBC (half site) |
| 338: | BaeI |
| 448: | McrBC (half site) |
| 460: | McrBC (half site) |
| 494: | BsaAI HpyCH4IV TscI McrBC (half site) |
| 552: | McrBC (half site) |
| 686: | BssKI HpaII Nt.CviPII Sau96I ScrFI |
| 690: | BstUI HhaI HinPlI McrBC (half site) |
| 692: | BstUI HhaI HinPlI |
| 702: | McrBC (half site) |
| 735: | Cac8I McrBC (half site) |
| 752: | Hpy188iii |

-----------------------------------------------------------------------------------
Sequence 12: D18S51
Amplicon length = 927 bps

| | |
|---|---|
| 1 | CATGCCACTA AGCTGTACAC TGAAAAACGG TTAACATGAT AAATTTTATG TTACATACAT |
| 61 | TTTACCACAA TTTAAAAAAA TTATTAAAAA ATACTAACAA TAGGCCAAGC GTGATGGCTC |
| 121 | ACACCTGTAA TCCCAGCACT TTGGGAGGCT GAGACAGGTG GATCAATTGA GCTCAGGAGT |
| 181 | TTGAGACCAG CCTGGGTAAC ACAGTGAGAC CCCTGTCTCT ACAAAAAAAT ACAAAAATTA |
| 241 | GTTGGGCATG GTGGCACGTG CCTGTAGTCT CAGCTACTTG CAGGGCTGAG GCAGGAGGAG |
| 301 | TTCTTGAGCC CAGAAGGTTA AGGCTGCAGT GAGCCATGTT CATGCCACTG CACTTCACTC |
| 361 | TGAGTGACAA ATTGAGACCT TGTCTCAGAA AGAAAGAAAG AAAGAAAGAA AGAAAGAAAG |
| 421 | AAAGAAAGAA AGAAAGAAAG AAAGAAAGAA AGAAAGAAAA AGAGAGAGGA AAGAAAGAGA |
| 481 | AAAAGAAAAG AAATAGTAGC AACTGTTATT GTAAGACATC TCCACACACC AGAGAAGTTA |
| 541 | ATTTTAATTT TAACATGTTA AGAACAGAGA GAAGCCAACA TGTCCACCTT AGGCTGACGG |
| 601 | TTTGTTTATT TGTGTTGTTG CTGGTAGTCG GGTTTGTTAT TTTTAAAGTA GCTTATCCAA |
| 661 | TACTTCATTA ACAATTTCAG TAAGTTATTT CATCTTTCAA CATAAATACG CACAAGGATT |
| 721 | TCTTCTGGTC AAGACCAAAC TAATATTAGT CCATAGTAGG AGCTAATACT ATCACATTTA |
| 781 | CTAAGTATTC TATTTGCAAT TTGACTGTAG CCCATAGCCT TTTGTCGGCT AAAGTGAGCT |
| 841 | TAATGCTGAT CAGGTAAATT AAAAATTATA GTTAATTAAA AGGGCATAAA TGTTACCTGA |
| 901 | CTCAATAAGT CATTTCAATT AGGTCTG |

CG locations, methylation status and restricting enzymes:

| | |
|---|---|
| 28: | McrBC (half site) |
| 110: | McrBC (half site) |
| 257: | BsaAI HpyCH4IV PmlI TscI McrBC (half site) |
| 598: | McrBC (half site) |
| 629: | |
| 709: | McrBC (half site) |
| 826: | BslI |

-----------------------------------------------------------------------------------
Sequence 13: D3S1358
Amplicon length = 731 bps

| | |
|---|---|
| 1 | CTGGTTTTGG TGGAATTGAC TCCCTCTGTC ACAAACTCAG CTTCAGCCCA TACCCTGAGC |
| 61 | CATAGACCTA TCCCTCTAAT GCATTGTACT AGTCTCAGGG CTAATAACAA GGGAGAGGTG |
| 121 | TCAAAGGGCC AGTTCCACCT CCACCACCAG TGGAAAAGCT ATTCCCAGGT GAGGACTGCA |
| 181 | GCTGCCAGGG CACTGCTCCA GAATGGGCAT GCTGGCCATA TTCACTTGCC CACTTCTGCC |
| 241 | CAGGGATCTA TTTTTCTGTG GTGTGTATTC CCTGTGCCTT TGGGGGCATC TCTTATACTC |
| 301 | ATGAAATCAA CAGAGGCTTG CATGTATCTA TCTGTCTATC TATCTATCTA TCTATCTATC |
| 361 | TATCTATCTA TCTATCTATC TATCTATCTA TGAGACAGGG TCTTGCTCTG TCACCCAGAT |
| 421 | TGGACTGCAG TGGGGGAATC ATAGCTCACT ACAGCCTCAA ACTCCTGGGC TCAAGCAGTC |
| 481 | CTCCTGCCTC AGCCTCCCAA GTACCTGGGA TTATAGGCAT GAGCCACCAT GTCCGGCTAA |
| 541 | TTTTTTTTTT TAAGAGATGG GGTCTCGCTG TGTTCCCCAG CCTTGTCTTA AACTCCTGGC |
| 601 | CTCAAGTGAT CCTCCCATCT CAGCCTTCCA AAGTGCTGAG ATTACAGCAG AGGCTTTTAA |
| 661 | GTCAAAGCTT TCCCTGCTAG GACAAGCCCT AGTTAAAGTC CTGGAGCACT GGCCACTGCA |
| 721 | GCTGCACTTG G |

CG locations, methylation status and restricting enzymes:

| | |
|---|---|
| 534: | HpaII Nt.CviPII |
| 566: | BsaI BsmAI Nt.BsmAI |

-----------------------------------------------------------------------------------
Sequence 14: Penta D
Amplicon length = 1026 bps

| | |
|---|---|
| 1 | CCTACTCGGG AGGCTGAGGC AGGAGAATCG CTTGAACCCA GGAGGGGGCG ACTGCAGTGA |
| 61 | GCCGAGATCG TGCCACTGCA CTCCAGCCTG GGTGACAGAG CGAGACTCCA TCTCAAAAAA |
| 121 | AAAAAAAAAA AAACAGAATC ATAGGCCAGG CACAGTGGCT AATTGTACCT TGGGAGGCTG |
| 181 | AGACGGGAGG ATCGAGACCA TCCTGGGCAC CATAGTGAGA CCCCATCTCT ACAAAAAAAA |
| 241 | AAAAAAATTT TTTTTAAATA GCCAGGCATG GTGAGGCTGA AGTAGGATCA CTTGAGCCTG |
| 301 | GAAGGTCGAA GCTGAAGTGA GCCATGATCA CACCACTACA CTCCAGCCTA GGTGACAGAG |
| 361 | CAAGACACCA TCTCAAGAAA GAAAAAAAAG AAAGAAAAGA AAAGAAAAGA AAAGAAAAGA |
| 421 | AAAGAAAAGA AAAGAAAAGA AAAGAAAAGA AAAGAAAAAA CGAAGGGGAA AAAAAGAGAA |
| 481 | TCATAAACAT AAATGTAAAA TTTCTCAAAA AAATCGTTAT GACCATAGGT TAGGCAAATA |
| 541 | TTTCTTAGAT ATCACAAAAT CATGACCTAT TAAAAAATAA TAATAAAGTA AGTTTCATCA |
| 601 | AAACTTAAAA GTTCTACTCT TCAAAAGATA CCTTATAAAG AAAGTAAAAA GACACGCCAC |
| 661 | AGGCTAAGAG AAAGTACTTC TAATCACATA TCTAAAAAAG GACTTGTGTC CAGATTAAAG |
| 721 | AATTCTTACA CATCAATAAG ACAACCCAAT TAAAAATGGG CAAAAGATTT GAAGAGATAT |
| 781 | TTAACCAAAG AAAACATATA AATGTGTCCG GGCGCGATGG TAATCCCAGC ACTTTGAGAG |
| 841 | GCCGAGGCAG GCGGATCACT TGAGGTCAGG AGTTTAGGAC CAGTCTGGCC AACATGGTGA |
| 901 | AACCCTGTCT CTAATAAAAA TACAAAAATT AGCTGGGTGT GGTGGCGTAA GCCTGTAATC |
| 961 | CCAGCTGCTC AGGAGGCTGA GGCAGAAGAA TTGCTTGAAC CTGGGAGGTG GAGGCTGCAG |
| 1021 | TAAGCG |

CG locations, methylation status and restricting enzymes:

| | |
|---|---|
| 7: | AvaI Hpy188iii |
| 29: | HinfI TfiI |
| 49: | McrBC (half site) |
| 63: | Nt.CviPII |
| 69: | BfuCI DpnI Sau3AI |
| 101: | McrBC (half site) |
| 184: | McrBC (half site) |
| 193: | BfuCI DpnI Hpy188iii Sau3AI |
| 307: | |
| 461: | McrBC (half site) |
| 515: | |
| 655: | McrBC (half site) |
| 809: | BssKI HpaII Nt.CviPII ScrFI |
| 813: | BstUI HhaI HinPlI McrBC (half site) |
| 815: | BstUI HhaI HinPlI |
| 843: | Nt.CviPII |
| 852: | Cac8I EciI McrBC (half site) |
| 946: | McrBC (half site) |
| 1025: | McrBC (half site) |

-----------------------------------------------------------------------------------
Sequence 15: Penta E
Amplicon length = 977 bps

| | |
|---|---|
| 1 | CACATGTGGA CATTTCTTAT TTTCTCATAT TGGTGGTATG GCTCATTTAT GAAGTTAATA |
| 61 | CTGGACATTG TGGGGAGGCT GTGTAAGAAG TGTTAAAGGG GATCAGGGAT ACATTCACTT |
| 121 | CTCTTTTCCT TTGCTAGTTC TGTGGTCTTA AGCAAAGTAG CCTCAAACAT CAGTTTCCTC |
| 181 | TTTTATAAAA TGAGGAAAAT AATACTCATT ACCTTGCATG CATGATATAA TGATTACATA |
| 241 | ACATACATGT GTGTAAAGTG CTTAGTATCA TGATTGATAC ATGGAAAGAA TTCTCTTATT |
| 301 | TGGGTTATTA ATTGAGAAAA CTCCTTACAA TTTTCTTTTC TTTTCTTTTC TTTTCTTTGA |
| 361 | GACTGAGTCT TGCTCAGTCG CCCAGGCTGG AGTGCAATGG CGTGATCTCG GCTCACTTCA |
| 421 | ATCTCCACCT CCTGGGTTCA AGTGATTCTC CTGTTTCAGC CTCCAGAGTA GCTGGGATTA |
| 481 | CAGGTGCCTA CCACCACACC CAGCTAATTT TTTGTATTTT AGTAGAGACG GGGTTTCACC |
| 541 | ATGTTGCCCA GGCTGGTCTT GATCTCCTGA GCTCAGGTAA TACACCTGCA TCGGCCTCCC |
| 601 | AAAGTGCTAG GATTGCAGGC GTGAATCACC GCACCTGTCC ACAATTTTCT TGTTATTGGT |
| 661 | ACCCTTTCAT GTTGGTAAAA TGTATTTTAT TTTCTCTTAT CAAATAATTT TCAATGCAAT |
| 721 | GAGACGTCAA CTTTAAGCCC AAAGTAGACC AGTAGTAAAA CTAAGGCTGA AACCATTGAT |
| 781 | TGATTATTAC CATATATTGT CCTAAAATAT TCGGCTTTTA AAACATTTGG TTTCATTTTT |
| 841 | CATGATAAAA ATATGTAGCA TTTTTGCACT TTTAATTCAC TTTGTAGAGT TCTCAATCAT |
| 901 | TTCTAACACA TGCTTGGCAA TGACAAGCCA TTTGTGAAAG AGTTTTGCTG GCTTTAAAAT |
| 961 | ATATGCAAAT GTAATAT |

CG locations, methylation status and restricting enzymes:

| | |
|---|---|
| 379: | |
| 401: | McrBC (half site) |
| 409: | |
| 529: | McrBC (half site) |
| 592: | |
| 620: | Cac8I McrBC (half site) |
| 630: | AciI Nt.CviPII |
| 725: | AatII BsaHI HpyCH4IV TscI ZraI McrBC (half site) |
| 812: | |

-----------------------------------------------------------------------------------
Sequence 16: AMEL X
Amplicon length = 706 bps

| | |
|---|---|
| 1 | AGGTCTCCTC TTCTATACAG CACATTTGTT CAAACTAAAA ACAGACCTCA AGTATATTCT |
| 61 | GCACTATATA GATTTTTTTA AAGTAGCTTC AGTCTCCTTT AATGTGAACA ATTGCATACT |
| 121 | GACTTAATCT CTTCCTCTCT CTTCTCTTCC TTCACTCTCT CCCTTCCTCT CTCTTTCTAT |
| 181 | TCTCCTCCCC TCCTCCCTGT AAAAGCTACC ACCTCATCCT GGGCACCCTG GTTATATCAA |
| 241 | CTTCAGCTAT GAGGTAATTT TTCTCTTTAC TAATTTTGAC CATTGTTTGC GTTAACAATG |
| 301 | CCCTGGGCTC TGTAAAGAAT AGTGTGTTGA TTCTTTATCC CAGATGTTTC TCAAGTGGTC |
| 361 | CTGATTTTAC AGTTCCTACC ACCAGCTTCC CAGTTTAAGC TCTGATGGTT GGCCTCAAGC |
| 421 | CTGTGTCGTC CCAGCAGCCT CCCGCCTGGC CACTCTGACT CAGTCTGTCC TCCTAAATAT |
| 481 | GGCCGTAAGC TTACCCATCA TGAACCACTA CTCAGGGAGG CTCCATGATA GGGCAAAAAG |
| 541 | TAAACTCTGA CCAGCTTGGT TCTAACCCAG CTAGTAAAAT GTAAGGATTA GGTAAGATGT |
| 601 | TATTTAAAAC TCTTTCCAGC TCAAAAAACT CCTGATTCTA AGATAGTCAC ACTCTATGTG |
| 661 | TGTCTCTTGC TTGCCTCTGC TGAAATATTA GTGACTAAGT GGTATA |

CG locations, methylation status and restricting enzymes:

| | |
|---|---|
| 290: | McrBC (half site) |
| 427: | |
| 443: | AciI FauI Nt.CviPII |
| 484: | EaeI Nt.CviPII |

-----------------------------------------------------------------------------------
Sequence 17: AMEL Y
Amplicon length = 712 bps

| | |
|---|---|
| 1 | TTATTCTCCA ATATTTTGAA ATGTGAATAT TACAGTAATT TCCCTTGTCC AAATGAGAAA |
| 61 | ACCAGGGTTC CAAAGAGAGG AAATTATTTG CCCAAAGTTA GTAATTTTAC CTAATCTTTA |
| 121 | CATTTTACCG GATGGGATAG AACCAAGCTG GTCAGTCAGA GTTGACTTTT TGCCCTTTCA |
| 181 | TGGAACCTTC CTGAGCAGTG GTTCATGAAT GAATAAACTT ACAGCCATAT TTAGGAGGAA |
| 241 | AGAGTCAATC CGAATGGTCA GGCAGGAGGG TGCTGGAGCA ACACAGGCTT GAGGCCAACC |
| 301 | ATCAGAGCTT AAACTGGGAA GCTGATGGTA GGAACTGTAA AATTGGGACC ACTTGAGAAA |
| 361 | CCACTTTATT TGGGATGAAG AATCCACCCA CTATTCTTTA CAGAGCCCAG GGGACTGCTA |
| 421 | ATGCAAACAG TGATCAAAAT TAGTAAAGAG AAAAATTACC TCATAGCTGA AGTTGATATA |
| 481 | ACCAGGGTGC CCAGGATGAG GTGGTAGCTT TTATAGGGAG GAGGGGAGGA GAAGAGAAAG |
| 541 | AGAGAGGAAG GGAGAGTGTG AAGGAAGGGA AGAGAGAGTA AGAGATTAAG TCAATATGCA |
| 601 | ATTGTTAACA TTAAGAGAGA CTAAAATTAC TTTTAAAAAA TCTATATAGT ACAGAATATA |
| 661 | TTTGAGGTCT GTTTTTCGTT AAAACAAGTG TGCTATGTAG GAGAGGAGAC TT |

CG locations, methylation status and restricting enzymes:

| | |
|---|---|
| 129: | HpaII Nt.CviPII |
| 251: | Nt.CviPII |
| 677: | |

-----------------------------------------------------------------------------------
Sequence 18: D2S1338
Amplicon length = 840 bps

| | |
|---|---|
| 1 | ACAAGGCACG GAACTCACAC CCAGCCTCTC TCCATACAAC AGAATATGGG TTCTTGCGGA |
| 61 | GCTGGACTCT GCAGGAGTCT ATCTAATATG GACTCTGTGT CAATGACTCC TGGGCCTCCT |
| 121 | CTGATCACCC CATTAAAGTC CTTCGATTGC TTTGAGCCTC AAATCTATGT GACATCAATA |
| 181 | CGTTCATTTC TTCCTAGCAC TTAGAACTGT TTCTTGTTGA TACATTTGCT GGCTTCTTCC |
| 241 | CTGTCTCACC CCTTTTCCTA CCAGAATGCC AGTCCCAGAG GCCCTTGTCA GTGTTCATGC |
| 301 | CTACATCCCT AGTACCTAGC ATGGTACCTG CAGGTGGCCC ATAATCATGA GTTATTCAGT |
| 361 | AAGTTAAAGG ATTGCAGGAG GGAAGGAAGG ACGGAAGGAA GGAAGGAAGG AAGGAAGGAA |
| 421 | GGAAGGAAGG AAGGAAGGAA GGAAGGCAGG CAGGCAGGCA GGCAGGCAGG CAAGGCCAAG |
| 481 | CCATTTCTGT TTCCAAATCC ACTGGCTCCC TCCCACAGCT GGATTATGGG CCAGTAGGAA |
| 541 | TTGCCATTTT CAGGGTTTTG CTGTCACTGT AGTCAGGACC ATGAAGTCTT TAGGCACCTC |
| 601 | CACTCCACAC ACCCCCTGGT GAGAGCTCCC ATCTCCCTGT TCTGAAACAG CTCCCCAATA |
| 661 | TAGTACTGAT TCCGGTTAAA CTTGAACCCC TGCCCCTGCC CCTGCCCCTG ATTTACATGA |
| 721 | GGACACTGAG GCCCAGAGGG GTAAAGTGAC TGCCAGGGGT CACACAGCTA GAAAGTGGCG |
| 781 | GTGCCAGAAC TGGAAGGAGG CCCTCATTCC TGAGTCACGG CTTTTCCATA GCACAGCCTT |

CG locations, methylation status and restricting enzymes:

| | |
|---|---|
| 9: | McrBC (half site) |
| 57: | McrBC (half site) |
| 144: | |
| 181: | HpyCH4IV TscI McrBC (half site) |
| 392: | McrBC (half site) |
| 673: | HpaII Nt.CviPII |
| 779: | McrBC (half site) |
| 818: | BceAI McrBC (half site) |

-----------------------------------------------------------------------------------
Sequence 19: D19S433
Amplicon length = 780 bps

| | |
|---|---|
| 1 | ATGAAACTGG ACACAGAAAC CAGACCCCAG AGCACATACC GTATGAGTCC ATTGGTATGA |
| 61 | AGTTTAAAAA CAGATGGCAC TAGTCCAAAG GATTGGAAGT TGGAATAGTG GTTACCAGGA |
| 121 | CTGGGGGGAG GAAGGGATGG TGGATGGTGA ACAAAAGGAC CTTGGAGGGC TCCTGGGGTT |
| 181 | CTAGGAATCA ATCTTCCTTC TTTCCTTCCT TCCTTCCTTC CTCTTTCTCT CTTTCTTTCT |
| 241 | GTTTTTATTT CAATAGGTTT TTAAGGAACA GGTGGTGTTG GTTACATGAA TAAGTTCTTT |
| 301 | AGCAGTGATT TCTGATATTT TGGTGCACCC ATTACCCGAA TAAAAATCTT CTCTCTTTCT |
| 361 | TCCTCTCTCC TTCCTTCCTT CCTTCCTTCC TTCCTTCCTT CCTTCCTTCC TTCCTTCCTA |
| 421 | CCTTCTTTCC TTCAACAGAA TCTTATTCTG TTGCCCAGGC TGGAGTGCAG TGGTACAATT |
| 481 | ATAGCTTTTT GCAGCCTCAA CCTCCTGGGC TCAAGTGATC TTCCTGCCCC AGCCTCCTGA |
| 541 | GTAGCCAGGA CTACAGGAAT GTGCCAACAT GCCTGGCTAA TTTTAAAAAA TTTTTTATAG |
| 601 | AGAAGAGGTC TCACTATGTT GCCCAGACTA GACTTGAACT CCTTCCCTCA AGTGATCTTT |
| 661 | CTGCATCAGT CTTCCAAAGT GCTGGGATTG CAGGCATGAG CCACCTCACC CAGCCTTAGA |
| 721 | AATGTTCTGT TTCTTGACCT GAGAGCTGGA TATACAGGAT TGCTCACTTT GTGAAAATTC |

CG locations, methylation status and restricting enzymes:

| | |
|---|---|
| 40: | Nt.CviPII |
| 337: | Nt.CviPII |

-----------------------------------------------------------------------------------
Sequence 20: ACTBP2SE33
Amplicon length = 887 bps

| | |
|---|---|
| 1 | GTACTTCAGA GTCAGGATGC CTCTCTTGCT CTGGGCCTCC TTGCCCACAT AGGAGTCCTT |
| 61 | CTGACCCATG CCCACCATCA CTCCCTGGTG CCTAGGGTGC CCCACAATGG AGGGGAAGAC |
| 121 | GGCCTGGGGA GCCTTGCGCA TGCTGGAGCA GTTGTCGACG ACGACGAGCG CGGTGATAGC |
| 181 | ATCATCCATG GTGAGCTGGC GGCGGGTGCG GACGCAAGGC GCAGCGGCAA GGACAAGGTT |
| 241 | CTGTGCTCGC TGGGCTGACG CGGTCTCCGC GGTGTAAGGA GGTTTATATA TATTTCTACA |
| 301 | ACATCTCCCC TACCGCTATA GTAACTTGCT CTTTCTTTCC TTCCTTTCTT TCTTTCTTTC |
| 361 | TTTCTTTCTT TCTTTCTTTC TTTCTTTCTT TCTTTCTTTC TTTCTTTTTC TTTCTTTCTT |
| 421 | TCTTTCTTTC TTTCTTTCTT TCTTTCTCTT TCTTTCTTTC TCTTTCTTTC TTTTTCTTTC |
| 481 | TTTTTCTTCC TTCCTTCCTT TCTCTCTCTC TCTCTTTCTT TCTTTCTAAC TCTCTTTGTC |
| 541 | TCTTTCTTTC TTTCTTTTGA CGGAGTTTCA CTCTTGTCGC CCAGATTGGA GTGCAATGGC |
| 601 | ATGACCTCGG CTCACTGTAG CCTCCACCTC CCAGGTTCAA GCGATTATCC TGCCTCAGCC |
| 661 | TCCCTAGGAG CTGGAATTAC AGACGTGCAC CACCAAGCCT GGCTAATTTT TGTATTATTA |
| 721 | GTAGAGACGG GGTTTCACCT TGTTGGCCAG GCTGGTCTCG AACTCCTGAC CTCAGGTGAC |
| 781 | CCACCTGCCT TAGGCTCCCA AAGTCCTGGG ATTATAGGCA TGAGCCACAG TGCCCAGCCT |
| 841 | TCTTTTCATT TAATACTATA GTAGTGTGAT CCTCTCTACC TATTACA |

CG locations, methylation status and restricting enzymes:

| | |
|---|---|
| 120: | BceAI McrBC (half site) |
| 137: | FspI HhaI HinPlI McrBC (half site) |
| 156: | AccI HincII HincII Hpy166ii Hpy99I SalI SalI-HF |
| 159: | AccI HincII Hincli Hpy166ii Hpy99I SalI SalI-HF McrBC(halfsite) |
| 162: | Hpy99I McrBC (half site) |
| 165: | Hpy99I McrBC (half site) |
| 169: | BstUI HhaI HinPlI McrBC (half site) |
| 171: | BstUI HhaI HinPlI MwoI |
| 200: | Cac8I Fnu4HI MwoI McrBC (half site) |
| 203: | Fnu4HI MwoI McrEC (half site) |
| 209: | MwoI McrBC (half site) |
| 213: | HgaI McrBC (half site) |
| 220: | HhaI HinPlI McrBC (half site) |
| 225: | Fnu4HI MspAlI TseI McrBC (half site) |
| 248: | Cac8I MwoI |
| 259: | BstUI HgaI McrBC (half site) |
| 261: | BstUI HgaI MwoI |
| 268: | AciI BstUI MspAlI MwoI Nt.BsmAI Nt.CviPII SacII |
| 270: | AciI BstUI MspAlI MwoI SacII McrBC (half site) |
| 314: | AciI Nt.CviPII |
| 561: | McrBC (half site) |
| 578: | |
| 608: | |
| 642: | BcgI McrBC (half site) |
| 684: | HpyCH4IV TscI McrBC (half site) |
| 728: | McrBC (half site) |
| 759: | BsaI BsmAI Hpy188iii Nt.BsmAI |

-----------------------------------------------------------------------------------
Sequence 21: D10S1248
Amplicon length = 720 bps

| | |
|---|---|
| 1 | TTCTGTTTTG CGGTGGTTCC TAGTATGGTA CCTGGCCAAG GGCACACTAG ATCTTTGTCA |
| 61 | AGGTAATGAC TACTTTTTAT TAAATGCTTT CCATGTATCA AGTTCTGTGC CAAGCACTTG |
| 121 | ACATATATCA TTTTATTTTA TCCCGTGAAG TAGTTATTGG TATCTTCATT TACAAATAAA |
| 181 | AAAACAAGCT TAGTACTTAA CTCACTGCCT TGAACATAAT TATTGCTTTA AAGGTAGCTA |
| 241 | GGATTCTTAA TAGCTATTAT TACCAAAGCA TGAACAATCA GTAAAAAGCA AACCTGAGCA |
| 301 | TTAGCCCCAG GACCAATCTG GTCACAAACA TATTAATGAA TTGAACAAAT GAGTGAGTGG |
| 361 | AAGGAAGGAA GGAAGGAAGG AAGGAAGGAA GGAAGGAAGG AAGGAAGGAA ATGAAGACAA |
| 421 | TACAACCAGA GTTGTTCCTT TAATAACAAG ACAAGGGAAA AAGAGAACTG TCAGAATAAG |
| 481 | TGTTAATTAT AATATCCAGG GGTGGGATAC AGAGGTTTTA GCATCTGCTC TTTGCCAAGC |
| 541 | ACTGCACTTA TTCCTGAGGA ATACCTGAGG GAAAAAGTAT GGTTTCTCAC AGGATCTAGT |
| 601 | TGGACTGGAA ATATGACATT CATATTGGAA TCCAGTGTCT TTTTCTGAAA AAGAGAGTTC |
| 661 | GTTCCAAGCT TAGCTCACAT GCAAGCTAAG ACAACCACTA GAAATTACTC TCCCCAGGGC |

CG locations, methylation status and restricting enzymes:

| | |
|---|---|
| 11: | McrBC (half site) |
| 144: | Nt.CviPII |
| 660: | |

-----------------------------------------------------------------------------------
Sequence 22: D1S1656
Amplicon length = 780 bps

| | |
|---|---|
| 1 | GTCATGCCTA CAGTGTAACG GGAATTGACC AGGTAGGCGA CTTGAACTCC AACTGCAGGC |
| 61 | TATGGGGAGA CATGTGACAA TGCTAATCCC TTAGGCATTT ATTCAGTGCA TTGCAGTTTA |
| 121 | AATGTCTGCC TTTCAGGCAT TTCAGAGATT ATGTCACCTA AAGAGGCAGG CTGGAATTCA |
| 181 | AAACGGCAAG CCAGGAAAGA GAGAAACCAT GTGATTCCAC CGCAGCACAA AACTCGTTTA |
| 241 | GCAGCTGTAA GCGCCTGGTC TTTGTTTATT TTTAATTTCC TTTCTTTCCC AATTCTCCTT |
| 301 | CAGTCCTGTG TTAGTCAGGA TTCTTCAGAG AAATAGAATC ACTAGGGAAC CAAATATATA |
| 361 | TACATACAAT TAAACACACA CACACCTATC TATCTATCTA TCTATCTATC TATCTATCTA |
| 421 | TCTATCTATC TATCTATCTA TCTATCTATC TACATCACAC AGTTGACCCT TGAGCAACAC |
| 481 | AGGCTTGAAC TTATATGGGG ATTTTCTTCC ATCTCTACCA CCCCTGAGAC AGCAAGACCA |
| 541 | ACTCCTCCTC CTCCTTCTCA GCCTACTCAA CATGAAGATA ATAAGGATGA AGACCTTTAC |
| 601 | AATGACCCAG TTCCACTTAA TAAATAGTAA ATGTATTTCC TCTTCCCTAT GATTTTCTTG |
| 661 | ATAACATTTC TTTTCTCTGG CTTATTTATT GTAAGAATAC AGTATATAAT ATAAATAATT |
| 721 | ATAAAACATG TTAATTGGTT CTTTACGTTA TCGATAAGAC TTCTGGTCAA TGGTAGGCTA |

CG locations, methylation status and restricting enzymes:

| | |
|---|---|
| 19: | McrBC (half site) |
| 38: | McrBC (half site) |
| 184: | BceAI McrBC (half site) |
| 221: | AciI Nt.CviPII |
| 235: | |
| 252: | HaeII HhaI HinPlI MwoI McrBC- (half site) |
| 746: | HpyCH4IV TscI McrBC (half site) |
| 752: | BspDI ClaI |

-----------------------------------------------------------------------------------
Sequence 23: D22S1045
Amplicon length = 780 bps

| | |
|---|---|
| 1 | GAGCCCAAGT TTAAACCCAG GCCCTCTGTG TCCCCCTACA GGGTGACTGC ATCTCCGAGT |
| 61 | CCTGGCTTGT CATGCCTGAC AGAGGGCTGC CGAGTGAGCA GCTTAAGGCA TCCTGCCACT |
| 121 | GTGCAGCTGC CAACCCTACA GCCCGGCAGC CCTGCGGGAG GAAGCTCTAG TGCAGGCCTC |
| 181 | TTAGGATCTG GGGTCCAGGA TGCTGATTTC AGGGCCGGGA CCTTGGGCAC CGTCCCTCTG |
| 241 | GTCTGCATAA GACCCACTAT GGGCAAACCT TAAACCTGAT CGTTGGAATT CCCCAAACTG |
| 301 | GCCAGTTCCT CTCCACCCTA TAGACCCTGT CCTAGCCTTC TTATAGCTGC TATGGGGGCT |
| 361 | AGATTTTCCC CGATGATAGT AGTCTCATTA TTATTATTAT TATTATTATT ATTATTATTA |
| 421 | TTATTATTAC TATTATTGTT ATAAAAATAT TGCCAATCAT ACATTCGCGT GATCACTCAC |
| 481 | ACTGTGCCGG GCACTCTTGA GAGCACTTTA CATATATTGT CTCATTTAAT TCTCTCAACT |
| 541 | TGGGCACAGG CACTGTCACT ATTTCCATTC TACAGCTGAG GAGACTGAAG CACAGAGAGC |
| 601 | CTTAGGGACT TGCCTGAGGT CACACAGCTA AGAAATGGTG GAGCCAGGAT CAGAAACCAG |
| 661 | GCCACCTACA GAGCTCCCTG CAAGGGGAAC AGCATCCGGT TCCAGAGGCT GTGATTTTAT |
| 721 | CAGCTACACT GTGTGACTCC ATCTTCACAC TCTCCTGCCC CTCAAGAAGA CATATAACCT |

CG locations, methylation status and restricting enzymes:

| | |
|---|---|
| 56: | BslI Nt.CviPII |
| 91: | MwoI Nt.CviPII |
| 144: | BssKI HpaII Nt.CviPII ScrFI |
| 155: | BslI McrBC (half site) |
| 216: | BssKI HpaII Nt.CviPII Sau96I ScrFI |
| 231: | BanI NlaIV Nt.CviPII |
| 281: | BfuCI DpnI Sau3AI |
| 371: | Nt.CviPII |
| 466: | BstUI |
| 468: | BstUI McrBC (half site) |
| 488: | BssKI HpaII Nt.CviPII ScrFI |
| 697: | HpaII Nt.CviPII |

-----------------------------------------------------------------------------------
Sequence 24: D2S441
Amplicon length = 780 bps

| | |
|---|---|
| 1 | ATGAAGAGAT GGTCAGGCGA GGTATGGGGG AAGGGGCGTG GAGCTTCCAT GTCCTCCCTG |
| 61 | GGCGCCACCC TCCAGGAACC TCCACGTGTT CAGCTATACA GAAGCTTCCT GAACCCAGTC |
| 121 | CTCTTGGGGT TTGAGGGAAG CTTCATGACA TCAGCATTCC TTCCTCCAGG GTATTAATGG |
| 181 | GACCCTCTCT GAAGAGATTC TTAAGACCCA CGGCCAGAAA GTTGGGTAAA GACTAGAGTC |
| 241 | CTGCCTTGGG GCAGGTGAAA GGAGTGCAAG AGAAGGTAAG AGAGATTCTG TTCCTGAGCC |
| 301 | CTAATGCACC CAACATTCTA ACAAAAGGCT GTAACAAGGG CTACAGGAAT CATGAGCCAG |
| 361 | GAACTGTGGC TCATCTATGA AAACTTCTAT CTATCTATCT ATCTATCTAT CTATCTATCT |
| 421 | ATCTATCTAT CTATATCATA ACACCACAGC CACTTAGCTC CAATTTAAAA GATTAATCAT |
| 481 | AAACATTTGG GAAGGAGAGT GAAGATTTTT GTGATGTTAA ATAAGAATGA TTATACTAAA |
| 541 | AACCAAAATA ATATGTTATT TATGGCTGGG TGTGGTGGCT TAAGCCTGTA ATCCCAGAAC |
| 601 | TTTGGGAGGC CAAGGCTTGT GGATCACTTG AGCCCAGAAG TTCAAGACCA GCCTGGGCAA |
| 661 | CATAGGGAGA CCCTGTCTCT ACAAAAAATT TTAAAATTAG CTGGACATGA TGGCACGCAC |
| 721 | CCGTAGTCTC AGCTACTCAG GAGGCTCACG CCACTGCATT CCAGTCTGGG TAACGCACAC |

CG locations, methylation status and restricting enzymes:

| | |
|---|---|
| 18: | McrBC (half site) |
| 37: | McrBC (half site) |
| 63: | BanI BbeI BsaHI HaeII HhaI HinPlI KasI NarI NlaIV SfoI |
| | McrBC (half site) |
| 85: | BsaAI HpyCH4IV PmlI TscI McrBC (half site) |
| 211: | BceAI EaeI McrBC (half site) |
| 716: | Cac8I McrBC (half site) |
| 722: | Nt.CviPII |
| 749: | McrBC (half site) |
| 774: | McrBC (half site) |

-----------------------------------------------------------------------------------
Sequence 25: D12S391
Amplicon length = 780 bps

| | |
|---|---|
| 1 | GTCAGGAGTT CGAGACCAGC CTGGCCAACA TGGCGAAACC CTGTCTCTAC TAAAAATACA |
| 61 | AAAAAATTAG CTGGGCATGG TGGTGTGTTC CTGTAACCCC AGCTACTCAG GAGGCTGAGG |
| 121 | CAAGAGAATC GCTGGAACCC AGGAGGTGGA AGTTGCAGTG AGCTGAGATT GCACCACTGC |
| 181 | ACTCCAGTGT GGGCAACAGA GCGAGACTCT GTCTCAGAAA AAAAAAAGAA TACATGAAAT |
| 241 | CAGAGAAACT CAAATTGTGA TAGTAGTTTC TTCTGGTGAA GGAAGAAAAG AGAATGATAT |
| 301 | CAGGGAAGAT GAAAAAAGAG ACTGTATTAG TAAGGCTTCT CCAGAGAGAA AGAATCAACA |
| 361 | GGATCAATGG ATGCATAGGT AGATAGATAG ATAGATAGAT AGATAGATAG ATAGATAGAT |
| 421 | AGATAGACAG ACAGACAGAC AGACAGACAG ACAGATGAGA GGGGATTTAT TAGAGGAATT |
| 481 | AGCTCAAGTG ATATGGAGGC TGAAAAATCT CATGACAGTC CATCTGCAAG CTGGAGACCC |
| 541 | AGGGACACTA GGAGCATGGC TCAGTCCAGG TCTAAAAGCC AAAAAACCAG GGAAACTGAT |
| 601 | GGTGTAATTA TCCATCCCAG GTGGAAGGCC TGAGAACCTG GAGTGCCCCT GGTATAAGTC |
| 661 | CCAGAGTACA AAGACAGGAG AGCCTGGAGT TCTGACTTCC AAGGGCAGAA GAATGTGTCG |
| 721 | CAGCTCCAGG AGAGAGAGAG AAAGAATTTC TTTCCTCCGC CTTTTGATTC TATCTGGGGG |

CG locations, methylation status and restricting enzymes:

| | |
|---|---|
| 11: | Hpy188iii |
| 34: | BglI MwoI McrBC (half site) |
| 130: | HinfI TfiI |
| 202: | McrBC (half site) |
| 719: | |
| 758: | AciI Nt.CviPII |

-----------------------------------------------------------------------------------
Sequence 26: ADD6 (from ncbi accession NT 022135)

| | |
|---|---|
| 16664701 | cttgaacctg agaggcagag gttgcagtga gccgagacca tgccattgca ctccagcctg |
| 16664761 | ggcaatagag taaaactcca tcctcccgct ccaaaaaagt agacaacgtc catgaggtga |
| 16664821 | tgaggaaggg gttatcgtgt gttgcttgct gagaacagga cccccagact caccgtgtcg |
| 16664881 | acgccggcca gcagcatctc agtcacgttg gcgtagatct cctgcagcgt cagagcctgg |
| 16664941 | ctaaggaaga ggtatgtgag aagtcccccg ctcaccctcc ggcctcggtc catttggtac |
| 16665001 | tgtatgtccc tcaacttgtt gtcaacatga atttggcctg tttgaaaaca gtatttcttt |
| 16665061 | tgaaaggagt ttgggttgag aatcatcttt tcagtctcaa agccctctgt cctcccagta |
| 16665121 | gcttaactaa accagtggca ggtgacagag ggtaaggaaa cccaatttat ctaacgtcaa |
| 16665181 | cctgggagtt tcactcatac acttgcttat gtaaatgaat gaaaagttaa aagacaagct |

Sequence 27: ADD10 (from chromosome 17:3477839-3478292)

| | |
|---|---|
| 1 | AGAGAGCCCA GGAGACAGGC AGAAAGGAAG GCATGTGACC GGATCACAAT CATCAGCTCT |
| 61 | CTGCTGTCCT CTTTGGGAAG GGTTTTAGTA TTAAAAGGAC ATTTATTCTC ATTAATGCAA |
| 121 | AATTAAGGAG TTTTAAAAGC TTTTACAACC TAGACTCCCT CTGAGAGGTT AGCCTTGACA |
| 181 | CCCTAATCGC CTTCTGCTCC CGCCACTGCT CGGTGCCAAG CAGCTCCCAC GGCCCCGGCG |
| 241 | GGTCTGATGA TAGCCGGACA GGAGGGAGGA AGGGGAGGAG GAAGAGCCTG CATCAGCTCC |
| 301 | TACGATTGCC CAGCCCCATC CTGGGAGTGA TTAAACGGTG CATCACCAAA TGCCAGTCCC |
| 361 | ACTGACAGGC AGGTCACCGT GCACTTCAGG GCACTCTAAA TTGCCGACTC TCCATGTAGA |
| 421 | GAGGGATGAA TCCAATATTG AAATCCTCAT AACTACAGCC CCCCAAAGTA GCCGTCCATC |
| 481 | TTCTGCTTAA AATGTTGATC TGTAGTAAAA TGTTGATTTT GTTGAAGCTG AGTGATG |

Sequence 28: ADD17 (from chromosome 1: 50149332-50149574)

| | |
|---|---|
| 1 | TTGAACCTAG GAGATGGAGG TTGCAGTGAG CTGCGATCAT GCCACTTCCC TCCAGGCTGG |
| 61 | GCAACAGAGC GAGACTCCAT CTCAAAAAAA CAAAAAAGAA AACCAACCTT TTGAATGTAG |
| 121 | GGGAAACTTT TCAAAGGATA TCTAGTTTTC AATTACAGTA AACTTGTGGA AGGGAGGTTC |
| 181 | AGAGTTGAGA TTGAGATTAT AGATTTTGCT GATGATAAAC CATGAGTTCC AGAGGACATA |
| 241 | GTAGACTATT CTGGGCAGTT ATACAGGGGT GGATGGAATG TGGGAGTGGG GTTGTATAGT |
| 301 | GCCATAAAGA AATGAGAGTC CGGATTAAAA ATAATGAGCT GGACTCGCGA GCCTTTTGTA |
| 361 | ACTGAAATAA ATAGAAAAAT AAGAAATACA TTATTTCTGT GATTGTTGAG AGGAAGAAAT |
| 421 | GGTGGAAATC TTGTGAGAAG CACACTGAGC TCTAGCACCA CCTCTTCACT CCTACAGATG |
| 481 | GTGGAATAAA CGGCAGGCAA GTTCAAAATC ACATATAGTC ATTATTGCAA GATAGTTCTA |
| 541 | TGGATATAGA TACTACATAC AATATAAATC ATGCTCATTG AATGGTTCAG TGGAAACTAC |
| 601 | TCTGAACTT |

Sequence 29: Hypo23(from ncbi accession NM_004907)

| | |
|---|---|
| 123161 | GAGTTTGGGA AGGGTATTTT AGGGGGGAAT AACTTTTGAG TTCCCAGCGT GCGGGGGAAG |
| 123221 | GGCGGGACGG GAGGGTGTCC CAAGGCCTGA GAAGATCAGT GTGGGGCAGG GGTCAGGAAT |
| 123281 | AACCTGGGAG GGGGCCTTGT ATGGGGGAAA TAATTGGGAA GAGGAGAGAT GGGATGAAGG |
| 123341 | GGGCCTCAGC GGGTCGTCTC CTGTGTATGC AGGGTCGTTC TGCAGCGTCT CTGGGAGATG |
| 123401 | GCGTCCCTGG GAGCCCTCAG GTCGCCCCTA CCCGCTGCGG GGTGCTTTCC TGGCGTCACG |
| 123461 | CCTTCCTGGC CCCTGGAGGG AAGGAAGTGA AACTCTCCTC TTCCCCCACC CGGCTGGAAT |
| 123521 | GCGAGTCAGG AAGCCTGGGG CTCCAGCCTG CTCCGGCTGC CCGGGTCGGG GATGGGGAGG |
| 123581 | GGCGTGGCCG GAGCGCAAAG CCCCGCCCCT CCGCGCCCCC CCCCCGGAAG CCCCGCCGCC |
| 123641 | GGCCGCTAAG GCGATCACGG GCCCTGTCCT AATATGGGCA ACCGGAAGCG GCCCGCGCGA |
| 123701 | CTGCCCTACG TCACTCCGTC CAAATTTAGT TGTGGAAGTC AGCGGGCGCT GGTGGCGGGA |
| 123761 | AGGCGCCGCG AGCCAGTGCG GGCGGAAAGG GGGCGGGGGG CGCACCACCC CTTAAAGGGC |
| 123821 | CCGCACCAGG AATGAATGGA GCCATTCGAA CAATTCTGCA TCCTATTTTT GGAGGAAGTG |
| 123881 | GAATTAGTAT TT |

Sequence 30: Hypo28 (from chromosome 5: 85949232-85949719)

| | |
|---|---|
| 949232 | TTTATTTTTA AAAAAGAAAG AAAGAAGAGA AAAGGGATGG GTTTATTGTC CTTTTCAACA |
| 949292 | GACTAGAGTA TACGGGGTGA AACTGCTTCA CTTGATTCAA TAAAATCGTT TCCGGTAACA |
| 949352 | GGCCCCAGGA ATCCTAGACC TAAGCCTGGC GCGAAACTAC ATTTCCCACA ATCCTTCGGG |
| 949412 | GGCTGATAAG GCTCCGCAAT GGTCTGAACT ACAATTCCCA CAATCCAGGG CGATTTCCGC |
| 949472 | TTTGTCGCGT TTCCTCAAGG CTCCGCCCCA TTTCCCATCT TTCTTTTCAG TCCTTGCGCA |
| 949532 | CCGGGGAACA AGGTCGTGAA AAAAAAGGTC TTGGTGAGGT GCCGCCATTT CATCTGTCCT |
| 949592 | CATTCTCTGC GCCTTTCGCA GAGCTTCCAG CAGCGGTATG TTGGGCCAGA GCATCCGGAG |
| 949652 | GTTCACAACC TCTGTGGTCC GTAGGAGCCA CTATGAGGAG GGCCCTGGGA AGGTTAGTGT |
| 949712 | GTAAGGGG |

Sequence 31: Hypo33 (from chromosome 6: 34211067-34211314)

| | |
|---|---|
| 211067 | ACCCTCATTT CACATTTCAC CCCTTCCTCA AAATGCTCCC TTCATATTAC CTCCTCAGAA |
| 211127 | ACCAAGAATA TGGCTACTAA TTCTCCCTGG CCCCATGCTG CAGGTGAACC GGTAGCCCAG |
| 211187 | AGGTATCACA TAATTCTCCC AAAGTCACAC AGCAAATCAA GATGCATCCA GGACTAGAAG |
| 211247 | CCATGTCAGC CACACTGGGA AGCCCCAGCG AAGCTGACAG AAAGTTTCAT AATACCACCC |
| 211307 | TCTCCCCT |

Sequence 32: OCA2 sequence: (from chromosome 15: 25276967-25277446)

| | |
|---|---|
| 6967 | aaacacccca gtctgaaaat aaccatagtt tgttgctctt acgagtgaaa atgctatttc |
| 7027 | atacacgaag ctttgtcctt cagcacccaa gatttaagga taattatgga tgaatattat |
| 7087 | ggattcattt taaatccttt ggcaaatctg ctctgggggc ttctctgtca gaaggtctct |
| 7147 | ccttcccaac tctaagaaac gttattccta tgcaaatgct gctgagtcaa gacggggagg |
| 7207 | gaagtgcaga gagaagggct ggtggcatgg tcagtaagtc atgagggtga gattaggggt |
| 7267 | gacacactgc ttgccaacgt aggagaaggc tctgccctca cctagcaggt ctgatggaag |
| 7327 | ccccttattc cgtccttcct gccgggttcc accgagatcc aaaaaggaat gctgtgtagg |
| 7387 | agcacatgat atgtgataaa tgagagaaag gtcaaacatt taaggaacgc ccagagaaag |

What is claimed is:

1. A method for identifying the source of a DNA sample, comprising:
(a) digesting the DNA sample with a methylation-sensitive and/or methylation-dependent restriction endonuclease;
(b) amplifying the digested DNA with at least a first and a second restriction locus, thereby generating an amplification product for each restriction locus;
(c) determining the intensity of the signal of each amplification product;
(d) calculating at least one methylation ratio between the intensity of the signals corresponding to the two restriction loci;
(e) comparing the methylation ratio calculated in step (d) to a set of reference methylation ratios obtained from DNA of known tissues and/or cell types; and
(f) identifying the source of the DNA sample based on determining the likelihood of each tissue and/or cell type being the source of the DNA, wherein the tissue/cell type with the largest likelihood is determined to be the source of the DNA sample.

2. The method of claim 1, wherein said source is a tissue or cell type.

3. The method of claim 1, wherein DNA digestion and amplification are performed in a single biochemical reaction in a single test tube.

4. The method of claim 3, wherein said single test tube comprises DNA template, digestion and amplification enzymes, buffers, primers, and accessory ingredients.

5. The method of claim 4, wherein said single test tube is closed and placed in a thermal cycler, where the single reaction takes place.

6. The method of claim 1, wherein said methylation-sensitive restriction endonuclease is unable to cut or digest DNA if its recognition sequence is methylated.

7. The method of claim 1, wherein said methylation-sensitive restriction endonuclease is selected from the group consisting of AatII, Acc65I, AccI, AciI, AClI, AfeI, AgeI, ApaI, ApaLI, AscI, AsiSI, AvaI, AvaI, BaeI, BanI, BbeI, BceAI, BcgI, BfuCI, BglI, BmgBI, BsaAI, BsaBI, BsaHI, BsaI, BseYI, BsiEI, BsiWI, BslI, BsmAI, BsmBI, BsmFI, BspDI, BsrBI, BsrFI, BssHII, BssKI, BstAPI, BstBI, BstUI, BstZ17I, Cac8I, ClaI, DpnI, DrdI, EaeI, EagI, Eagl-HF, EciI, EcoRI, EcoRI-HF, FauI, Fnu4HI, FseI, FspI, HaeII, HgaI, HhaI, HincII, HincII, HinfI, HinP1I, HpaI, HpaII, Hpy166ii, Hpy188iii, Hpy99I, HpyCH4IV, KasI, MluI, MmeI, MspA1I, MwoI, NaeI, NarI, NgoNIV, Nhe-HFI, NheI, NlaIV, NotI, NotI-HF, NruI, Nt.BbvCI, Nt.BsmAI, Nt.CviPII, PaeR7I, PleI, PmeI, Pm1I, PshAI, PspOMI, PvuI, RsaI, RsrII, SacII, SalI, SalI-HF, Sau3AI, Sau96I, ScrFI, SfiI, SfoI, SgrAI, SmaI, SnaBI, TfiI, TscI, TseI, TspMI, and ZraI.

8. The method of claim 7, wherein said methylation-sensitive restriction endonuclease is HhaI.

9. The method of claim 1, wherein said methylation dependent restriction endonuclease digests only methylated DNA.

10. The method of claim 9, wherein said methylation dependent restriction endonuclease is McrBC, McrA, or MrrA.

11. The method of claim 1, wherein said likelihood is determined by matching the methylation ratio of step (d) with reference ratio(s) of the same loci amplified from known tissues/cell types.

12. The method of claim 1, wherein said tissue and/or cell type is blood, saliva, semen, or epidermis.

13. The method of claim 1, wherein the restriction loci are chosen such that they produce distinct methylation ratios for specific tissues and/or cell types.

14. The method of claim 1, wherein said DNA sample is mammalian DNA.

15. The method of claim 14, wherein said mammalian DNA is DNA from a mammal selected from the group consisting of human, ape, monkey, rat, mouse, rabbit, cow, pig, sheep, and horse

16. The method of claim 14, wherein said mammalian DNA is human DNA.

17. The method of claim 16, wherein the human DNA is from a male.

18. The method of claim 16, wherein the human DNA is from a female.

19. The method of claim 1, wherein said amplifying is performed using fluorescently labeled primers.

20. The method of claim 1, wherein signal intensity is determined by separating said amplification products by capillary electrophoresis and then quantifying fluorescence signals.

21. The method of claim 1, wherein amplification and determination of signal intensity are performed by real-time PCR.

22. The method of claim 1, wherein said source is a specific physiological/pathological condition.

23. The method of claim 1, wherein said source is a specific age, or range of ages.

24. The method of claim 1, wherein said source is male.

25. The method of claim 1, wherein said source is female.

26. A method for distinguishing between DNA samples obtained from blood, saliva, semen, and skin epidermis, comprising:
(a) digesting the DNA sample with HhaI;
(b) amplifying the digested DNA with forward and reverse primers for six loci set forth in SEQ ID NOs: 26-31, thereby generating an amplification product for each restriction locus;
(c) determining the intensity of the signal of each amplification product;
(d) calculating methylation ratios for all loci pair combinations;
(e) comparing the methylation ratios calculated in step (d) to a set of reference methylation ratios obtained from DNA from blood, saliva, semen, and skin epidermis; and
(f) calculating the likelihood of each of blood, saliva, semen, and skin epidermis being the source of the DNA, wherein the tissue/cell type with the largest likelihood is determined to be the source of the DNA sample.

27. The method of claim 26, wherein the reference methylation ratio for locus pair SEQ ID NO: 29/SEQ ID NO: 30 in blood is about 0.29.

28. The method of claim 26, wherein the reference methylation ratio for locus pair SEQ ID NO: 29/SEQ ID NO: 30 in semen is about 2.8.

29. The method of claim 26, wherein the reference methylation ratio for locus pair SEQ ID NO: 29/SEQ ID NO: 30 in epidermis is about 0.78.

30. A kit for determining the source of a DNA sample, wherein said kit comprises (a) a single test tube for DNA digestion and amplification using primers for specific genomic loci;
and (b) instructions for calculating at least one methylation ratio and comparing it to reference methylation ratios.

31. The kit of claim 30, wherein the primers comprise forward and reverse primers for the genetic loci set forth in SEQ ID NOs: 26-31.

32. A method for determining whether a DNA sample is from blood, comprising (a) digesting the DNA sample with a methylation-sensitive and/or methylation-dependent restriction endonuclease;
(b) amplifying the digested DNA with at least a first and a second restriction locus, thereby generating an amplification product for each restriction locus;
(c) determining the intensity of the signal of each amplification product;
(d) calculating at least one methylation ratio between the intensity of the signals corresponding to the two restriction loci;
(e) comparing the methylation ratio calculated in step (d) to a set of reference methylation ratios obtained from DNA of known tissues and/or cell types; and
determining whether the DNA sample derives from blood based on likelihood score of blood compared with other tissue and/or cell type likelihood scores.

33. A method for determining whether a DNA sample derives from semen, comprising
(a) digesting the DNA sample with a methylation-sensitive and/or methylation-dependent restriction endonuclease;
(b) amplifying the digested DNA with at least a first and a second restriction locus, thereby generating an amplification product for each restriction locus;
(c) determining the intensity of the signal of each amplification product;
(d) calculating at least one methylation ratio between the intensity of the signals corresponding to the two restriction loci;
(e) comparing the methylation ratio calculated in step (d) to a set of reference methylation ratios obtained from DNA of known tissues and/or cell types; and
(f) determining whether the DNA sample derives from semen based on likelihood score of semen compared with other tissue and/or cell type likelihood scores.

34. A method for determining whether a DNA sample derives from skin epidermis, comprising
(a) digesting the DNA sample with a methylation-sensitive and/or methylation-dependent restriction endonuclease;
(b) amplifying the digested DNA with at least a first and a second restriction locus, thereby generating an amplification product for each restriction locus;
(c) determining the intensity of the signal of each amplification product;
(d) calculating at least one methylation ratio between the intensity of the signals corresponding to the two restriction loci;
(e) comparing the methylation ratio calculated in step (d) to a set of reference methylation ratios obtained from DNA of known tissues and/or cell types; and
(f) determining whether the DNA sample derives from skin epidermis based on likelihood score of skin epidermis compared with other tissue and/or cell type likelihood scores.

35. A method for determining whether a DNA sample derives from saliva, comprising
(a) digesting the DNA sample with a methylation-sensitive and/or methylation-dependent restriction endonuclease;
(b) amplifying the digested DNA with at least a first and a second restriction locus, thereby generating an amplification product for each restriction locus;
(c) determining the intensity of the signal of each amplification product;
(d) calculating at least one methylation ratio between the intensity of the signals corresponding to the two restriction loci;
(e) comparing the methylation ratio calculated in step (d) to a set of reference methylation ratios obtained from DNA of known tissues and/or cell types; and
(f) determining whether the DNA sample derives from saliva based on likelihood score of saliva compared with other tissue and/or cell type likelihood scores.

36. A method for determining whether a DNA sample derives from urine, comprising
(a) digesting the DNA sample with a methylation-sensitive and/or methylation-dependent restriction endonuclease;
(b) amplifying the digested DNA with at least a first and a second restriction locus, thereby generating an amplification product for each restriction locus;
(c) determining the intensity of the signal of each amplification product;
(d) calculating at least one methylation ratio between the intensity of the signals corresponding to the two restriction loci;
(e) comparing the methylation ratio calculated in step (d) to a set of reference methylation ratios obtained from DNA of known tissues and/or cell types; and
determining whether the DNA sample derives from urine based on likelihood score of saliva compared with other tissue and/or cell type likelihood scores.

37. A method for determining whether a DNA sample derives from menstrual blood, comprising
(a) digesting the DNA sample with a methylation-sensitive and/or methylation-dependent restriction endonuclease;
(b) amplifying the digested DNA with at least a first and a second restriction locus, thereby generating an amplification product for each restriction locus;
(c) determining the intensity of the signal of each amplification product;
(d) calculating at least one methylation ratio between the intensity of the signals corresponding to the two restriction loci;
(e) comparing the methylation ratio calculated in step (d) to a set of reference methylation ratios obtained from DNA of known tissues and/or cell types; and
(f) determining whether the DNA sample derives from menstrual blood based on likelihood score of saliva compared with other tissue and/or cell type likelihood scores.

38. A method for determining whether a DNA sample derives from vaginal tissue, comprising
(a) digesting the DNA sample with a methylation-sensitive and/or methylation-dependent restriction endonuclease;
(b) amplifying the digested DNA with at least a first and a second restriction locus, thereby generating an amplification product for each restriction locus;
(c) determining the intensity of the signal of each amplification product;
(d) calculating at least one methylation ratio between the intensity of the signals corresponding to the two restriction loci;
(e) comparing the methylation ratio calculated in step (d) to a set of reference methylation ratios obtained from DNA of known tissues and/or cell types; and
(f) determining whether the DNA sample derives from vaginal tissue based on likelihood score of saliva compared with other tissue and/or cell type likelihood scores.

39. A method for identifying the composition of multiple sources of a DNA sample, comprising:
(a) digesting the DNA sample with a methylation-sensitive and/or methylation-dependent restriction endonuclease;
(b) amplifying the digested DNA with at least a first and a second restriction locus, thereby generating an amplification product for each restriction locus;
(c) determining the intensity of the signal of each amplification product;
(d) calculating at least one methylation ratio between the intensity of the signals corresponding to the two restriction loci;
(e) comparing the methylation ratio calculated in step (d) to a set of reference methylation ratios obtained from DNA of known tissues and/or cell types;
(f) determining the likelihood of each tissue and/or cell type contributing to the source of DNA; and
(g) determining the composition of the source DNA based on the likelihoods obtained in step (f).

40. The method of claim 39, wherein said DNA sample comprises a mixture of DNA from more than one of blood, semen, saliva, skin epidermis, urine, menstrual blood, vaginal tissue.

41. A method for creating a methylation profile of a cell sample, comprising (a) isolating DNA from a cell sample and digesting it with a methylation-sensitive and/or methylation-dependent restriction endonuclease; (b) amplifying the digested DNA with at least a first and a second restriction locus, thereby generating an amplification product for each restriction locus; (c) determining the intensity of the signal of each amplification product; (d) calculating at least one methylation ratio between the intensity of the signals corresponding to the two restriction loci; wherein the calculated methylation ratio(s) comprise the methylation profile of the cell sample.

42. The method of claim 40, comprising comparing the methylation profile of the cell sample with the known methylation profile of at least one cellular reference and determining whether the similarities or differences in the profiles indicates the identity or contamination status of the cell sample.
